(19) United States
(12) Patent Application Publication
(10) Pub. No.: US 2020/0377909 A1
(43) Pub. Date: Dec. 3, 2020

(54) DIRECTED GENOME ENGINEERING USING ENHANCED TARGETED EDITING TECHNOLOGIES

(71) Applicant: MONSANTO TECHNOLOGY LLC, St. Louis, MO (US)

(72) Inventors: Edward James CARGILL, Chesterfield, MO (US); Andrei Y. KOURANOV, St. Louis, MO (US); Linda Ann RYMARQUIS, High Ridge, MO (US)

(73) Assignee: MONSANTO TECHNOLOGY LLC, St. Louis, MO (US)

(21) Appl. No.: 16/886,671

(22) Filed: May 28, 2020

Related U.S. Application Data
(60) Provisional application No. 62/854,146, filed on May 29, 2019.

(51) Int. Cl. C12N 15/90; C12N 9/22; C12N 15/113

(52) U.S. Cl. CPC C12N 15/902; C12N 9/22; C12N 2310/531; C12N 2310/20; C12N 2800/80; C12N 15/113

(57) ABSTRACT

The present disclosure provides methods and compositions for enhancing targeted genome editing and engineering.

### CROSS-REFERENCE TO RELATED APPLICATION

This application claims the benefit of U.S. Provisional Application No. 62/854,146, filed May 29, 2019, which is incorporated by reference in its entirety herein.

### INCORPORATION OF SEQUENCE LISTING

A substitute sequence listing contained in the file named “P34496US01_Corrected_SL.txt” which is 161,845 bytes (measured in MS-Windows®) and created on Aug. 7, 2020, is filed electronically herewith and incorporated by reference in its entirety.

### BACKGROUND

Classic plant or animal breeding relies on chromosomal recombination to develop or introduce desirable traits. The position of such recombination, however, remains largely unpredictable and uncontrollable. Desired chromosomal recombination events also take place at a rather low frequency. The unpredictability and low frequency poses challenges targeted genome engineering, especially at the whole-genome or chromosomal level (e.g., exchange of chromosome arms and translocation of genomic segments). There is a need to develop new technologies to facilitate and improve the efficiency of targeted genome engineering. The instant application provides various approaches (including both compositions and methods) that meet this need.

### SUMMARY

In one aspect, this application provides a genome editing system comprising: a) a nuclease or a first nucleic acid encoding the nuclease; b) a DNA-targeting guide molecule or a second nucleic acid encoding the DNA-targeting guide molecule, wherein the DNA-targeting guide molecule and the nuclease form a multi-unit or single-molecule genome editing system; and c) a tether molecule capable of tethering two entities of the genome editing system, or a third nucleic acid encoding the tether molecule, wherein the tether molecule is an oligonucleotide-based molecule or a cross-linker heterologous to the nuclease.

In another aspect, this application provides a genome editing system comprising: a) two or more site-specific nucleases or a first nucleic acid encoding the two or more site-specific nucleases; and b) a tether molecule or a second nucleic acid encoding the tether molecule, wherein the tether molecule is capable of tethering the two or more site-specific nucleases bound to their corresponding target sites, and wherein the tether molecule is an oligonucleotide-based molecule or a cross-linker heterologous to the nuclease.

In one aspect, this application provides a first genome editing system comprising: a) a CRISPR associated (Cas) nuclease or a nucleic acid encoding the Cas nuclease, wherein the Cas nuclease is coupled to a cross-linker capable of linking two molecules of the Cas nuclease; and b) a first and a second guide RNAs (gRNAs) or one or more nucleic acids encoding the first and second gRNAs, wherein a target sequence of the first gRNA and a target sequence of the second gRNA flank a target genomic segment, and wherein each of the first and second gRNAs is capable of forming a complex with the Cas nuclease.

In one aspect, this application provides a second genome editing system comprising: a) a CRISPR associated (Cas) nuclease or a nucleic acid encoding the Cas nuclease, wherein the Cas nuclease is coupled to a cross-linker capable of linking two molecules of the Cas nuclease; b) a first and a second guide RNAs (gRNAs) or one or more nucleic acids encoding the first and second gRNAs, wherein a target sequence of the first gRNA and a target sequence of the second gRNA flank a target genomic segment, and wherein each of the first and second gRNAs is capable of forming a complex with the Cas nuclease; and c) a first tether guide oligo (tgOligo) corresponding to the first gRNA and a second tgOligo corresponding to the second gRNA, where the first and second tgOligos are capable of hybridizing with each other.

In one aspect, this application provides a third genome editing system comprising: a) a CRISPR associated (Cas) nuclease or a nucleic acid encoding the Cas nuclease, wherein the Cas nuclease is coupled to a cross-linker capable of linking two molecules of the Cas nuclease; b) a first and a second guide RNAs (gRNAs) or one or more nucleic acids encoding the first and second gRNAs, wherein a target sequence of the first gRNA and a target sequence of the second gRNA flank a target genomic segment, and wherein each of the first and second gRNAs is capable of forming a complex with the Cas nuclease; c) a template molecule flanked by a third and a fourth gRNA target sequences; and d) a first tgOligo corresponding to the first gRNA, a second tgOligo corresponding to the second gRNA, a third tgOligo corresponding to the third gRNA, and a fourth tgOligo corresponding to the fourth gRNA, wherein the first and third tgOligos are capable of hybridizing with each other, and wherein the second and fourth tgOligos are capable of hybridizing with each other.

In one aspect, this application provides a fourth genome editing system comprising: a) a CRISPR associated (Cas) nuclease or a nucleic acid encoding the Cas nuclease, wherein the Cas nuclease is coupled to a cross-linker capable of linking two molecules of the Cas nuclease; b) a first and a second guide RNAs (gRNAs) or one or more nucleic acids encoding the first and second gRNAs, wherein a target sequence of the first gRNA and a target sequence of the second gRNA flank a target genomic segment, and wherein each of the first and second gRNAs is capable of forming a complex with the Cas nuclease; c) a deactivated Cas (dCas) nuclease coupled to a cross-linker, or a nucleic acid encoding the dCas nuclease and cross-linker; and d) a third and a fourth gRNAs or one or more nucleic acids encoding the third and fourth gRNAs, wherein target sequences of the third and fourth gRNAs are within and on the opposite ends of the target genomic segment, and wherein a dCas nuclease bound to the third or fourth gRNA target sequence is capable of dimerizing with a Cas nuclease bound to a gRNA target sequence on the opposite end of the target genomic segment.

In one aspect, this application provides a fifth genome editing system comprising: a) a CRISPR associated (Cas) nuclease or a nucleic acid encoding the Cas nuclease, wherein the Cas nuclease is coupled to a cross-linker capable of linking two molecules of the Cas nuclease; b) a first and a second guide RNAs (gRNAs) or one or more nucleic acids encoding the first and second gRNAs, wherein a target sequence of the first gRNA and a target sequence of the second gRNA flank a target genomic segment, and wherein each of the first and second gRNAs is capable of forming a complex with the Cas nuclease; and c) a template molecule flanked by two gRNA target sequences, wherein each end of the template molecule comprises a sequence homologous to a sequence flanking the target genomic segment.

In one aspect, this application provides a sixth genome editing system comprising: a) a CRISPR associated (Cas) nuclease or a nucleic acid encoding the Cas nuclease, wherein the Cas nuclease is coupled to a cross-linker capable of linking two molecules of the Cas nuclease; b) a first and a second guide RNAs (gRNAs) or one or more nucleic acids encoding the first and second gRNAs, wherein a target sequence of the first gRNA and a target sequence of the second gRNA flank a target genomic segment, and wherein each of the first and second gRNAs is capable of forming a complex with the Cas nuclease; and c) a template molecule flanked by two gRNA target sequences, wherein each end of the template molecule comprises a sequence homologous to a sequence flanking the target genomic segment; and d) a deactivated Cas (dCas) nuclease or a nucleic acid encoding the dCas nuclease, wherein the dCas nuclease is coupled to a cross-linker and capable of being bound to the two gRNA target sequences on the template molecule.

In one aspect, this application provides a seventh genome editing system comprising: a) a Cas nuclease or a nucleic acid encoding the Cas nuclease; b) a first and a second gRNAs or one or more nucleic acids encoding the first and second gRNAs, wherein a target sequence of the first gRNA and a target sequence of the second gRNA flank a target genomic segment; and c) a first tgOligo corresponding to the first gRNA and a second tgOligo corresponding to second gRNA, wherein the first and second tgOligos are capable of hybridizing with each other.

In one aspect, this application provides an eighth genome editing system comprising: a) a Cas nuclease or a nucleic acid encoding the Cas nuclease, wherein the Cas nuclease is coupled to a cross-linker; b) a first and a second gRNAs or one or more nucleic acids encoding the first and second gRNAs, wherein a target sequence of the first gRNA and a target sequence of the second gRNA flank a target genomic segment; c) a first tgOligo corresponding to the first gRNA and a second tgOligo corresponding to second gRNA, wherein the first and second tgOligos are capable of hybridizing with each other; d) a deactivated Cas (dCas) nuclease coupled to a cross-linker, or a nucleic acid encoding the dCas nuclease and cross-linker; and e) a third and a fourth gRNAs or one or more nucleic acids encoding the third and fourth gRNAs, wherein target sequences of the third and fourth gRNAs are within and on the opposite ends of the target genomic segment; and wherein a dCas nuclease bound to the third or fourth gRNA target sequence is capable of dimerizing with a Cas nuclease bound to a gRNA target sequence on the opposite end of the target genomic segment.

In one aspect, this application provides a ninth genome editing system comprising: a) a Cas nuclease or a nucleic acid encoding the Cas nuclease; b) a first and a second gRNAs or one or more nucleic acids encoding the first and second gRNAs, wherein a target sequence of the first gRNA and a target sequence of the second gRNA flank a target genomic segment; and c) a first tgOligo corresponding to the first gRNA and a second tgOligo corresponding to second gRNA, wherein the first and second tgOligos are capable of hybridizing with each other, wherein the first and second tgOligos are capable of hybridizing and forming a double-stranded template sequence for integration.

In one aspect, this application provides a tenth genome editing system comprising: a) a Cas nuclease or a nucleic acid encoding the Cas nuclease; b) a first and a second gRNAs or one or more nucleic acids encoding the first and second gRNAs, wherein a target sequence of the first gRNA and a target sequence of the second gRNA flank a target genomic segment, c) a first tgOligo corresponding to the first gRNA and further capable of hybridizing with the target genomic segment on the opposite end of the first gRNA target site, and d) a second tgOligo corresponding to the second gRNA and further capable of hybridizing with the target genomic segment on the opposite end of the second gRNA target site.

In one aspect, this application provides a eleventh genome editing system comprising: a) a Cas nuclease or a nucleic acid encoding the Cas nuclease; b) a first and a second gRNAs or one or more nucleic acids encoding the first and second gRNAs, wherein a target sequence of the first gRNA and a target sequence of the second gRNA flank a target genomic segment; c) a first tgOligo corresponding to the first gRNA and a second tgOligo corresponding to the second gRNA; d) one or more double-strand oligos (dsOligos) with two overhangs, wherein each of the two overhangs is capable of hybridizing with the first or second tgOligos.

In one aspect, this application provides a first method for chromosome engineering comprising: introducing into a target cell a genome editing system described herein, and producing a modified chromosome comprising a deletion or inversion of the target genomic segment or a replacement of the target genomic segment based on the template molecule.

In one aspect, this application provides a second method for chromosome engineering comprising: introducing into a target cell a genome editing system comprising: a) a Cas nuclease coupled to a cross-linker or one or more nucleic acids encoding the Cas nuclease and cross-linker, wherein the cross-linker is capable of linking two Cas nuclease molecules; and b) a first and a second gRNAs or one or more nucleic acids encoding the first and second gRNAs, and wherein the first and second gRNAs have target sequences in a first recombination region of interest on a pair of donor and recipient chromosomes; and producing a recombinant chromosome comprising a portion of the donor chromosome and a portion of the recipient chromosome.

In one aspect, this application provides a third method for chromosome engineering comprising: introducing into a target cell a genome editing system comprising: a) a Cas nuclease coupled to a cross-linker or a nucleic acid encoding the Cas nuclease and cross-linker, wherein the cross-linker is capable of linking two molecules of the Cas nuclease; b) a first and a second gRNAs or one or more nucleic acids encoding the first and second gRNAs, and wherein the first and second gRNAs have target sequences in a first recombination region of interest on a pair of donor and recipient chromosomes; and c) a third and a fourth gRNAs or one or more nucleic acids encoding the third and fourth gRNAs, and wherein the third and fourth gRNAs have target sequences in a second recombination region of interest on the pair of donor and recipient chromosomes; and producing a recombinant chromosome comprising a portion of the donor chromosome and a portion of the recipient chromosome, wherein the method is capable of producing a recombinant chromosome comprising a backbone from the recipient chromosome with a chromosome segment integrated from the donor chromosome between the first and second recombination regions of interest.

In one aspect, this application provides a fourth method for chromosome engineering comprising: introducing into a target cell a genome editing system comprising: a) a Cas nuclease coupled to a cross-linker or a nucleic acid encoding the Cas nuclease and cross-linker, wherein the cross-linker is capable of linking two molecules of the Cas nuclease; b) a first and a second gRNAs or one or more nucleic acids encoding the first and second gRNAs, and wherein the first and second gRNAs have target sequences in a first recombination region of interest on a pair of donor and recipient chromosomes; and c) a first tgOligo corresponding to the first gRNA, a second tgOligo corresponding to the second gRNA, and wherein the first and second tgOligos are capable of hybridizing with each other; and producing a recombinant chromosome comprising a portion of the donor chromosome and a portion of the recipient chromosome.

In one aspect, this application provides a fifth method for chromosome engineering comprising: introducing into a target cell a genome editing system comprising: a) a Cas nuclease or a nucleic acid encoding the Cas nuclease; b) a first and a second gRNAs or one or more nucleic acids encoding the first and second gRNAs, and wherein the first and second gRNAs have target sequences in a first recombination region of interest on a pair of donor and recipient chromosomes; and c) a first tgOligo corresponding to the first gRNA, a second tgOligo corresponding to the second gRNA, and wherein the first and second tgOligos are part of a single molecule or are capable of hybridizing with each other; and producing a recombinant chromosome comprising a portion of the donor chromosome and a portion of the recipient chromosome.

In one aspect, this application provides a sixth method for chromosome engineering comprising: introducing into a target cell a genome editing system comprising: a) a Cas nuclease coupled to a single-strand nucleic acid-binding domain heterologous to the Cas nuclease or a nucleic acid encoding the Cas nuclease and the single-strand nucleic acid-binding domain, b) a first and a second gRNAs or one or more nucleic acids encoding the first and second gRNAs, wherein the first and second gRNAs have target sequences in a first recombination region of interest on a pair of donor and recipient chromosomes, c) a first tgOligo corresponding to the first gRNA and a second tgOligo corresponding to the second gRNA, wherein the first, second, or both tgOligos comprise a hairpin configuration until a portion of the tgOligo sequence hybridizes with an intended genomic sequence, and wherein the non-hybridized portion of the first, second, or both tgOligos unfolds into a single-strand form upon the hybridization and further binds the single-strand nucleic acid-binding domain; producing a recombinant chromosome comprising a portion of the donor chromosome and a portion of the recipient chromosome.

In one aspect, this application further provides a twelfth genome editing system comprising: a) a CRISPR associated (Cas) nuclease or a nucleic acid encoding the Cas nuclease; and b) a first and a second guide RNAs (gRNAs) or one or more nucleic acids encoding the first and second gRNAs, wherein the first and second gRNAs have target sequences arranged such that the double-strand DNA cleavage mediated by the first and second gRNAs is capable of creating two 3′ free ends from non-target strands complementing each other.

In one aspect, this application further provides a method for chromosome engineering comprising: introducing into a target cell a genome editing system comprising: a) a first and a second CRISPR associated (Cas) nucleases or one or more nucleic acids encoding the first and second Cas nucleases, and b) a first and a second guide RNAs (gRNAs) or one or more nucleic acids encoding the first and second gRNAs, wherein the first and second gRNAs are capable of binding with the first and second Cas nucleases, which mediate double-strand DNA cleavage, wherein the first and second gRNAs have target sequences arranged such that the double-strand DNA cleavage is capable of creating two 3′ free ends from non-target strands complementing each other, and wherein the first and second gRNA target sequences are in a recombination region of interest on a pair of donor and recipient chromosomes; and producing a recombinant chromosome comprising a portion of the donor chromosome and a portion of the recipient chromosome.

In one aspect, this application provides a thirteenth genome editing system comprising: a) a CRISPR associated (Cas) nuclease or a nucleic acid encoding the Cas nuclease, b) a first and a second guide RNAs (gRNAs) or one or more nucleic acids encoding the first and second gRNAs, c) a chimeric tgOligo comprising sequences capable of recognizing the target sites of both the first and second gRNAs and binding both non-target strand 3′ free ends generated from DNA cleavage mediated by the Cas nuclease.

In one aspect, this application further provides a method for chromosome engineering comprising: introducing into a target cell a thirteenth genome editing system described above, wherein a first and a second gRNA target sequences are in a recombination region of interest on a pair of donor and recipient chromosomes, and producing a recombinant chromosome comprising a portion of the donor chromosome and a portion of the recipient chromosome.

In one aspect, this application further provides a method for chromosome engineering comprising introducing into a target cell a genome editing system comprising: (a) a Cas nuclease or a nucleic acid encoding the Cas nuclease; (b) a first and a second gRNAs or one or more nucleic acids encoding the first and second gRNAs, and where the first and second gRNAs have target sequences in a first recombination region of interest on a pair of donor and recipient chromosomes; and (c) a first tgOligo corresponding to the first gRNA, a second tgOligo corresponding to the second gRNA, and where the first and second tgOligos are part of a single molecule or are capable of hybridizing with each other; producing a recombinant chromosome comprising a portion of said donor chromosome and a portion of the recipient chromosome.

In one aspect, this application further provides a method for chromosome engineering comprising introducing into a target cell a genome editing system comprising: (a) a Cas nuclease coupled to a single-strand nucleic acid-binding domain heterologous to the Cas nuclease or a nucleic acid encoding the Cas nuclease and said single-strand nucleic acid-binding domain, (b) a first and a second gRNAs or one or more nucleic acids encoding the first and second gRNAs, where the first and second gRNAs have target sequences in a first recombination region of interest on a pair of donor and recipient chromosomes, (c) a first tgOligo corresponding to the first gRNA and a second tgOligo corresponding to the second gRNA, where the first, second, or both tgOligos comprise a hairpin configuration until a portion of the tgOligo sequence hybridizes with an intended genomic sequence, and where the non-hybridized portion of the first, second, or both tgOligos unfolds into a single-strand form upon the hybridization and further binds the single-strand nucleic acid-binding domain; producing a recombinant chromosome comprising a portion of the donor chromosome and a portion of said recipient chromosome.

In one aspect, this disclosure further provides a genome editing system comprising: (a) a CRISPR associated (Cas) nuclease or a nucleic acid encoding the Cas nuclease; and (b) a first and a second guide RNAs (gRNAs) or one or more nucleic acids encoding the first and second gRNAs, where the first and second gRNAs have target sequences arranged such that the double-strand DNA cleavage mediated by the first and second gRNAs is capable of creating two 3′ free ends from non-target strands complementing each other.

### BRIEF DESCRIPTION OF THE DRAWINGS

FIG. 1: Schematic of a Cas9-mediated double-stranded break (DSB) and a tether guide oligo (tgOligo) bound to a target DNA site. The Cas9-PAM interaction occurs on the non-target strand; sgRNA-DNA annealing occurs on the target strand. The blunt ends at the Cas9 cut site are held in place by Cas9 at the 5′ end of the non-target strand (PAM location), and at both cut ends (3′ and 5′) of the target strand. The 3′ cut end of the non-target strand is free and ‘flaps’ around. The 3′ free ‘flap’ end of the non-target strand can be up to 35 nucleotides which can be sufficient for specific complementarity binding. A tgOligo (e.g., a ssDNA template) can be included for integration of desired nucleotide modification. The drawing scheme used here is followed in the subsequent figures.

FIG. 2: Illustration of Cas9 conjugated with a homodimer domain (top left), heterodimer domains (second row from top), and a ssDNA binding domain (top right) to facilitate dimerization. Ligands for the homodimer and heterodimer domains are shown. ssDNA is shown as a squiggle. A single ssDNA molecule may facilitate dimerization by binding to multiple Cas9-ssDNA binding domain fusion proteins via the ssDNA binding domains in those fusion proteins. Alternatively, two or more single ssDNA molecules may be partially complementary to form duplex regions so that the duplex regions facilitate dimerization of two Cas9-ssDNA binding domain fusion proteins which each bind to single-stranded sections of ssDNA molecules. The drawing scheme used here is followed in the subsequent figures; e.g., the ligands, the homodimer or heterodimer domains, ssDNA binding domains. Each component of the Cas9/sgRNA complex and target DNA are shown as illustrated in FIG. 1. The drawing scheme used here for different dimerization or ssDNA binding domains is followed in the subsequent figures.

FIG. 3: Use of catalytically deactivated Cas9 (dCas9) to increase genome editing efficiency. Panel 1 illustrates that dCas9 binds to DNA at a target site specified by the gRNA and creates a loop structure accessible for template-based editing. Panel 2 illustrates a modified scheme for further facilitating template-based editing via a dCas9 conjugated with a ssDNA-binding domain. The editing efficiency with this modified scheme is expected to be higher compared to those in Panel 1, because a ssDNA template is bound to dCas9 complex and would be brought into proximity of the gRNA target.

FIG. 4: An example construct containing Cas9, gRNAs, and tgOligos. RZ stands for Ribozyme, an enzyme that cleaves a 15 bp recognition site in RNA (RZ site).

FIG. 5: Illustration of a basic two-gRNA approach (e.g., two Cas9/gRNA complexes flanking a target genomic region) for achieving INDELs or complete inversion. Two configurations are shown where the two gRNAs recognize the same DNA strand or the opposite strands. With two Cas9/gRNA complexes, the flanked genomic region is most often deleted and NHEJ repair combining the two cut sites back together. There is also occurrence of INDEL (insertion/deletion) mutations at either Cas9+gRNA flanking site. It is also possible to recover with lower frequency complete inversions of the flanked genomic region.

FIG. 6: Illustration of various approaches for improving genome editing efficiency. Using dimerization domains (See FIG. 2), tgOligos (See FIG. 1), or a combination of both can enhance recovery of complete knockout (deletion) of the genomic region flanked by the two gRNA target sites. Panel 1 shows a dimerization-enhanced knock out (KO) event. Panel 2 shows a tgOligo-enhanced KO event. Panel 3 shows an enhanced KO event via a combination of dimerization and tgOligos. Panel 4 shows a tgOligo-enhanced inversion event. Panel 5 shows a dimerization-enhanced inversion event. Panel 6 shows an inversion event assisted by a combination of Cas9 dimerization/deactivation and tgOligos. Only shown is the configuration where two gRNAs recognize different strands of a target dsDNA. The same concept is equally applicable to the other configuration where two gRNAs recognize the same strand of a target dsDNA.

FIG. 7: Illustration of editing the corn BR2 gene to generate a dominant knockout allele via genome inversion. Two gRNAs are used. A first gRNA (shown on the left) targets the end of the first exon of BR2; a second gRNA (shown on the right) recognizes the start codon region of the adjacent GRMZM2G491632 gene. Inversion of the genomic segment flanked by these two gRNAs can lead to a BR2 antisense partial transcript (See Transcript 1). This BR2 antisense transcript is produced via the GRMZM2G491632 promoter activity. Adjusting the relative position of the two gRNAs can achieve a BR2 antisense complete transcript (e.g., moving the first gRNA on the left to target the start codon region of the BR2 gene) or a BR2 antisense transcript under the control of the native BR2 promoter (e.g., moving the second gRNA on the right to target the stop codon region of the BR2 gene).

FIG. 8: Illustration of dimerization-enhanced template-based editing or site directed integration (SDI) at a single location (Panels 1 and 2) or multiple locations (Panel 3), and dimerization/tgOligo-enhanced template-based editing or SDI (Panel 4).

FIG. 9: Illustration of template editing, site directed integration, and/or recombination with tgOligos.

FIG. 10: Further illustration of using tgOligos to enhance template-based genome editing or site directed integration. For example, two Cas9/gRNA complexes flank a region of interest on opposite target strands. Two tgOligos with complementarity to 3′ free flaps at flanking sites and further including complementary regions between the two tgOligo are used. Here, the tgOligo can serve as a template for editing or provide a desired sequence for site directed integration.

FIG. 11: Further illustration of using tgOligos and coupled with double-strand oligos (dsOligos) to enhance template-based genome editing or site directed integration. Here, dsOligos with complementary overhangs and further complementarity with tgOligos are used to serve as a larger template for site directed integration or editing.

FIG. 12: Illustration of cis or trans chromosome arm exchange using dimerization domains (Panel 1), tgOligos (Panel 2), a dimerization/tgOligo combination at the same site (Panel 3) or at different sites (Panel 4), and with ssDNA binding domains combined with hairpin tgOligos (Panel 5).

FIG. 13: Further illustration of using induced homo or hetero dimerization technology to facilitate targeted chromosome arm exchange in crops. Dimerization can be induced by chemicals, light, or other stimulants.

FIG. 14: Comparison of mutant alleles in maize brachytic 2 (BR2) gene and the use of genome editing-assisted recombination to stack two mutant alleles/polymorphisms. The br2-NA/MX allele carries a 4.7 kb insertion (triangle) in Exon 5. The br2-Italian allele carries a 579 bp insertion Intron 4 (triangle).

FIG. 15: Illustration of a cis genomic fragment exchange using dimerization domains (Panel 1), tgOligos (Panel 2), a dimerization/tgOligo combination at the same site (Panel 3) or at different sites (Panel 4). The same concepts from FIG. 12 and earlier are applied to flank a genomic segment on homologous (cis) chromosomes and exchange the flanked segment. Dimerization domains, tgOligos, or their combination can enhance the efficiency of the exchange.

FIG. 16: Illustration of a trans genomic fragment exchange using dimerization domains (Panel 1), tgOligos (Panel 2), a dimerization/tgOligo combination at the same site (Panel 3) or at different sites (Panel 4). The same concepts from FIG. 15 and earlier are applied to flank a genomic segment on non-homologous (trans) chromosomes and exchange the flanked segment. Dimerization domains, tgOligos, or their combination can enhance the efficiency of the exchange, especially given the regions would not share homology for native DNA repair facilitation.

FIG. 17: Schematic showing TLRs 7 and 8 genes are adjacent on the X chromosome in cattle.

FIG. 18: Illustration of hairpin tgOligos and ssDNA binding domains to facilitate chromosome editing. The tgOligos would be in a hairpin formation unless bound to the 3′ free flap of the nuclease DSB. When bound to the 3′ free flap, the tgOligo would be in a single strand form (squiggle line in FIG. 18) accessible to a single strand binding domain that could be attached to the editing complex (purple (pacman shape) in FIG. 18). This can allow the recognition and binding of only tgOligos bound to the DSB junctions so that they are brought together in proximity to facilitate a recombination event.

FIG. 19: Illustration of a single sgRNA+tgRNA molecule to facilitate inversion of a flanked genomic segment.

FIG. 20: Illustration of the stacking of an inverted Y1 gene head-to-tail to produce an antisense transcript to silence the gene expression. This approach can create a dominant mutant Y1 allele for a normally recessive trait. This dominant allele remains controlled by the native Y1 promoter.

FIG. 21: Illustration of a tgOligo-free approach for linking two Cas-mediated double-strand breaks using complementary non-target strand 3′ free flaps. This approach can be used to guide DNA repair to create chromosome exchanges or deletions. Essentially, two gRNAs are designed to cut two genomic locations such that complementary flaps are created. One option is to use two different Cas9 proteins that have different PAM specificities. Then, gRNAs are chosen to target two sites—each with a different PAM. Differences in the spacer target could also be used to produce two complementary flaps. For example, if two target sequences vary by one or a few nucleotides, two different gRNA can be designed for these two target sites with specificity. The two 3′ free flaps resulted from this design would be complementary to each other even though they may have mismatch at a few base pairs.

FIG. 22: Further illustration of a tgOligo-free approach by generating complementary non-target strand 3′ free using a pair of complementary spacers (also known as gRNA guide sequences). For example, gRNAs are designed to cut two genomic locations such that complementary flaps are created. This can be done by designing gRNAs that compete with each other for a shared genomic site. If sequences at both sites are identical, two possible flaps could be produced at each site. Two out of four configurations produce complementary flaps (Panels 1 and 2). The other two configurations produce identical (not complementary) flaps (Panels 3 and 4). If sequences are not identical between target sites, then spacers can be designed to only bind one of the two sites and then only complementary flaps would be produced.

FIG. 23: Illustration of a chimeric tgOligo with a hairpin configuration. A chimeric tgOligo can recognize target sites of two separate gRNAs and bind two separate 3′ free flaps ends generated from DNA cleavage mediated by the two gRNAs. A chimeric tgOligo linking two gRNA target site can be used to promote chromosome translocation. The illustrated chimeric tgOligo also exhibits a hairpin configuration until a portion of the tgOligo sequence hybridizes with an intended genomic sequence.

### DETAILED DESCRIPTION

This application provides various approaches to modify targeted editing techniques for facilitating, and further increasing efficiency of targeted chromosome engineering.

In one aspect, a disclosed approach is to integrate site-directed nucleases and induced protein dimerization technologies. For example, this application describes modifying a site-directed nuclease with a protein dimerization domain and allowing a modified nuclease to create targeted chromosomal breaks at different locations in a genome. Protein dimerization can be induced by applying chemical, light, or other induction signals. Without being bound to any scientific theory, the induced dimerization results in cross linking between modified nucleases and thereby brings two genomic sites with chromosomal breaks into close vicinity. The direct linking of chromosomal breaks would increase efficiency and frequency of desired cis or trans chromosomal arm exchange, or other type of chromosomal rearrangements.

Various protein dimerization technologies (including induced and non-induced dimerization) can be used here. Many such technologies have been used for protein-protein interaction studies in different systems including plants (Andersen et al., Scientific Reports 6, Article number: 27766 (2016); Miyamoto et al., Nature Chemical Biology 8 (5): 465-70 (2012)). Some are also commercially available. For example, iDimerize is a chemically induced dimerization system from TAKARA/Clontech Laboratories, Inc. In one aspect, this iDimerize technology can be used in targeted chromosome engineering.

In another aspect, a disclosed approach is to design and utilize a tether guide oligo (tgOligo) molecule to bring into close proximity two or more genomic loci with targeted chromosomal breaks created by site-directed nucleases. Similar to the nuclease dimerization-based approach and without being bound to any scientific theory, the cross-linking or tethering (and hence close vicinity) of targeted chromosomal breaks can increase efficiency and frequency of desired cis or trans chromosomal arm exchange, or other type of chromosomal rearrangements. Chromosomal recombination events with desired chromosomal exchange can be identified by molecular methods including, for example, PCR and deep sequencing, or genotyping at a later breeding generation.

In one aspect, this application provides a genome editing system comprising: a) a nuclease or a first nucleic acid encoding the nuclease; b) a DNA-targeting guide molecule or a second nucleic acid encoding the DNA-targeting guide molecule, wherein the DNA-targeting guide molecule and the nuclease form a multi-unit or single-molecule DNA binding machinery; and c) a tether molecule capable of tethering two entities of the DNA binding machinery, or a third nucleic acid encoding the tether molecule, wherein the tether molecule is an oligonucleotide-based molecule or a cross-linker heterologous to the nuclease.

In another aspect, this application provides a genome editing system comprising: a) two or more site-specific nucleases or a first nucleic acid encoding the two or more site-specific nucleases; and b) a tether molecule or a second nucleic acid encoding the tether molecule, wherein the tether molecule is capable of tethering the two or more site-specific nucleases bound to their corresponding target sites, and wherein the tether molecule is an oligonucleotide-based molecule or a cross-linker heterologous to the nuclease.

In one aspect, a genome editing system provided here comprises a functional nuclease. In another aspect, a genome editing system comprises a deactivated nuclease. In one aspect, a nuclease comprises a FokI nuclease domain. In another aspect, a nuclease is a RNA-guided nuclease. In a further aspect, a nuclease is a Clustered Regularly Interspaced Short Palindromic Repeats (CRISPR) associated nuclease (Cas nuclease). In another aspect, a nuclease is selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1 (also known as Cas12a), and a homolog or modified version thereof. In another aspect, a nuclease is a Cas9 nuclease or a homolog or modified version thereof. In one aspect, a nuclease is a Cas9 protein, or a modified version thereof, from Streptococcus pyogenes, Streptococcus thermophilius, Staphylococcus aureus, Neisseria meningitides, or Treponema denticola. In another aspect, a nuclease is Cpf1 or a homolog or modified version thereof.

In one aspect, a genome editing system provided here comprises a RNA molecule as a DNA-targeting guide molecule. In another aspect, a DNA-targeting guide molecule is selected from the group consisting of a CRISPR guide RNA, a TAL effector domain, and a zinc finger domain.

In one aspect, a genome editing system provided here comprises a tgOligo as a tether molecule. In another aspect, a tether molecule is a cross-linker coupled to a nuclease or a DNA-targeting guide molecule. In a further aspect, a tether molecule is a dimerization domain coupled to a nuclease.

In one aspect, a genome editing system provided here comprises a nuclease-coding nucleic acid molecule that is codon optimized for a eukaryotic cell. In another aspect, a nuclease-coding nucleic acid molecule is codon optimized for a plant cell. In another aspect, a nuclease-coding nucleic acid molecule is codon optimized for a monocot species. In a further aspect, a nuclease-coding nucleic acid molecule is codon optimized for a corn or soybean.

In one aspect, a first nucleic acid, a second nucleic acid, a third nucleic acid, or any combination thereof, in a genome editing system provided here is operably linked to a regulatory element operable in a target cell. In another aspect, a combination of two or more of the first nucleic acid, the second nucleic acid, and the third nucleic acid are in a single molecule.

In one aspect, a tether molecule is capable of tethering two or more DNA binding machineries bound to two genomic loci. In another aspect, a tether molecule is capable of tethering two or more DNA binding machineries bound to two genomic loci located in in a single chromosome flanking a target genomic region. In another aspect, a tether molecule is capable of tethering two or more DNA binding machineries bound to two genomic loci are on separate chromosomes.

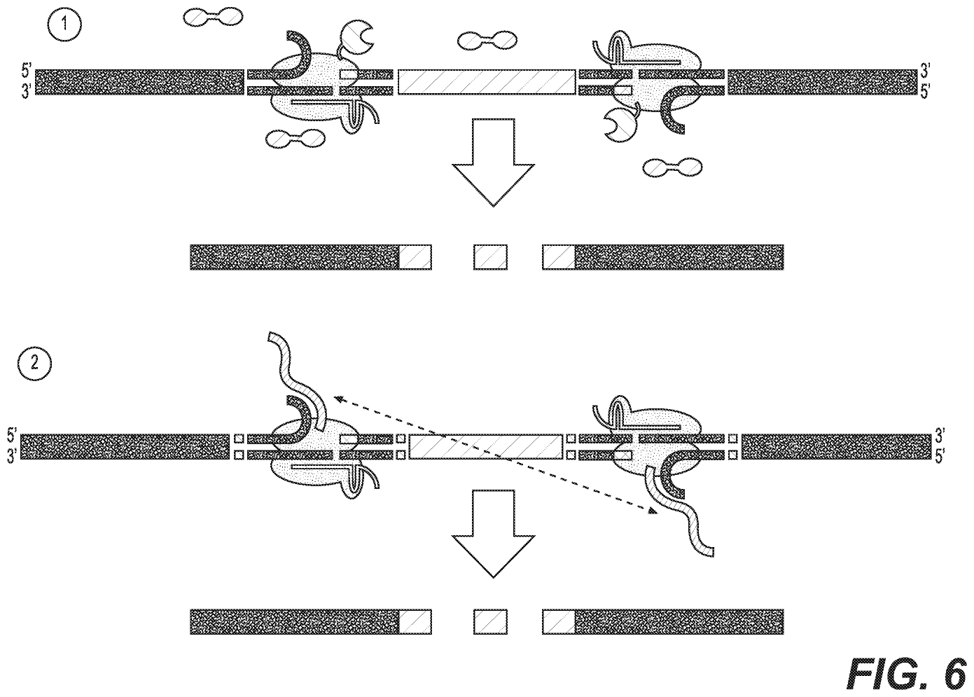

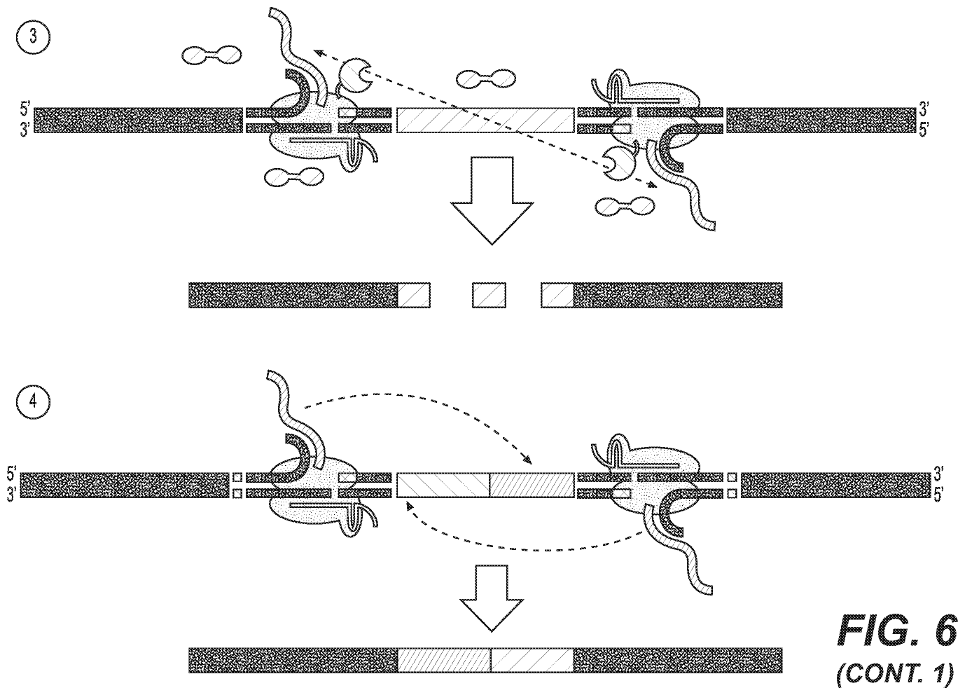

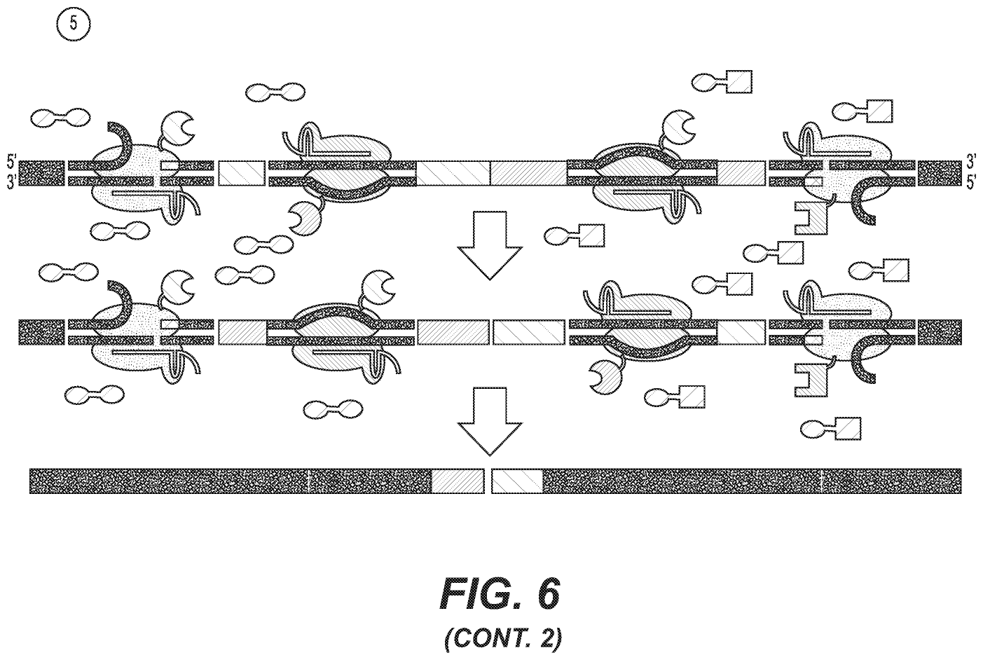

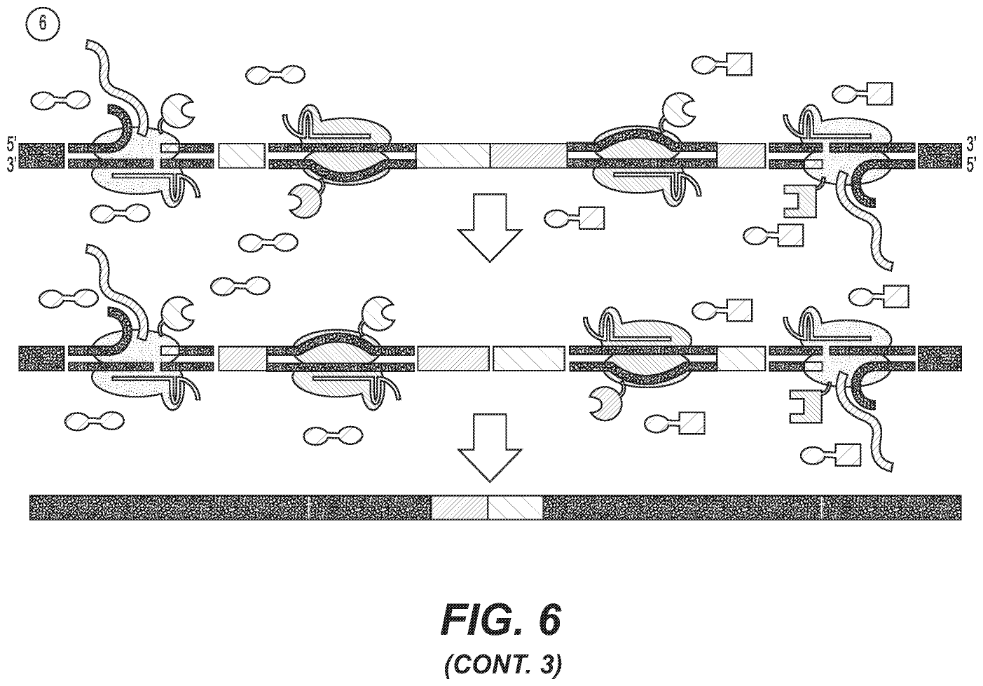

In one aspect, this application provides a first genome editing system comprising: a) a CRISPR associated (Cas) nuclease or a nucleic acid encoding the Cas nuclease, wherein the Cas nuclease is coupled to a cross-linker capable of linking two molecules of the Cas nuclease; and b) a first and a second guide RNAs (gRNAs) or one or more nucleic acids encoding the first and second gRNAs, wherein a target sequence of the first gRNA and a target sequence of the second gRNA flank a target genomic segment, and wherein each of the first and second gRNAs is capable of forming a complex with the Cas nuclease. An exemplary graphic illustration is depicted in FIG. 6, panel 1. In one aspect, target sequences of a first and a second gRNAs are on the opposite strands of a target genomic segment. In another aspect, a cross-linker is a homo-dimerization domain. In another aspect, a cross-linker is a hetero-dimerization domain. In another aspect, a cross-linker requires a cross-linking ligand. In another aspect, a cross-linker is an inducible dimerization domain. In another aspect, a cross-linker is a single-strand DNA or RNA binding domain.

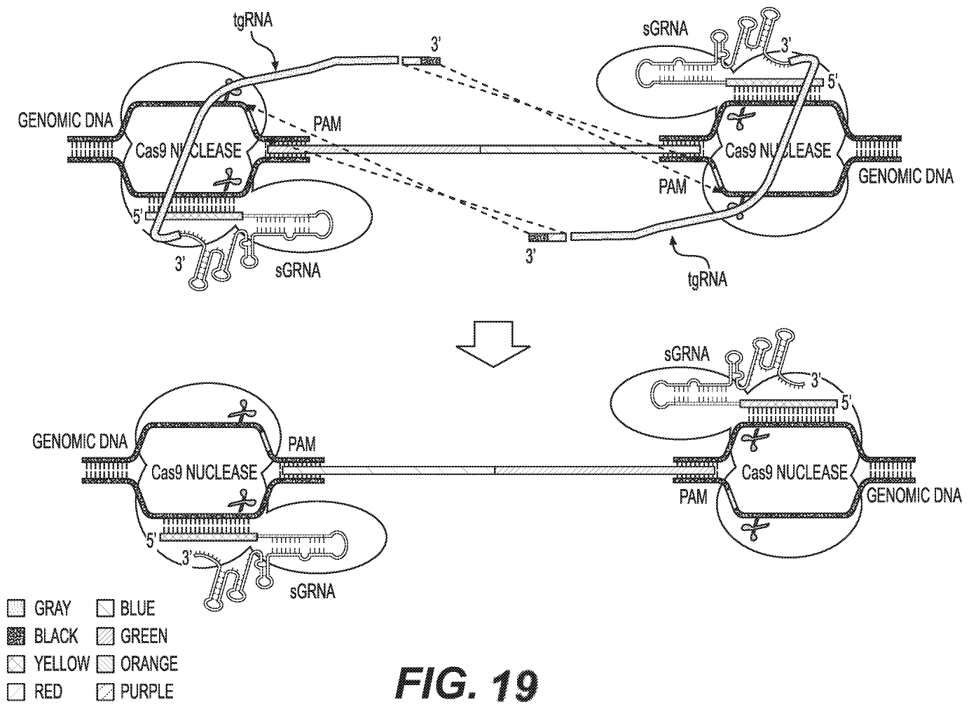

In one aspect, this application provides a second genome editing system comprising: a) a CRISPR associated (Cas) nuclease or a nucleic acid encoding the Cas nuclease, wherein the Cas nuclease is coupled to a cross-linker capable of linking two molecules of the Cas nuclease; b) a first and a second guide RNAs (gRNAs) or one or more nucleic acids encoding the first and second gRNAs, wherein a target sequence of the first gRNA and a target sequence of the second gRNA flank a target genomic segment, and wherein each of the first and second gRNAs is capable of forming a complex with the Cas nuclease; and c) a first tether guide oligo (tgOligo) corresponding to the first gRNA and a second tgOligo corresponding to the second gRNA. In another aspect, a first and a second tgOligos are capable of hybridizing with each other. An exemplary graphic illustration is depicted in FIG. 6, panel 3. In one aspect, a first, a second, or both tgOligos comprise a hairpin configuration until a portion of the tgOligo sequence hybridizes with an intended genomic sequence. In another aspect, the non-hybridized portion of a first, a second, or both tgOligos unfolds into a single-strand form upon the hybridization. In a further aspect, a first and a second tgOligos are in a single molecule. In another aspect, a first and a second gRNAs are part of a first tgRNA and a second tgRNA, respectively, wherein the first tgRNA has a tether site adjacent to the target site of the second gRNA, and wherein the second tgRNA has a tether site adjacent to the target site of the first gRNA. In a further aspect, a first tgRNA tether site comprises, or is immediately adjacent to, the PAM sequence of a second gRNA, and wherein a second tgRNA tether site comprises, or is immediately adjacent to, the PAM sequence of a first gRNA. An exemplary graphic illustration is depicted in FIG. 19.

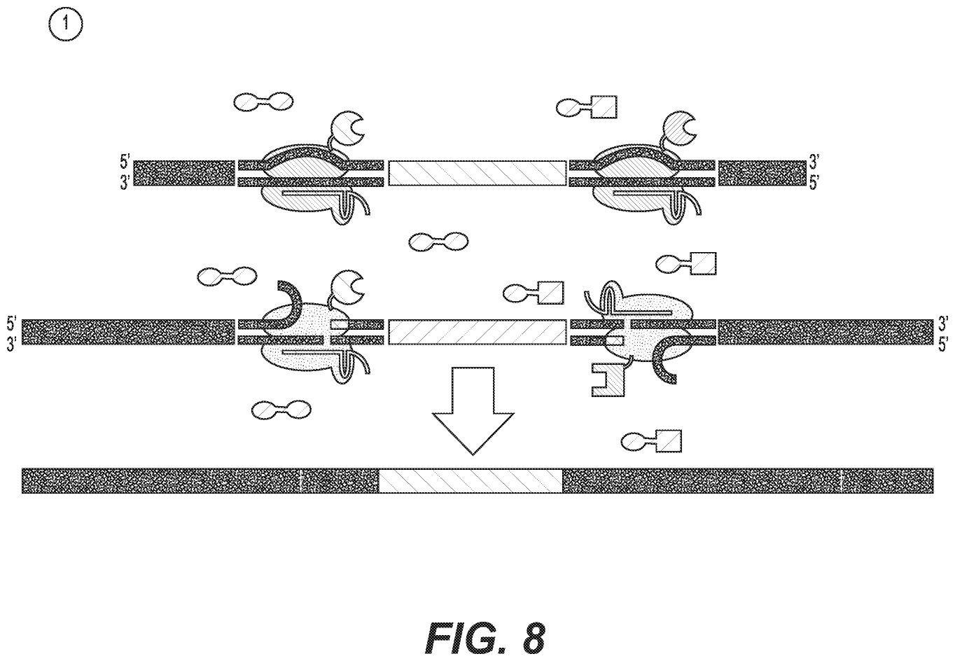

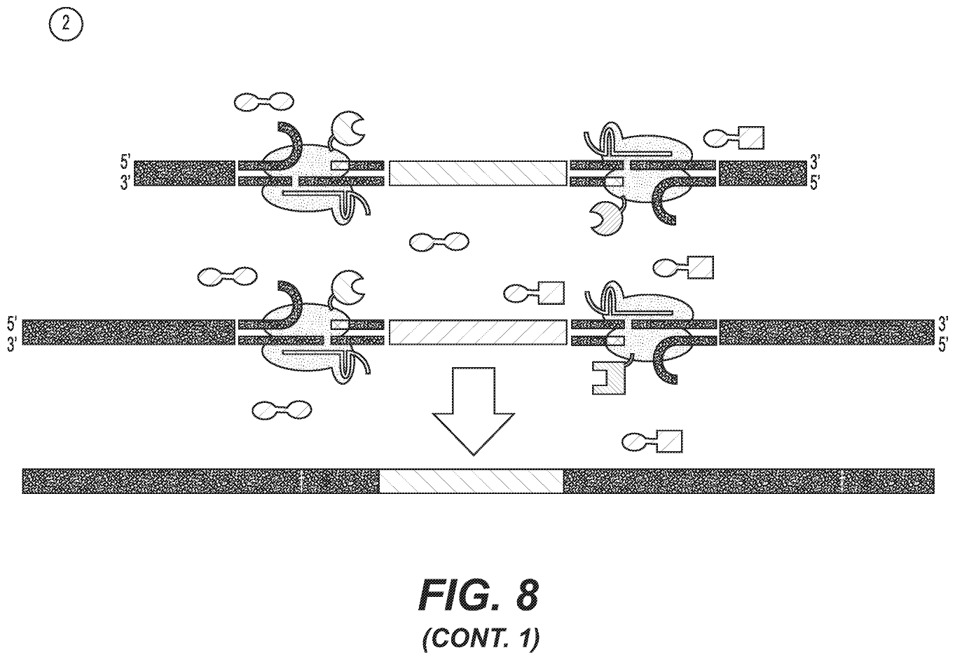

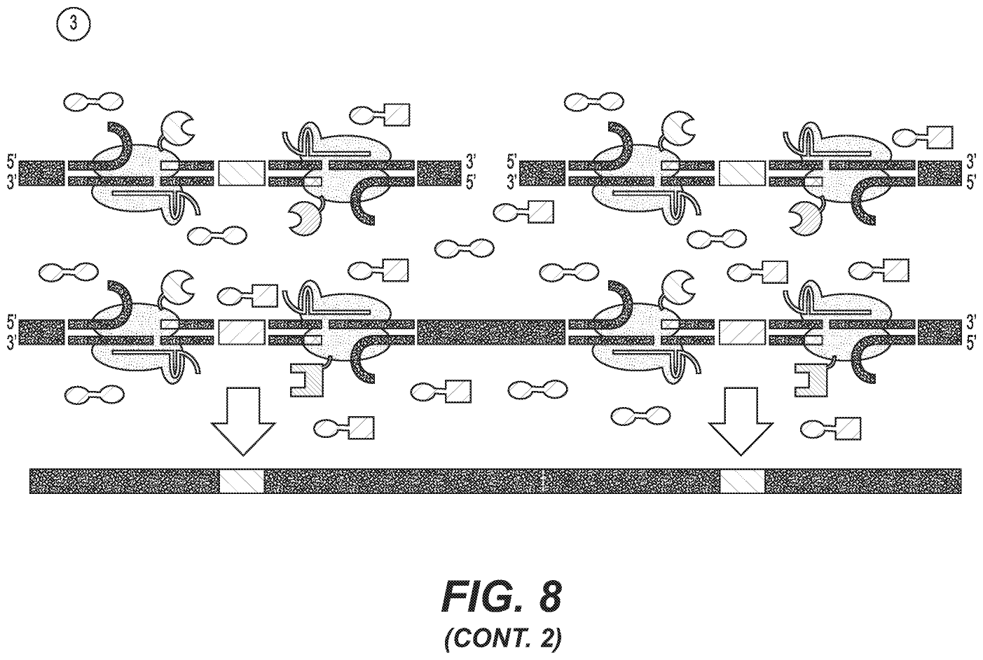

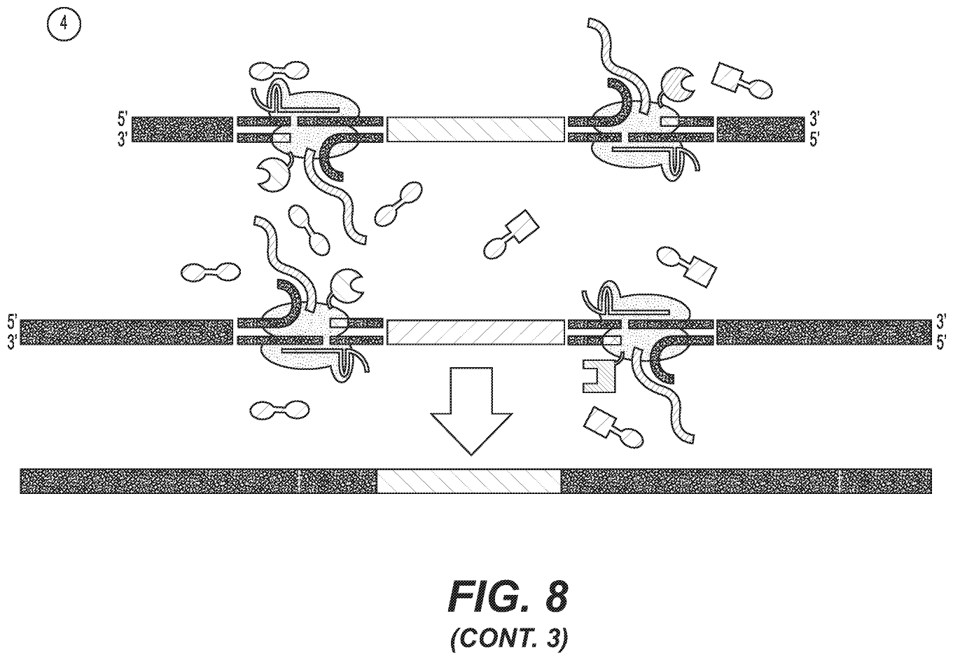

In one aspect, this application provides a third genome editing system comprising: a) a CRISPR associated (Cas) nuclease or a nucleic acid encoding the Cas nuclease, wherein the Cas nuclease is coupled to a cross-linker capable of linking two molecules of the Cas nuclease; b) a first and a second guide RNAs (gRNAs) or one or more nucleic acids encoding the first and second gRNAs, wherein a target sequence of the first gRNA and a target sequence of the second gRNA flank a target genomic segment, and wherein each of the first and second gRNAs is capable of forming a complex with the Cas nuclease; c) a template molecule flanked by a third and a fourth gRNA target sequences; and d) a first tgOligo corresponding to the first gRNA, a second tgOligo corresponding to the second gRNA, a third tgOligo corresponding to the third gRNA, and a fourth tgOligo corresponding to the fourth gRNA, wherein the first and third tgOligos are capable of hybridizing with each other, and wherein the second and fourth tgOligos are capable of hybridizing with each other. An exemplary graphic illustration is depicted in FIG. 8, panel 4. In another aspect, each end of a template molecule comprises a sequence homologous to a sequence flanking a target genomic segment.

In one aspect, this application provides a fourth genome editing system comprising: a) a CRISPR associated (Cas) nuclease or a nucleic acid encoding the Cas nuclease, wherein the Cas nuclease is coupled to a cross-linker capable of linking two molecules of the Cas nuclease; b) a first and a second guide RNAs (gRNAs) or one or more nucleic acids encoding the first and second gRNAs, wherein a target sequence of the first gRNA and a target sequence of the second gRNA flank a target genomic segment, and wherein each of the first and second gRNAs is capable of forming a complex with the Cas nuclease; c) a deactivated Cas (dCas) nuclease coupled to a cross-linker, or a nucleic acid encoding the dCas nuclease and cross-linker; and d) a third and a fourth gRNAs or one or more nucleic acids encoding the third and fourth gRNAs, wherein target sequences of the third and fourth gRNAs are within and on the opposite ends of the target genomic segment, and wherein a dCas nuclease bound to the third or fourth gRNA target sequence is capable of dimerizing with a Cas nuclease bound to a gRNA target sequence on the opposite end of the target genomic segment. An exemplary graphic illustration of this system is depicted in FIG. 6, panel 5.

In one aspect, this application provides a fifth genome editing system comprising: a) a CRISPR associated (Cas) nuclease or a nucleic acid encoding the Cas nuclease, wherein the Cas nuclease is coupled to a cross-linker capable of linking two molecules of the Cas nuclease; b) a first and a second guide RNAs (gRNAs) or one or more nucleic acids encoding the first and second gRNAs, wherein a target sequence of the first gRNA and a target sequence of the second gRNA flank a target genomic segment, and wherein each of the first and second gRNAs is capable of forming a complex with the Cas nuclease; and c) a template molecule flanked by two gRNA target sequences, wherein each end of the template molecule comprises a sequence homologous to a sequence flanking the target genomic segment. An exemplary graphic illustration is depicted in FIG. 8, panel 2. In another aspect, a fifth genome editing system further comprises multiple template molecules corresponding to multiple target genomic segments, for which an exemplary graphic illustration is depicted in FIG. 8, panel 3.

In one aspect, this application provides a sixth genome editing system comprising: a) a CRISPR associated (Cas) nuclease or a nucleic acid encoding the Cas nuclease, wherein the Cas nuclease is coupled to a cross-linker capable of linking two molecules of the Cas nuclease; b) a first and a second guide RNAs (gRNAs) or one or more nucleic acids encoding the first and second gRNAs, wherein a target sequence of the first gRNA and a target sequence of the second gRNA flank a target genomic segment, and wherein each of the first and second gRNAs is capable of forming a complex with the Cas nuclease; and c) a template molecule flanked by two gRNA target sequences, wherein each end of the template molecule comprises a sequence homologous to a sequence flanking the target genomic segment; and d) a deactivated Cas (dCas) nuclease or a nucleic acid encoding the dCas nuclease, wherein the dCas nuclease is coupled to a cross-linker and capable of being bound to the two gRNA target sequences on the template molecule. An exemplary graphic illustration is depicted in FIG. 8, panel 1. In another aspect, a sixth genome editing system comprises a dCas-coupled cross-linker capable of forming a complex with a Cas-coupled cross-linker.

In one aspect, this application provides a seventh genome editing system comprising: a) a Cas nuclease or a nucleic acid encoding the Cas nuclease; b) a first and a second gRNAs or one or more nucleic acids encoding the first and second gRNAs, wherein a target sequence of the first gRNA and a target sequence of the second gRNA flank a target genomic segment; and c) a first tgOligo corresponding to the first gRNA and a second tgOligo corresponding to second gRNA, wherein the first and second tgOligos are capable of hybridizing with each other. An exemplary graphic illustration is depicted in FIG. 6, panel 2. In another aspect, in a seventh genome editing system, target sequences of a first and a second gRNAs reside on the opposite strands of a target genomic segment.

In one aspect, this application provides an eighth genome editing system comprising: a) a Cas nuclease or a nucleic acid encoding the Cas nuclease, wherein the Cas nuclease is coupled to a cross-linker; b) a first and a second gRNAs or one or more nucleic acids encoding the first and second gRNAs, wherein a target sequence of the first gRNA and a target sequence of the second gRNA flank a target genomic segment; c) a first tgOligo corresponding to the first gRNA and a second tgOligo corresponding to second gRNA, wherein the first and second tgOligos are capable of hybridizing with each other; d) a deactivated Cas (dCas) nuclease coupled to a cross-linker, or a nucleic acid encoding the dCas nuclease and cross-linker; and e) a third and a fourth gRNAs or one or more nucleic acids encoding the third and fourth gRNAs, wherein target sequences of the third and fourth gRNAs are within and on the opposite ends of the target genomic segment; and wherein a dCas nuclease bound to the third or fourth gRNA target sequence is capable of dimerizing with a Cas nuclease bound to a gRNA target sequence on the opposite end of the target genomic segment. An exemplary graphic illustration is depicted in FIG. 6, panel 6.

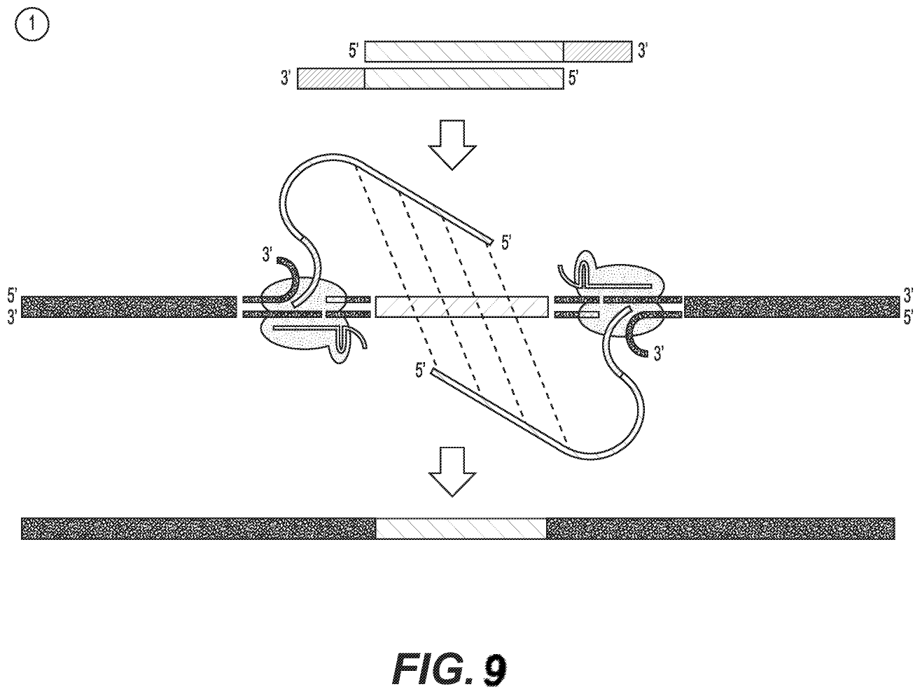

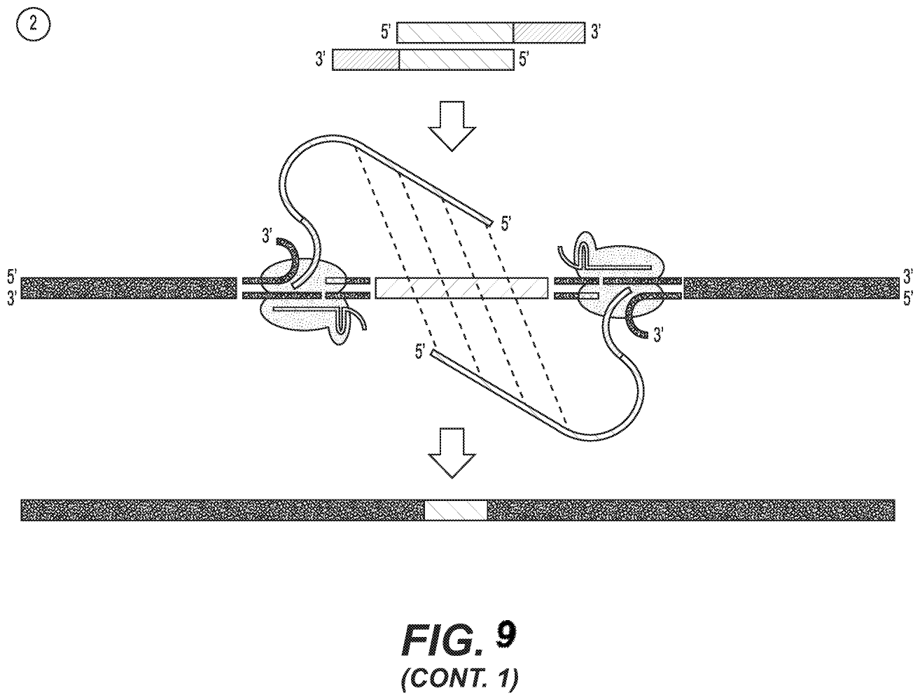

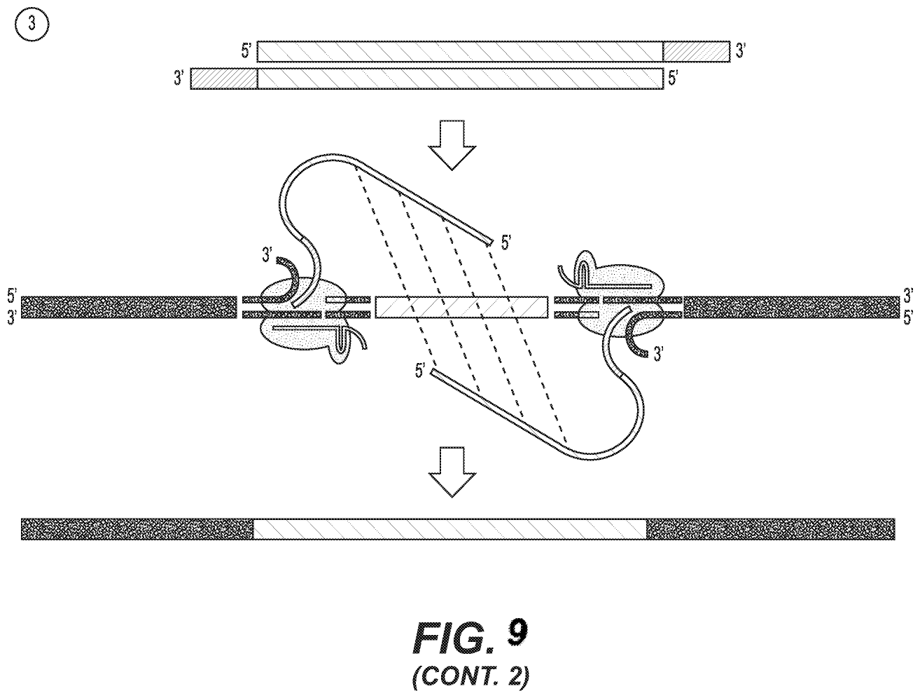

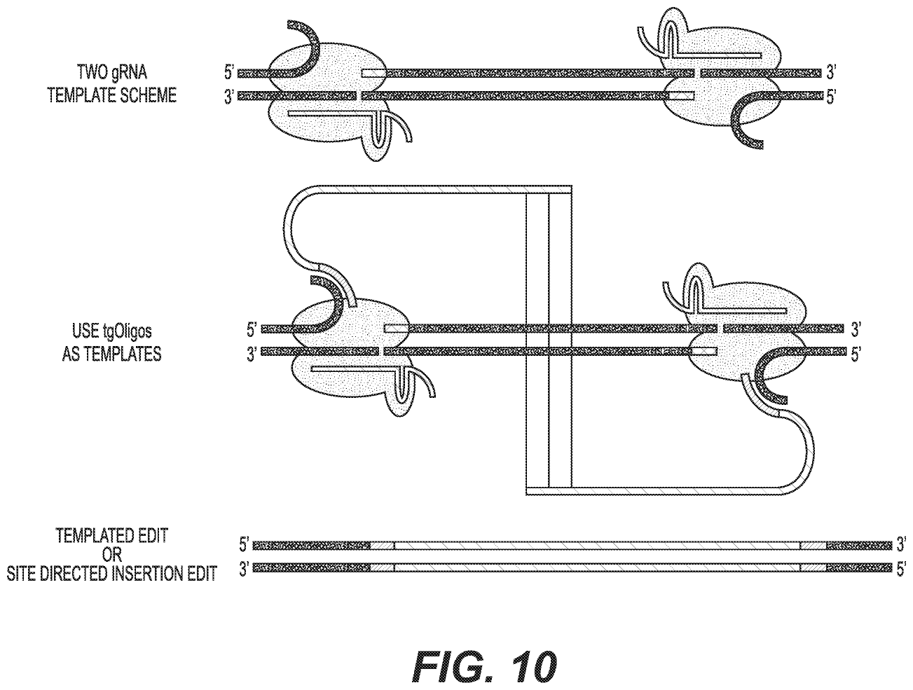

In one aspect, this application provides a ninth genome editing system comprising: a) a Cas nuclease or a nucleic acid encoding the Cas nuclease; b) a first and a second gRNAs or one or more nucleic acids encoding the first and second gRNAs, wherein a target sequence of the first gRNA and a target sequence of the second gRNA flank a target genomic segment; and c) a first tgOligo corresponding to the first gRNA and a second tgOligo corresponding to second gRNA, wherein the first and second tgOligos are capable of hybridizing with each other, wherein the first and second tgOligos are capable of hybridizing and forming a double-stranded template sequence for integration. Exemplary graphic illustrations are depicted in FIGS. 9 and 10. In one aspect, a double-stranded template sequence is capable of replacing a target genomic segment via the genome editing system. In another aspect, a double-stranded template sequence is longer, shorter, or of equal size compared to the target genomic segment.

In one aspect, this application provides a tenth genome editing system comprising: a) a Cas nuclease or a nucleic acid encoding the Cas nuclease; b) a first and a second gRNAs or one or more nucleic acids encoding the first and second gRNAs, wherein a target sequence of the first gRNA and a target sequence of the second gRNA flank a target genomic segment, c) a first tgOligo corresponding to the first gRNA and further capable of hybridizing with the target genomic segment on the opposite end of the first gRNA target site, and d) a second tgOligo corresponding to the second gRNA and further capable of hybridizing with the target genomic segment on the opposite end of the second gRNA target site. An exemplary graphic illustration is depicted in FIG. 6, panel 4. In one aspect, target sequences of a first and a second gRNAs reside on the opposite strands of a target genomic segment.

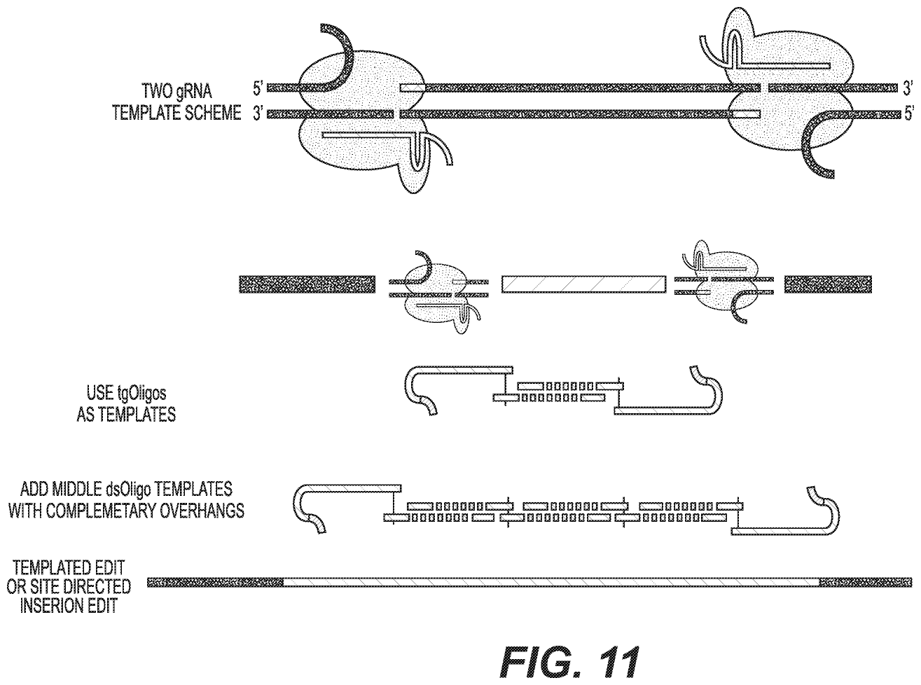

In one aspect, this application provides a eleventh genome editing system comprising: a) a Cas nuclease or a nucleic acid encoding the Cas nuclease; b) a first and a second gRNAs or one or more nucleic acids encoding the first and second gRNAs, wherein a target sequence of the first gRNA and a target sequence of the second gRNA flank a target genomic segment; c) a first tgOligo corresponding to the first gRNA and a second tgOligo corresponding to the second gRNA; d) one or more double-strand oligos (dsOligos) with two overhangs, wherein each of the two overhangs is capable of hybridizing with the first or second tgOligos. An exemplary graphic illustration is depicted in FIG. 11. In one aspect, target sequences of a first and a second gRNAs reside on the opposite strands of a target genomic segment. In another aspect, one or more dsOligos comprise a template sequence of interest or part thereof. In one aspect, one or more dsOligos comprises complementary overhangs and are capable of being integrated into a target genome as a tandem repeat.

In one aspect, a genome editing system provided here is adopted for genome editing in a plant cell. In another aspect, a genome editing system of any one of the preceding claims adopted for genome editing in a non-plant eukaryotic cell.

In one aspect, this application provides a first method for chromosome engineering comprising: introducing into a target cell a genome editing system described herein, and producing a modified chromosome comprising a deletion or inversion of the target genomic segment or a replacement of the target genomic segment based on the template molecule.

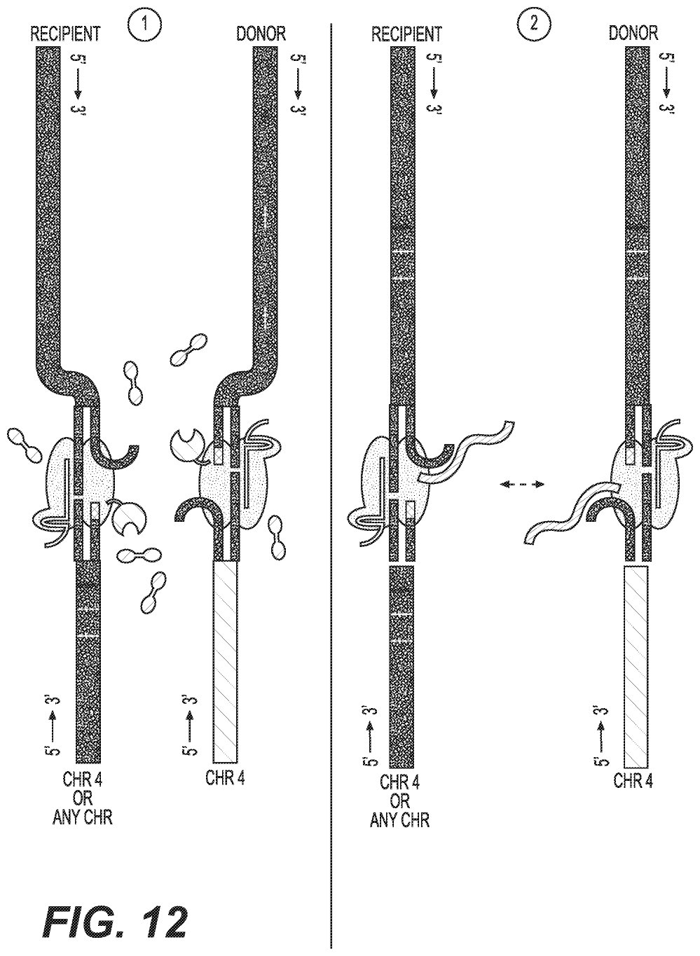

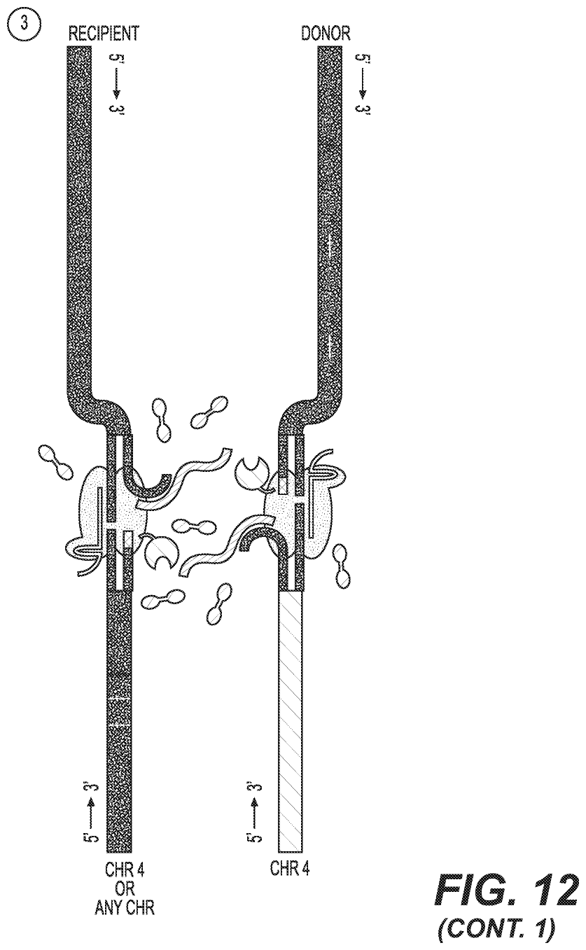

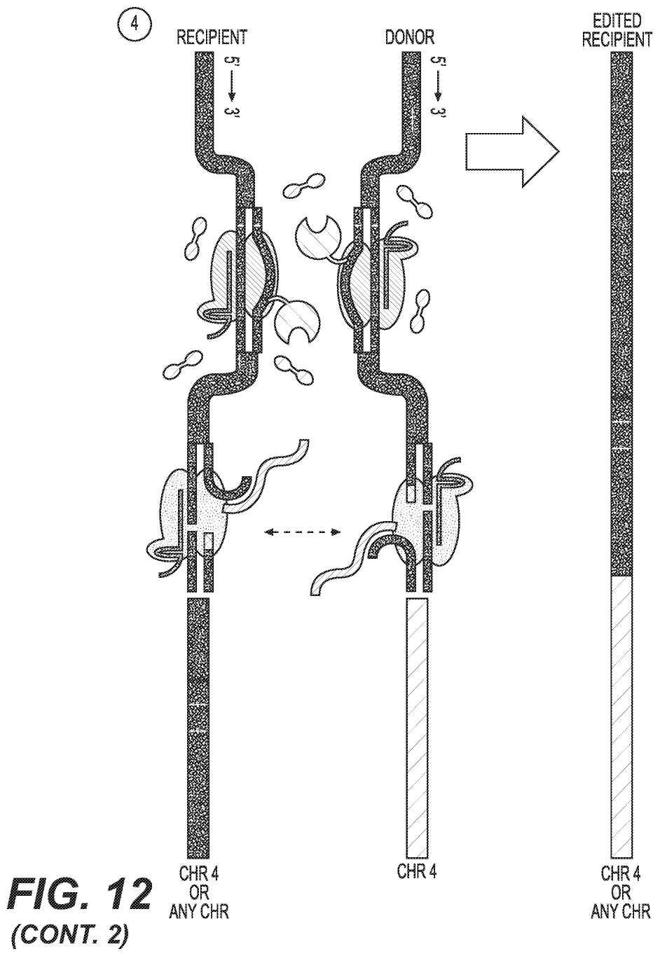

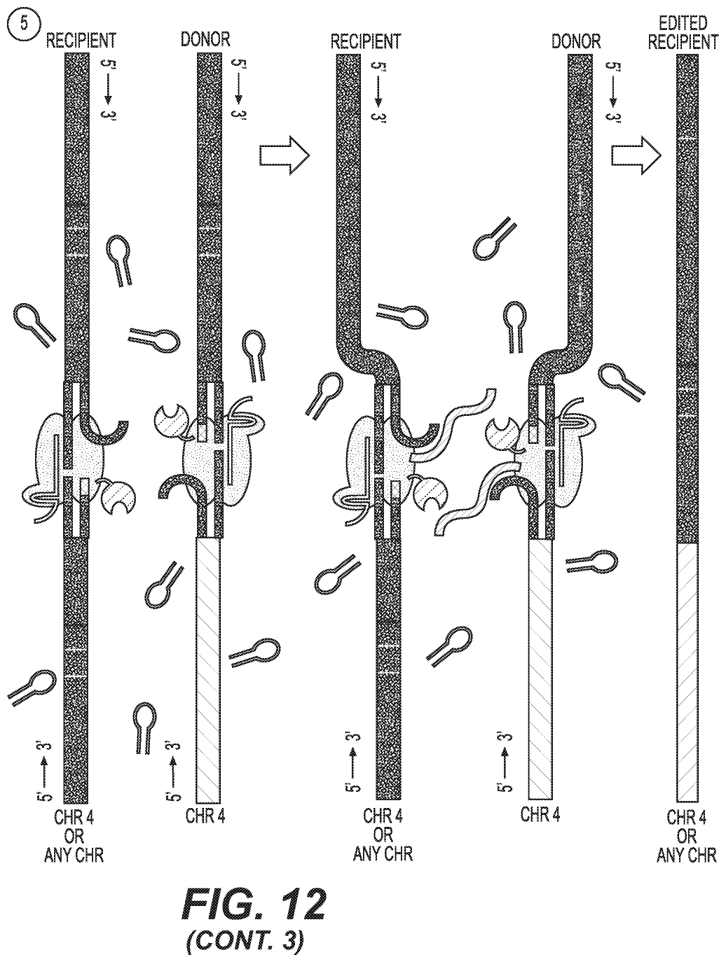

In one aspect, this application provides a second method for chromosome engineering comprising: introducing into a target cell a genome editing system comprising: a) a Cas nuclease coupled to a cross-linker or a nucleic acid encoding the Cas nuclease and cross-linker, wherein the cross-linker is capable of linking two molecules of the Cas nuclease; and b) a first and a second gRNAs or one or more nucleic acids encoding the first and second gRNAs, and wherein the first and second gRNAs have target sequences in a first recombination region of interest on a pair of donor and recipient chromosomes; and producing a recombinant chromosome comprising a portion of the donor chromosome and a portion of the recipient chromosome. An exemplary graphic illustration is depicted in FIG. 12, panel 1. In one aspect, target sequences of a first and a second gRNAs reside in a homologous region of the pair of donor and recipient chromosomes. In another aspect, a cross-linker is capable of linking two molecules of the Cas nuclease bound to the target sequences of the first and second gRNAs. In one aspect, a cross-linker is capable of linking two molecules of the Cas nuclease to increase recombination frequency in the first recombination region of interest. In another aspect, a first and a second gRNAs are identical. In another aspect, a cross-linker is a homo-dimerization domain. In one aspect, a cross-linker is a hetero-dimerization domain. In another aspect, a cross-linker requires a cross-linking ligand. In one aspect, a cross-linker is an inducible dimerization domain. In another aspect, a cross-linker is a single-strand DNA or RNA binding domain.

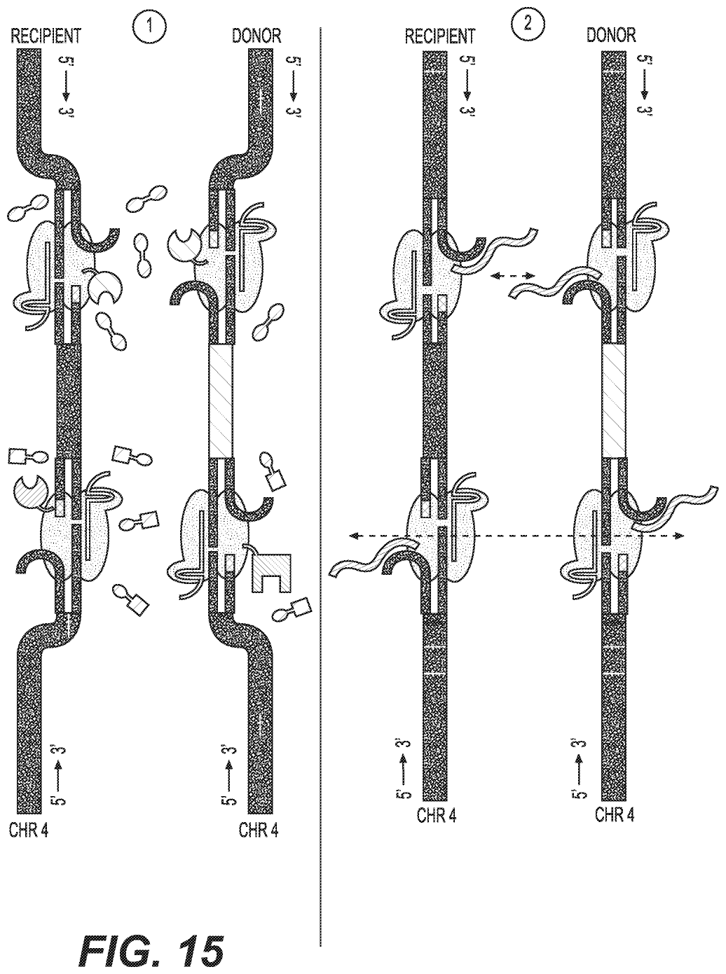

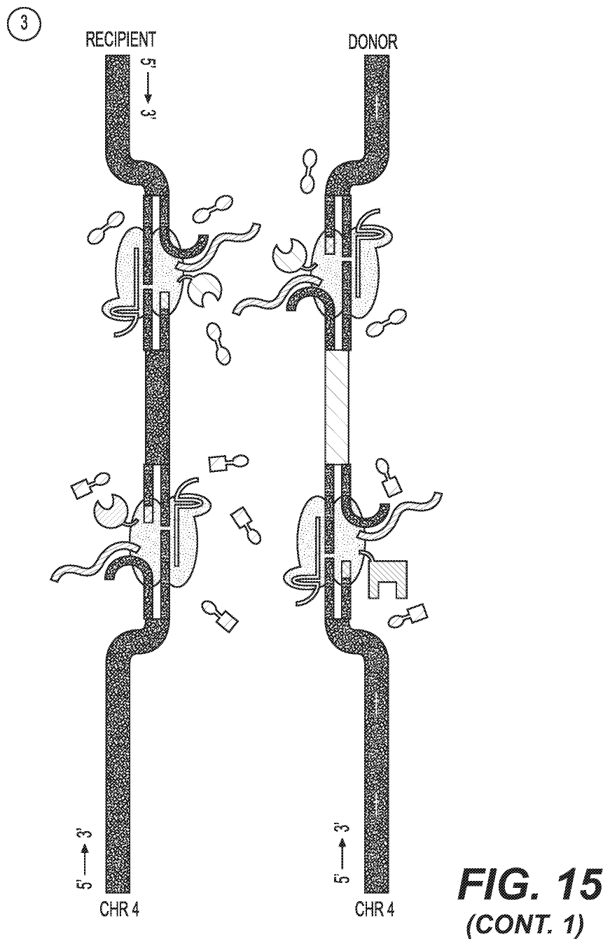

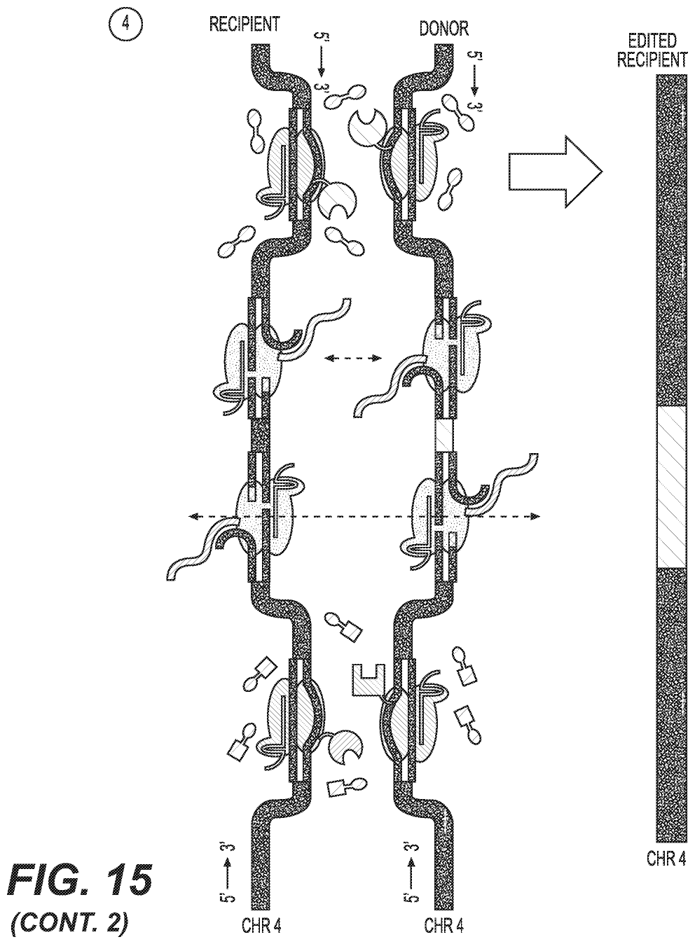

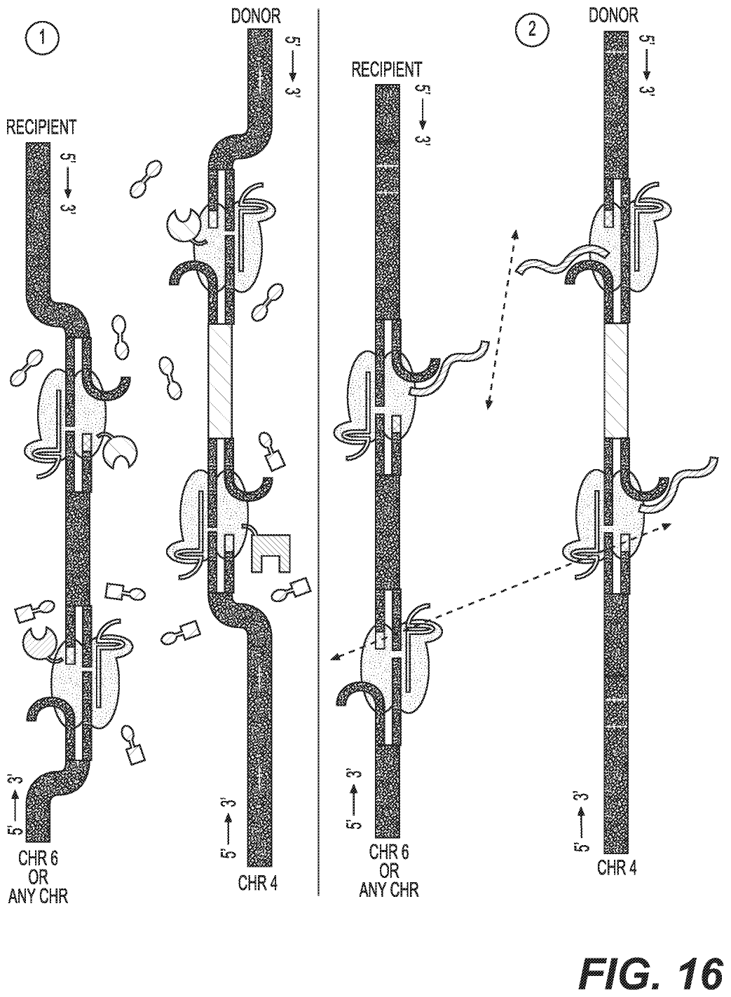

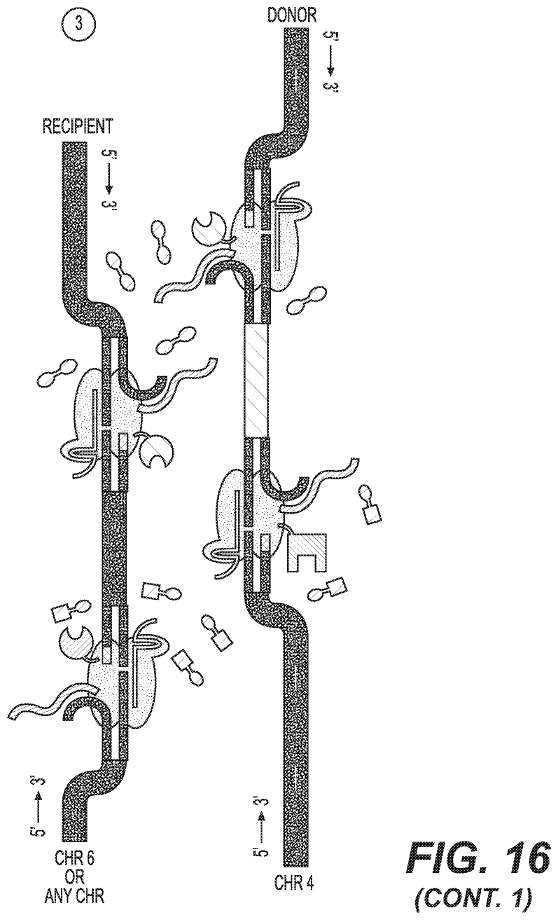

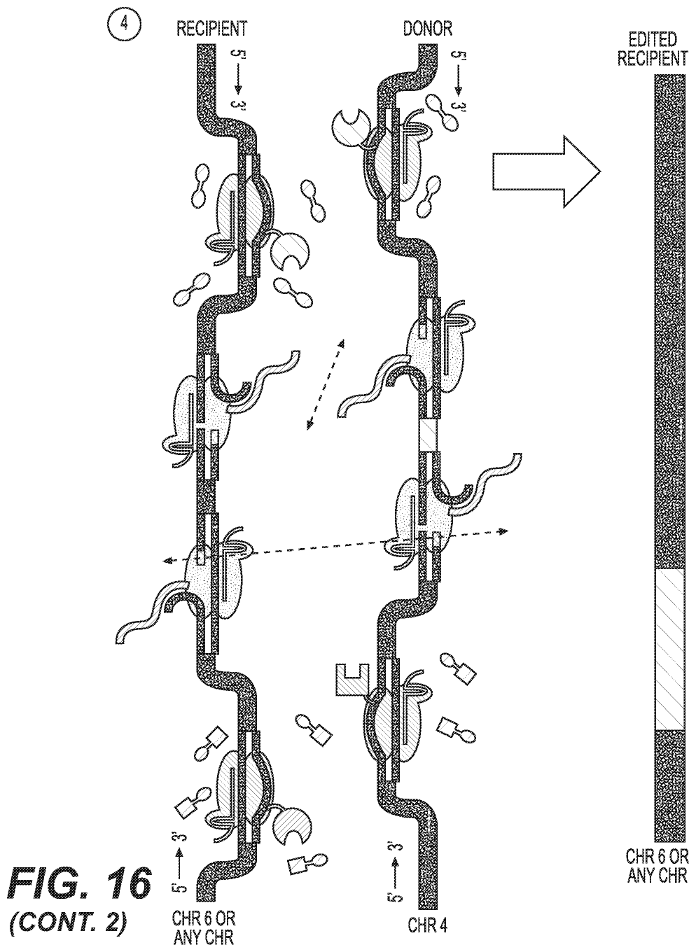

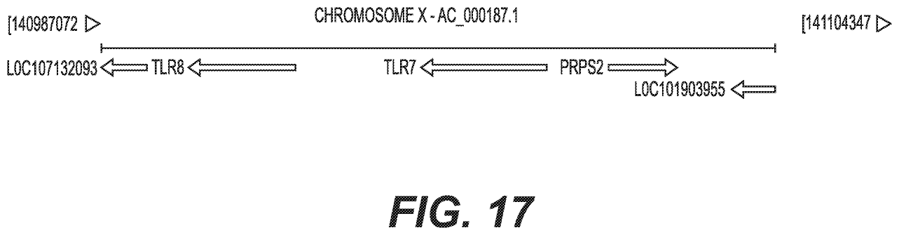

In one aspect, this application provides a third method for chromosome engineering comprising: introducing into a target cell a genome editing system comprising: a) a Cas nuclease coupled to a cross-linker or a nucleic acid encoding the Cas nuclease and cross-linker, wherein the cross-linker is capable of linking two molecules of the Cas nuclease; b) a first and a second gRNAs or one or more nucleic acids encoding the first and second gRNAs, and wherein the first and second gRNAs have target sequences in a first recombination region of interest on a pair of donor and recipient chromosomes; and c) a third and a fourth gRNAs or one or more nucleic acids encoding the third and fourth gRNAs, and wherein the third and fourth gRNAs have target sequences in a second recombination region of interest on the pair of donor and recipient chromosomes; and producing a recombinant chromosome comprising a portion of the donor chromosome and a portion of the recipient chromosome, wherein the method is capable of producing a recombinant chromosome comprising a backbone from the recipient chromosome with a chromosome segment integrated from the donor chromosome between the first and second recombination regions of interest. An exemplary graphic illustration is depicted in FIG. 15, panel 1. In one aspect, a pair of donor and recipient chromosomes are homologous chromosomes. In another aspect, a pair of donor and recipient chromosomes are non-homologous chromosomes. An exemplary graphic illustration is depicted in FIG. 16, panel 1.

In one aspect, this application provides a fourth method for chromosome engineering comprising: introducing into a target cell a genome editing system comprising: a) a Cas nuclease coupled to a cross-linker or a nucleic acid encoding the Cas nuclease and cross-linker, wherein the cross-linker is capable of linking two molecules of the Cas nuclease; b) a first and a second gRNAs or one or more nucleic acids encoding the first and second gRNAs, and wherein the first and second gRNAs have target sequences in a first recombination region of interest on a pair of donor and recipient chromosomes; and c) a first tgOligo corresponding to the first gRNA, a second tgOligo corresponding to the second gRNA, and wherein the first and second tgOligos are capable of hybridizing with each other; and producing a recombinant chromosome comprising a portion of the donor chromosome and a portion of the recipient chromosome. An exemplary graphic illustration is depicted in FIG. 12, panel 3. In another aspect, a genome editing system used in the fourth method further comprises: d) a third and a fourth gRNAs or one or more nucleic acids encoding the third and fourth gRNAs, and wherein the third and fourth gRNAs have target sequences in a second recombination region of interest on the pair of donor and recipient chromosomes; e) a third tgOligo corresponding to the third gRNA, a fourth tgOligo corresponding to the fourth gRNA, and wherein the third and fourth tgOligos are part of a single molecule or are capable of hybridizing with each other; wherein the method is capable of producing a recombinant chromosome comprising a backbone from the recipient chromosome with a chromosome segment integrated from the donor chromosome between the first and second recombination regions of interest. An exemplary graphic illustration is depicted in FIG. 15, panel 3. In one aspect, a pair of donor and recipient chromosomes are homologous chromosomes. In another aspect, a pair of donor and recipient chromosomes are non-homologous chromosomes. An exemplary graphic illustration is depicted in FIG. 16, panel 3.

In one aspect, a genome editing system used in a third or a fourth method further comprises: f) a deactivated Cas (dCas) nuclease coupled to a cross-linker, or a nucleic acid encoding the dCas nuclease and cross-linker; g) a third and a fourth gRNAs or one or more nucleic acids encoding the third and fourth gRNAs, wherein a target sequence of the third gRNA and a target sequence of the fourth gRNA each reside on one chromosome of the pair of donor and recipient chromosomes, wherein two cross-linked molecules of the dCas nuclease are capable of binding to the third and fourth gRNA target sequences and thereby bringing into close proximity the first recombination region of interest and promoting recombination. An exemplary graphic illustration is depicted in FIG. 12, panel 4.

In one aspect, a genome editing system used a third or a fourth method further comprises: h) a fifth and a sixth gRNAs or one or more nucleic acids encoding the fifth and sixth gRNAs, and wherein the fifth and sixth gRNAs have target sequences in a second recombination region of interest on the pair of donor and recipient chromosomes; and i) a seventh and a eighth gRNAs or one or more nucleic acids encoding the seventh and eighth gRNAs, wherein a target sequence of the seventh gRNA and a target sequence of the eighth gRNA each reside on one chromosome of the pair of donor and recipient chromosomes, wherein two cross-linked molecules of the dCas nuclease are capable of binding to the seventh and eighth gRNA target sequences and thereby bringing into close proximity the second recombination region of interest and promoting recombination; wherein the method is capable of producing a recombinant chromosome comprising a backbone from the recipient chromosome with a chromosome segment integrated from the donor chromosome between the first and second recombination regions of interest. A graphic illustration is depicted in FIG. 15, panel 4. In one aspect, a pair of donor and recipient chromosomes are homologous chromosomes. In another aspect, a pair of donor and recipient chromosomes are non-homologous chromosomes. An exemplary graphic illustration is depicted in FIG. 16, panel 4. In a further aspect, a donor or a recipient chromosome is a supernumerary/B chromosome.

In one aspect, this application provides a fifth method for chromosome engineering comprising: introducing into a target cell a genome editing system comprising: a) a Cas nuclease or a nucleic acid encoding the Cas nuclease; b) a first and a second gRNAs or one or more nucleic acids encoding the first and second gRNAs, and wherein the first and second gRNAs have target sequences in a first recombination region of interest on a pair of donor and recipient chromosomes; and c) a first tgOligo corresponding to the first gRNA, a second tgOligo corresponding to the second gRNA, and wherein the first and second tgOligos are part of a single molecule or are capable of hybridizing with each other; and producing a recombinant chromosome comprising a portion of the donor chromosome and a portion of the recipient chromosome. An exemplary graphic illustration is depicted in FIG. 12, panel 2. In one aspect, a first, a second, or both tgOligos comprise a hairpin configuration until a portion of the tgOligo sequence hybridizes with an intended genomic sequence. In another aspect, a non-hybridized portion of the first, second, or both tgOligos unfold into a single-strand form upon the hybridization.

In one aspect, a genome editing system used in a fifth method further comprises: f) a third and a fourth gRNAs or one or more nucleic acids encoding the third and fourth gRNAs, and wherein the third and fourth gRNAs have target sequences in a second recombination region of interest on the pair of donor and recipient chromosomes; and g) a third tgOligo corresponding to the third gRNA, a fourth tgOligo corresponding to the fourth gRNA, and wherein the third and fourth tgOligos are part of a single molecule or are capable of hybridizing with each other; and wherein the method is capable of producing a recombinant chromosome comprising a backbone from the recipient chromosome with a chromosome segment integrated from the donor chromosome between the first and second recombination regions of interest. An exemplary graphic illustration is depicted in FIG. 15, panel 2. In one aspect, a pair of donor and recipient chromosomes are homologous chromosomes. In another aspect, a pair of donor and recipient chromosomes are non-homologous chromosomes. An exemplary graphic illustration is depicted in FIG. 16, panel 2.

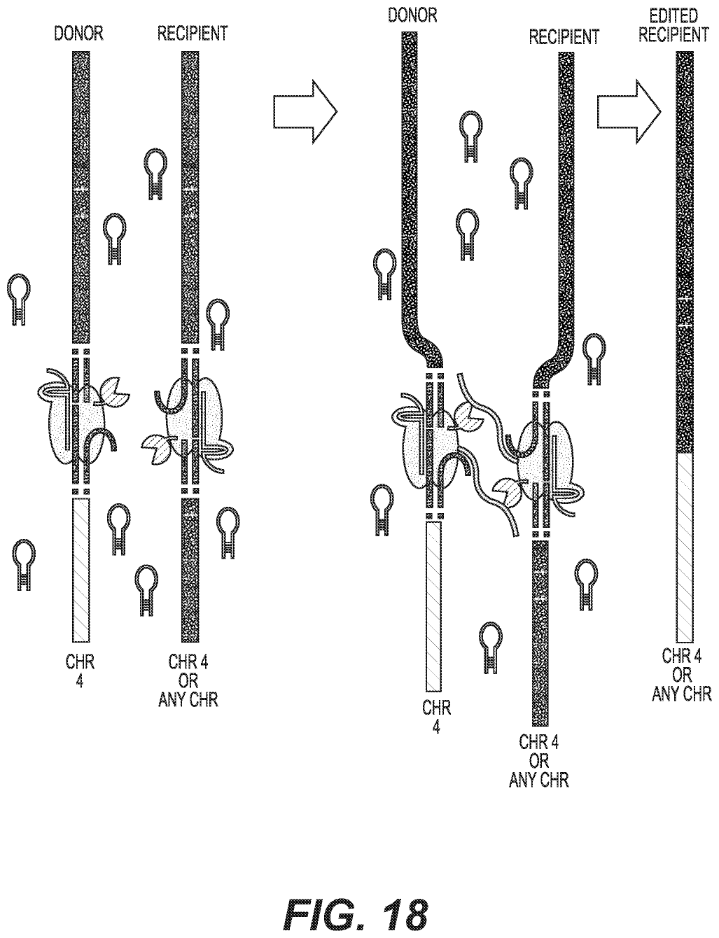

In one aspect, this application provides a sixth method for chromosome engineering comprising: introducing into a target cell a genome editing system comprising: a) a Cas nuclease coupled to a single-strand nucleic acid-binding domain heterologous to the Cas nuclease or a nucleic acid encoding the Cas nuclease and the single-strand nucleic acid-binding domain, b) a first and a second gRNAs or one or more nucleic acids encoding the first and second gRNAs, wherein the first and second gRNAs have target sequences in a first recombination region of interest on a pair of donor and recipient chromosomes, c) a first tgOligo corresponding to the first gRNA and a second tgOligo corresponding to the second gRNA, wherein the first, second, or both tgOligos comprise a hairpin configuration until a portion of the tgOligo sequence hybridizes with an intended genomic sequence, and wherein the non-hybridized portion of the first, second, or both tgOligos unfolds into a single-strand form upon the hybridization and further binds the single-strand nucleic acid-binding domain; producing a recombinant chromosome comprising a portion of the donor chromosome and a portion of the recipient chromosome. An exemplary graphic illustration is depicted in FIG. 12, panel 5 and FIG. 18. In one aspect, target sequences of a first and a second gRNAs reside in a homologous region of the pair of donor and recipient chromosomes. In one aspect, a pair of donor and recipient chromosomes are homologous chromosomes. In another aspect, a pair of donor and recipient chromosomes are non-homologous chromosomes. An exemplary graphic illustration is depicted in FIG. 16, panel 2.

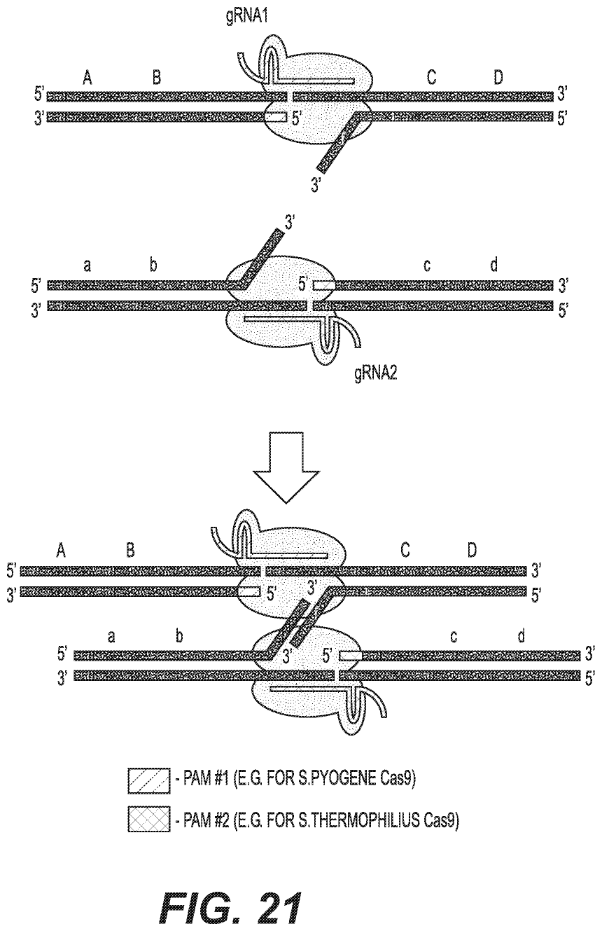

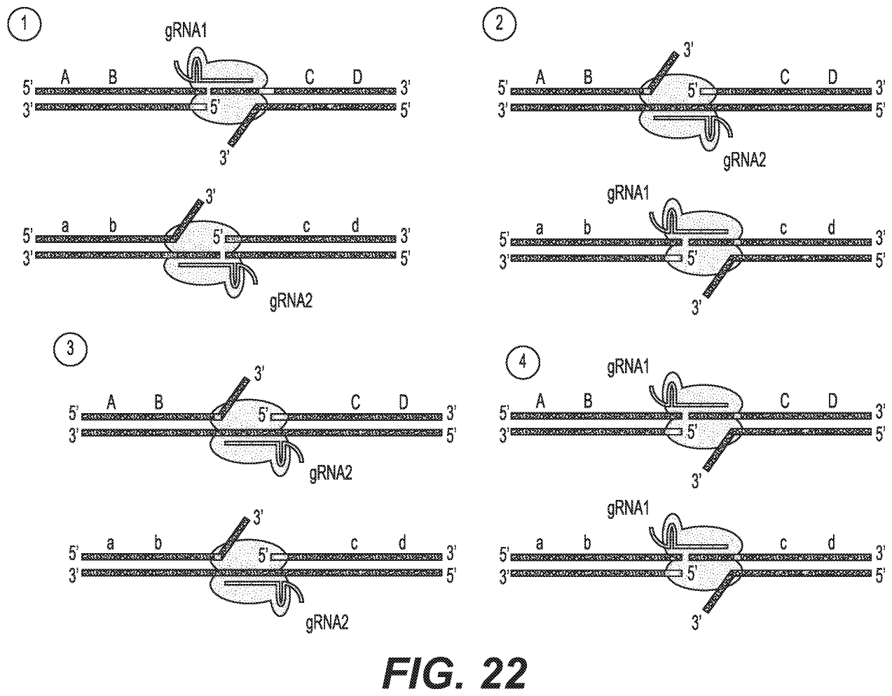

In one aspect, this application further provides a twelfth genome editing system comprising: a) a CRISPR associated (Cas) nuclease or a nucleic acid encoding the Cas nuclease; and b) a first and a second guide RNAs (gRNAs) or one or more nucleic acids encoding the first and second gRNAs, wherein the first and second gRNAs have target sequences arranged such that the double-strand DNA cleavage mediated by the first and second gRNAs is capable of creating two 3′ free ends from non-target strands complementing each other. Exemplary graphic illustrations are depicted in FIG. 21 and FIG. 22. In one aspect, a first and a second gRNAs recognize two different Cas nucleases. In another aspect, two different Cas nucleases are from two species selected from the group consisting of Streptococcus pyogenes, Streptococcus thermophilius, Staphylococcus aureus, Neisseria meningitides, and Treponema denticola. In a further aspect, two different Cas nucleases are from Streptococcus pyogenes and Streptococcus thermophilius, respectively. In another aspect, a first and a second gRNAs have two different PAM sequences.

In one aspect, this application further provides a method for chromosome engineering comprising: introducing into a target cell a genome editing system comprising: a) a first and a second CRISPR associated (Cas) nucleases or one or more nucleic acids encoding the first and second Cas nucleases, and b) a first and a second guide RNAs (gRNAs) or one or more nucleic acids encoding the first and second gRNAs, wherein the first and second gRNAs are binding with the first and second Cas nucleases which mediate double-strand DNA cleavage, wherein the first and second gRNAs have target sequences arranged such that the double-strand DNA cleavage is capable of creating two 3′ free ends from non-target strands complementing each other, and wherein the first and second gRNA target sequences are in a recombination region of interest on a pair of donor and recipient chromosomes; and producing a recombinant chromosome comprising a portion of the donor chromosome and a portion of the recipient chromosome. Exemplary graphic illustrations of this aspect are depicted in FIG. 21 and FIG. 22.

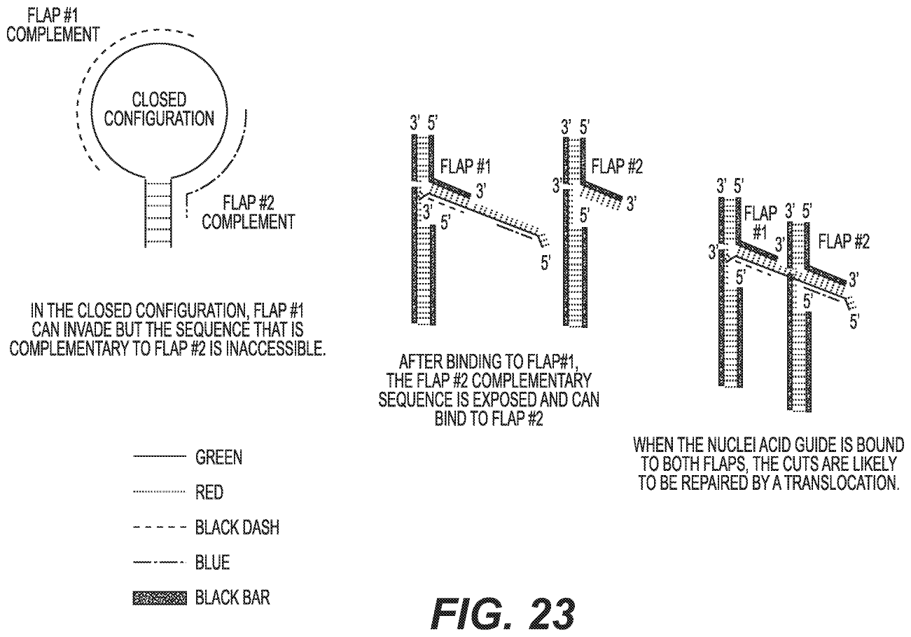

In one aspect, this application provides a thirteen genome editing system comprising: a) a CRISPR associated (Cas) nuclease or a nucleic acid encoding the Cas nuclease, b) a first and a second guide RNAs (gRNAs) or one or more nucleic acids encoding the first and second gRNAs, c) a chimeric tgOligo comprising sequences capable of recognizing the target sites of both the first and second gRNAs and binding both non-target strand 3′ free ends generated from DNA cleavage mediated by the Cas nuclease. An exemplary graphic illustration is depicted in FIG. 23. In one aspect, a chimeric tgOligo comprises a hairpin configuration until a portion of the tgOligo sequence hybridizes with an intended genomic sequence. In another aspect, a first and a second gRNAs recognize two different Cas nucleases. In one aspect, two different Cas nucleases are from two species selected from the group consisting of Streptococcus pyogenes, Streptococcus thermophilius, Staphylococcus aureus, Neisseria meningitides, and Treponema denticola. In another aspect, the two different Cas nucleases are from Streptococcus pyogenes and Streptococcus thermophilius, respectively. In one aspect, a first and a second gRNAs have two different PAM sequences.

In one aspect, this application further provides a method for chromosome engineering comprising: introducing into a target cell a thirteenth genome editing system described above, wherein a first and a second gRNA target sequences are in a recombination region of interest on a pair of donor and recipient chromosomes, and producing a recombinant chromosome comprising a portion of the donor chromosome and a portion of the recipient chromosome. In one aspect, a pair of donor and recipient chromosomes are homologous chromosomes. In another aspect, a pair of donor and recipient chromosomes are non-homologous chromosomes.

In one aspect, a method for genome editing or chromosome engineering disclosed herein is for increasing the recovery rate of desired genomic segment inversions. In another aspect, a method for genome editing or chromosome engineering disclosed herein is for facilitating site directed integration (SDI). In one aspect, a method for genome editing or chromosome engineering disclosed herein is for facilitating large site directed integration (SDI). In another aspect, a method for genome editing or chromosome engineering disclosed herein is for creating chromosome exchanges and deletions. In one aspect, a method for genome editing or chromosome engineering disclosed herein is for facilitating cis chromosome arm exchange.

In another aspect, this application also provides one or more recombinant constructs, vectors, or plasmids that encode a genome editing system described herein. Further provided are host cells (e.g., bacterial cell, plant cell, or mammalian cells) that harbors such constructs, vectors, or plasmids. In another aspect, a cell targeted for genome engineering is transformed or transfected with one or more genome editing system described herein. In another aspect, a modified cell with desired genome edits or recombination is selected and obtained by using one or more genome editing system described herein.

### Definitions

Unless defined otherwise, technical and scientific terms used herein have the same meaning as commonly understood by one of ordinary skill in the art. One skilled in the art will recognize many methods can be used in the practice of the present disclosure. Indeed, the present disclosure is in no way limited to the methods and materials described. For purposes of the present disclosure, the following terms are defined below.

As used herein, the singular form “a,” “an,” and “the” include plural references unless the context clearly dictates otherwise. For example, the term “a compound” or “at least one compound” can include a plurality of compounds, including mixtures thereof.

The term “and/or” when used in a list of two or more items, means that any one of the listed items can be employed by itself or in combination with any one or more of the listed items. For example, the expression “A and/or B” is intended to mean either or both of A and B—e.g., A alone, B alone, or A and B in combination. The expression “A, B and/or C” is intended to mean A alone, B alone, C alone, A and B in combination, A and C in combination, B and C in combination, or A, B, and C in combination.

As used herein, a “nuclease” refers to a protein capable of introducing a double strand break into a DNA sequence.

As used herein, a “DNA-targeting guide molecule” refers to a molecule capable of recognizing a specific target DNA sequence and guiding another desired molecular component (e.g., a separate Cas nuclease molecule, or a FokI nuclease conjugated to a guide molecule) to the target DNA sequence for an intended action (e.g., DNA cleavage).

As used herein, a “tether molecule” refers to a molecule capable of tethering two or more DNA-binding machineries comprised of a nuclease component and a DNA-targeting guide molecule component. As used herein, two molecules are tethered together if the relative movement between these two molecules is restricted.

As used herein, a “cross-linker” refers to a molecular moiety or protein domain capable of linking two desired molecules together via non-covalent bonding.

As used herein, a CRISPR associated (“Cas”) nuclease refers to a protein encoded by a gene generally coupled, associated or close to or in the vicinity of flanking CRISPR loci, and further capable of introducing a double strand break into a DNA target sequence. A Cas nuclease is guided by a guide polynucleotide to recognize and optionally introduce a double strand break at a specific target site into the genome of a cell. Upon recognition of a target sequence by a guide RNA, a Cas nuclease unwinds the DNA duplex in close proximity of the target sequence and cleaves both DNA strands, but only if the correct protospacer-adjacent motif (PAM) is approximately oriented at the 3′ end of the target sequence.

As used herein, a “guide RNA” (gRNA) refers to a RNA molecule having a synthetic sequence and typically comprising two sequence components: a gRNA spacer sequence (also called guide sequence) and a gRNA scaffold sequence. These two sequence components can be in a single RNA molecule (also known as single-chain guide RNA (sgRNA)) or in a double-RNA molecule configuration (also known as a duplex guide RNA which comprises both a CRISPR RNA (crRNA) and a trans-activating crRNA (tracrRNA)). In some instances, a gRNA can have a crRNA component only (without a tracrRNA), for example, gRNAs that work with Cpf1). In some embodiments, a CRISPR associate protein as described herein may utilize a guide nucleic acid comprising DNA, RNA or a combination of DNA and RNA. The term “guide nucleic acid” is inclusive, referring both to double-molecule guides and to single-molecule guides.

As used herein, a gRNA “spacer sequence” or “guide sequence” refers to a RNA sequence that complements and anneals with one DNA strand of a CRISPR DNA target site via RNA-DNA pairing, which strand is called target strand. The other strand that do not hybridize with the gRNA spacer sequence is called non-target strand.

As used herein, a gRNA “scaffold sequence” refers to a sequence within a gRNA that is responsible for Cas9 binding.

As used herein, a “target site” of a CRISPR complex refers to a genomic site or DNA locus capable of being recognized by and bound to a CRISPR gRNA-Cas complex. An enzymatically active CRISPR gRNA-Cas complex would process such a target site to result in a double-strand break at the CRISPR target site. In the case of a deactivated Cas, a gRNA-dCas still recognizes and binds a CRISPR target site without cutting the target DNA.

As used herein, a “target sequence” of a CRISPR complex refers to a sequence to which a guide sequence is designed to have complementarity, where hybridization between a target sequence and a guide sequence promotes the formation of a CRISPR complex.

As used herein, a “tether guide oligo” (tgOligo) refers to an oligonucleotide comprising a sequence segment capable of hybridizing with the 3′ free end of the non-target strand of a double-stranded DNA molecule recognized and cleaved by a CRISPR gRNA-Cas complex (this 3′ free end is also referred to as 3′ free flap). A tgOligo corresponds to a gRNA when that tgOligo recognizes and hybridizes the 3′ free end of the non-target strand of that gRNA's target site. A tgOligo can be a DNA molecule, a RNA molecule, or a mix of nucleotides. A hybrid tgOligo is a tgOligo that can recognize and hybridize with two non-target 3′ free ends created by two separate CRISPR gRNA-Cas complexes.

As used herein, a “tether guide RNA” (tgRNA) refers to a RNA molecule comprising both a guide RNA (gRNA) sequence and a tether RNA sequence, where the tether RNA sequence is capable of hybridizing with a desired genomic site (which site is called “tether site”).

As used herein, a “protospacer adjacent motif” (PAM) refers to a 2-6 base pair DNA sequence immediately following a target sequence of a CRISPR complex.

As used herein, a “DNA cut” refers to a DNA double-strand break.

As used herein, a “multi-unit complex” refers to a protein or protein-nucleic acid complex comprising multiple components that are held together via non-covalent bond-mediated interaction.

As used herein, a “single molecule” refers to a single continuous molecule, the formation of which involves only covalent bonds.

As used herein, a “deactivated Cas nuclease” (dCas) refers to a nuclease comprising a domain that retains the ability to bind its target nucleic acid but has a diminished, or eliminated, ability to cleave a nucleic acid molecule, as compared to a control nuclease. In an aspect, a catalytically inactive nuclease is derived from a “control” or “wild type” nuclease. As used herein, a “control” nuclease refers to a naturally-occurring nuclease that can be used as a point of comparison for a catalytically inactive nuclease. In some embodiments, the catalytically inactive nuclease is a catalytically inactive Cas9. In some embodiments, the catalytically inactive Cas9 produces a nick in the targeting strand. In some embodiments, the catalytically inactive Cas9 comprises an Alanine substitution of key residues in the RuvC domain (D10A). In some embodiments, the catalytically inactive Cas9 produces a nick in the nontargeting strand. In some embodiments, the catalytically inactive Cas9 comprises a H840A mutation of the HNH domain. In some embodiments, the catalytically inactive Cas9, known as dead Cas9 (dCas9), lacks all nuclease activity. In some embodiments, the catalytically inactive Cas9 comprises both D10A/H840A mutations. In some embodiments, the catalytically inactive nuclease is a catalytically inactive Cpf1 (also known as Cas12a). In some embodiments, the catalytically inactive Cpf1 produces a nick in the targeting strand. In some embodiments, the catalytically inactive Cpf1 produces a nick in the nontargeting strand. In some embodiments, the catalytically inactive Cpf1, known as dead Cpf1 (dCpf1), lacks all DNase activity. In some embodiments, the catalytically inactive Cpf1 comprises a R1226A mutation in the Nuc domain. In some embodiments, the catalytically inactive Cpf1 comprises an E993A mutation in the RuvC domain, wherein the DNase activities against both strands of target DNA is eliminated. In some embodiments, the catalytically inactive Cpf1 is a dead Cpf1 endonuclease from Acidaminococcus sp. BV3L6 (dAsCpf1).

As used herein, a “donor chromosome” refers to a chromosome comprising and providing a sequence of interest that is to be translocated to another chromosomal position.

As used herein, a “recipient chromosome” refers to a chromosome that will receive a a sequence of interest upon chromosome engineering.

The practice of the present disclosure employs, unless otherwise indicated, techniques of biochemistry, chemistry, molecular biology, microbiology, cell biology, genomics and biotechnology, which are within the skill of the art. See Green and Sambrook, Molecular Cloning: A Laboratory Manual, 4th Edition (2012); Current Protocols In Molecular Biology (F. M. Ausubel, et al. eds., (1987)); the series Methods In Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Animal Cell Culture (R. I. Freshney, ed. (1987)); Recombinant Protein Purification: Principles And Methods, 18-1142-75, GE Healthcare Life Sciences; C. N. Stewart, A. Touraev, V. Citovsky, T. Tzfira eds. (2011) Plant Transformation Technologies (Wiley-Blackwell); and R. H. Smith (2013) Plant Tissue Culture. Techniques And Experiments (Academic Press, Inc.).

Any references cited herein, including, e.g., all patents, published patent applications, and non-patent publications, are hereby incorporated by reference in their entirety.

Nucleic acid molecules mentioned herein include, without limitation, deoxyribonucleic acids (DNA) and ribonucleic acids (RNA) and functional analogues thereof, such as complementary DNA (cDNA). Nucleic acid molecules provided herein can be single stranded or double stranded. Nucleic acid molecules comprise the nucleotide bases adenine (A), guanine (G), thymine (T), cytosine (C). Uracil (U) replaces thymine in RNA molecules. The symbol “N” can be used to represent any nucleotide base (e.g., A, G, C, T, or U). As used herein, “encoding” refers to a polynucleotide encoding for the amino acids of a polypeptide or a non-coding RNA molecule. A series of three nucleotide bases encodes one amino acid. As used herein, “expressed,” “expression,” or “expressing” refers to transcription of RNA from a DNA molecule. As used herein, terms “polypeptide”, “peptide” and “protein” are used interchangeably to refer to a polymer of amino acid residues. The term also applies to amino acid polymers in which one or more amino acids are chemical analogues or modified derivatives of a corresponding naturally-occurring amino acids. A “messenger RNA” or “mRNA” refers to an RNA transcript that is transcribed from a polynucleotide, where the RNA transcript is capable of being translated into a protein. Typically, DNA encodes an mRNA, which encodes a protein or a non-coding RNA molecule. When DNA is transcribed by an RNA polymerase to ultimately generate a protein, a sense mRNA strand is typically produced by the RNA polymerase from the antisense DNA strand.

As used herein, the term “operably linked” refers to a functional linkage between a promoter or other regulatory element and an associated transcribable DNA sequence or coding sequence of a gene (or transgene), such that the promoter, etc., operates to initiate, assist, affect, cause, and/or promote the transcription and expression of the associated transcribable DNA sequence or coding sequence, at least in certain tissue(s), developmental stage(s) and/or condition(s). In addition to promoters, regulatory elements include, without being limiting, an enhancer, a leader, a transcription start site (TSS), a linker, 5′ and 3′ untranslated regions (UTRs), an intron, a polyadenylation signal, and a termination region or sequence, etc., that are suitable, necessary or preferred for regulating or allowing expression of the gene or transcribable DNA sequence in a cell. Such additional regulatory element(s) can be optional and used to enhance or optimize expression of the gene or transcribable DNA sequence.

As used herein, the term “promoter” refers to a DNA sequence that contains an RNA polymerase binding site, transcription start site, and/or TATA box and assists or promotes the transcription and expression of an associated transcribable polynucleotide sequence and/or gene (or transgene). A promoter can be synthetically produced, varied or derived from a known or naturally occurring promoter sequence or other promoter sequence. A promoter can also include a chimeric promoter comprising a combination of two or more heterologous sequences. A promoter of the present application can thus include variants of promoter sequences that are similar in composition, but not identical to, other promoter sequence(s) known or provided herein. A promoter can be classified according to a variety of criteria relating to the pattern of expression of an associated coding or transcribable sequence or gene (including a transgene) operably linked to the promoter, such as constitutive, developmental, tissue-specific, inducible, etc. Promoters that drive expression in all or most tissues of the plant are referred to as “constitutive” promoters. Promoters that drive expression during certain periods or stages of development are referred to as “developmental” promoters. Promoters that drive enhanced expression in certain tissues of the plant relative to other plant tissues are referred to as “tissue-enhanced” or “tissue-preferred” promoters. Thus, a “tissue-preferred” promoter causes relatively higher or preferential expression in a specific tissue(s) of the plant, but with lower levels of expression in other tissue(s) of the plant. Promoters that express within a specific tissue(s) of the plant, with little or no expression in other plant tissues, are referred to as “tissue-specific” promoters. An “inducible” promoter is a promoter that initiates transcription in response to an environmental stimulus such as cold, drought or light, or other stimuli, such as wounding or chemical application. A promoter can also be classified in terms of its origin, such as being heterologous, homologous, chimeric, synthetic, etc. A “heterologous” promoter is a promoter sequence having a different origin relative to its associated transcribable sequence, coding sequence, or gene (or transgene), and/or not naturally occurring in the plant species to be transformed.

Examples describing a promoter that can be used herein include, without limitation, U.S. Pat. No. 6,437,217 (maize RS81 promoter), U.S. Pat. No. 5,641,876 (rice actin promoter), U.S. Pat. No. 6,426,446 (maize RS324 promoter), U.S. Pat. No. 6,429,362 (maize PR-1 promoter), U.S. Pat. No. 6,232,526 (maize A3 promoter), U.S. Pat. No. 6,177,611 (constitutive maize promoters), U.S. Pat. Nos. 5,322,938, 5,352,605, 5,359,142 and 5,530,196 (35S promoter), U.S. Pat. No. 6,433,252 (maize L3 oleosin promoter), U.S. Pat. No. 6,429,357 (rice actin 2 promoter as well as a rice actin 2 intron), U.S. Pat. No. 5,837,848 (root specific promoter), U.S. Pat. No. 6,294,714 (light inducible promoters), U.S. Pat. No. 6,140,078 (salt inducible promoters), U.S. Pat. No. 6,252,138 (pathogen inducible promoters), U.S. Pat. No. 6,175,060 (phosphorus deficiency inducible promoters), U.S. Pat. No. 6,635,806 (gamma-coixin promoter), and U.S. patent application Ser. No. 09/757,089 (maize chloroplast aldolase promoter). Additional promoters that can find use are a nopaline synthase (NOS) promoter (Ebert et al., 1987), the octopine synthase (OCS) promoter (which is carried on tumor-inducing plasmids of Agrobacterium tumefaciens), the caulimovirus promoters such as the cauliflower mosaic virus (CaMV) 19S promoter (Lawton et al., Plant Molecular Biology (1987) 9: 315-324), the CaMV 35S promoter (Odell et al., Nature (1985) 313: 810-812), the figwort mosaic virus 35S-promoter (U.S. Pat. Nos. 6,051,753; 5,378,619), the sucrose synthase promoter (Yang and Russell, Proceedings of the National Academy of Sciences, USA (1990) 87: 4144-4148), the R gene complex promoter (Chandler et al., Plant Cell (1989) 1: 1175-1183), and the chlorophyll a/b binding protein gene promoter, PC1SV (U.S. Pat. No. 5,850,019), and AGRtu.nos (GenBank Accession V00087; Depicker et al., Journal of Molecular and Applied Genetics (1982) 1: 561-573; Bevan et al., 1983) promoters.

Promoter hybrids can also be used and usually constructed to enhance transcriptional activity (See U.S. Pat. No. 5,106,739), or to combine desired transcriptional activity, inducibility and tissue specificity or developmental specificity. Promoters that function in plants include but are not limited to promoters that are inducible, viral, synthetic, constitutive, temporally regulated, spatially regulated, and spatio-temporally regulated. Other promoters that are tissue-enhanced, tissue-specific, or developmentally regulated are also known in the art and envisioned to have utility in the practice of this disclosure.

As used herein, the term “heterologous” in reference to a promoter is a promoter sequence having a different origin relative to its associated transcribable DNA sequence, coding sequence or gene (or transgene), and/or not naturally occurring in the plant species to be transformed. In addition, the term “heterologous” can refer more broadly to a combination of two or more DNA molecules or sequences, such as a promoter and an associated transcribable DNA sequence, coding sequence or gene, when such a combination is man-made and not normally found in nature.

The term “recombinant” in reference to a polynucleotide (DNA or RNA) molecule, protein, construct, vector, etc., refers to a polynucleotide or protein molecule or sequence that is man-made and not normally found in nature, and/or is present in a context in which it is not normally found in nature, including a polynucleotide (DNA or RNA) molecule, protein, construct, etc., comprising a combination of polynucleotide or protein sequences that would not naturally occur contiguously or in close proximity together without human intervention, and/or a polynucleotide molecule, protein, construct, etc., comprising at least two polynucleotide or protein sequences that are heterologous with respect to each other. A recombinant polynucleotide or protein molecule, construct, etc., can comprise polynucleotide or protein sequence(s) that is/are (i) separated from other polynucleotide or protein sequence(s) that exist in proximity to each other in nature, and/or (ii) adjacent to (or contiguous with) other polynucleotide or protein sequence(s) that are not naturally in proximity with each other. Such a recombinant polynucleotide molecule, protein, construct, etc., can also refer to a polynucleotide or protein molecule or sequence that has been genetically engineered and/or constructed outside of a cell. For example, a recombinant DNA molecule can comprise any suitable plasmid, vector, etc., and can include a linear or circular DNA molecule. Such plasmids, vectors, etc., can contain various maintenance elements including a prokaryotic origin of replication and selectable marker, as well as one or more transgenes or expression cassettes perhaps in addition to a plant selectable marker gene, etc.

In one aspect, methods and compositions provided herein comprise a vector. As used herein, the terms “vector” or “plasmid” are used interchangeably and refer to a circular, double-stranded DNA molecule that is physically separate from chromosomal DNA. In one aspect, a plasmid or vector used herein is capable of replication in vivo. A “transformation vector,” as used herein, is a plasmid that is capable of transforming a plant cell. In an aspect, a plasmid provided herein is a bacterial plasmid. In another aspect, a plasmid provided herein is an Agrobacterium Ti plasmid or derived from an Agrobacterium Ti plasmid.

In one aspect, a plasmid or vector provided herein is a recombinant vector. As used herein, the term “recombinant vector” refers to a vector formed by laboratory methods of genetic recombination, such as molecular cloning. In another aspect, a plasmid provided herein is a synthetic plasmid. As used herein, a “synthetic plasmid” is an artificially created plasmid that is capable of the same functions (e.g., replication) as a natural plasmid (e.g., Ti plasmid). Without being limited, one skilled in the art can create a synthetic plasmid de novo via synthesizing a plasmid by individual nucleotides, or by splicing together nucleic acid molecules from different pre-existing plasmids.

As used herein, “modified”, in the context of plants, seeds, plant components, plant cells, and plant genomes, refers to a state containing changes or variations from their natural or native state. For instance, a “native transcript” of a gene refers to an RNA transcript that is generated from an unmodified gene. Typically, a native transcript is a sense transcript. Modified plants or seeds contain molecular changes in their genetic materials, including either genetic or epigenetic modifications. Typically, modified plants or seeds, or a parental or progenitor line thereof, have been subjected to mutagenesis, genome editing (e.g., without being limiting, via methods using site-specific nucleases), genetic transformation (e.g., without being limiting, via methods of Agrobacterium transformation or microprojectile bombardment), or a combination thereof. In one aspect, a modified plant provided herein comprises no non-plant genetic material or sequences. In yet another aspect, a modified plant provided herein comprises no interspecies genetic material or sequences. In one aspect, this disclosure provides methods and compositions related to modified plants, seeds, plant components, plant cells, and products made from modified plants, seeds, plant parts, and plant cells. In one aspect, a modified seed provided herein gives rise to a modified plant provided herein. In one aspect, a modified plant, seed, plant component, plant cell, or plant genome provided herein comprises a recombinant DNA construct or vector provided herein. In another aspect, a product provided herein comprises modified a plant, plant component, plant cell, or plant chromosome or genome provided herein. The present disclosure provides modified plants with desirable or enhanced properties, e.g., without being limiting, disease, insect, or pest tolerance (for example, virus tolerance, bacteria tolerance, fungus tolerance, nematode tolerance, arthropod tolerance, gastropod tolerance); herbicide tolerance; environmental stress resistance; quality improvements such as yield, nutritional enhancements, environmental or stress tolerances; any desirable changes in plant physiology, growth, development, morphology or plant product(s) including starch production, modified oils production, high oil production, modified fatty acid content, high protein production, fruit ripening, enhanced animal and human nutrition, biopolymer production, pharmaceutical peptides and secretable peptides production; improved processing traits; improved digestibility; low raffinose; industrial enzyme production; improved flavor; nitrogen fixation; hybrid seed production; and fiber production.

As used herein, “genome editing” or “editing” refers to targeted mutagenesis, insertion, deletion, inversion, substitution, or translocation of a nucleotide sequence of interest in a genome using a targeted editing technique. A nucleotide sequence of interest can be of any length, e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 1000, at least 2500, at least 5000, at least 10,000, or at least 25,000 nucleotides. A nucleotide sequence of interest can be an endogenous genomic sequence or a transgenic sequence.

As used herein, a “targeted editing technique” refers to any method, protocol, or technique that allows the precise and/or targeted editing of a specific location in a genome (e.g., the editing is not random). Without being limiting, use of a site-specific nuclease is one example of a targeted editing technique. In one aspect, a targeted editing technique is used to edit an endogenous locus or an endogenous gene. In another aspect, a targeted editing technique is used to edit a transgene.

As used herein, “genome engineering” refers to the manupination or synthetic assembly of complete chromosomal DNA that is essentially derived from natural genomic sequences.

As used herein, a “locus” refers to a specific position on a chromosome. Without being limiting, a locus can comprise a polynucleotide that encodes a protein or an RNA. A locus can also comprise a non-coding RNA. A locus can comprise a gene. A locus can comprise a promoter, a 5′-untranslated region (UTR), an exon, an intron, a 3′-UTR, or any combination thereof. A locus can comprise a coding region.

One aspect of the present application relate to methods of screening and selecting cells for targeted edits or desired chromosome recombination via nucleic acid assays. Nucleic acids can be isolated using various techniques. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

The screening and selection of modified, engineered, or transgenic plants or plant cells can be through any methodologies known in the art. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina, PacBio, Ion Torrent, 454) enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known.

Genome editing or targeted editing can be effected via the use of one or more site-specific nucleases. Site-specific nucleases can induce a double-stranded break (DSB) at a target site of a genome sequence that is then repaired by the natural processes of either homologous recombination (HR) or non-homologous end-joining (NHEJ). Sequence modifications, such as insertions, deletions, can occur at the DSB locations via NHEJ repair. If two DSBs flanking one target region are created, the breaks can be repaired via NHEJ by reversing the orientation of the targeted DNA (also referred to as an “inversion”). HR can be used to integrate a donor nucleic acid sequence into a target site. Without being limited by any theory, in order to integrate a donor nucleic acid sequence (or donor molecule) into a DSB, the donor molecule comprises a polynucleotide of interest flanked by a first and second homologous region, where the first and second homologous regions are homologous to each side of the DSB at the target site. Homologous recombination machinery in the cell then repairs the DSB by integrating the donor molecule into the target site.

In one aspect, a genome editing system or method provided here comprises the use of a vector or construct encoding at least 1, at least 2, at least 3, at least 4, at least 5, at least 6, at least 7, at least 8, at least 9, or at least 10 site-specific nuclease. In another aspect, a cell provided herein already comprises a site-specific nuclease. In an aspect, a polynucleotide encoding a site-specific nuclease provided herein is stably transformed into a cell. In another aspect, a polynucleotide encoding a site-specific nuclease provided herein is transiently transformed into a cell. In another aspect, a polynucleotide encoding a site-specific nuclease is under the control of a regulatable promoter, a constitutive promoter, a tissue specific promoter, or any promoter useful for expression of the site-specific nuclease.

In one aspect, a vector comprises in cis a cassette encoding a site-specific nuclease and a donor molecule such that when contacted with the genome of a cell, the site-specific nuclease enables site-specific integration of the donor molecule. In one aspect, a first vector comprises a cassette encoding a site-specific nuclease and a second vector comprises a donor molecule such that when contacted with the genome of a cell, the site-specific nuclease provided in trans enables site-specific integration of the donor molecule.

Site-specific nucleases provided herein can be used as part of a targeted editing technique for chromosome engineering. Non-limiting examples of site-specific nucleases used in methods and/or compositions provided herein include meganucleases, zinc finger nucleases (ZFNs), transcription activator-like effector nucleases (TALENs), RNA-guided nucleases (e.g., Cas9 and Cpf1), a recombinase (without being limiting, for example, a serine recombinase attached to a DNA recognition motif, a tyrosine recombinase attached to a DNA recognition motif), a transposase (without being limiting, for example, a DNA transposase attached to a DNA binding domain), or any combination thereof. In one aspect, a method provided herein comprises the use of one or more, two or more, three or more, four or more, or five or more site-specific nucleases to induce one, two, three, four, five, or more than five DSBs at one, two, three, four, five, or more than five target sites.

In one aspect, a genome editing system provided herein (e.g., a meganuclease, a ZFN, a TALEN, a CRISPR/Cas9 system, a CRISPR/Cpf1 system, a recombinase, a transposase), or a combination of genome editing systems provided herein, is used in a method to introduce one or more insertions, deletions, substitutions, or inversions to a locus or chromosome recombination and/or rearrangement in a cell

Site-specific nucleases, such as meganucleases, ZFNs, TALENs, Argonaute proteins (non-limiting examples of Argonaute proteins include Thermus thermophilus Argonaute (TtAgo), Pyrococcus furiosus Argonaute (PfAgo), Natronobacterium gregoryi Argonaute (NgAgo), homologs thereof, or modified versions thereof), Cas9 nucleases (non-limiting examples of RNA-guided nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1 (also known as Cas12a), homologs thereof, or modified versions thereof), induce a double-strand DNA break at the target site of a genomic sequence that is then repaired by the natural processes of HR or NHEJ. Sequence modifications then occur at the cleaved sites, which can include inversions, deletions, or insertions that result in gene disruption in the case of NHEJ, or integration of nucleic acid sequences by HR.

In an aspect, a site-specific nuclease provided herein is selected from the group consisting of a zinc-finger nuclease, a meganuclease, an RNA-guided nuclease, a TALE-nuclease, a recombinase, a transposase, or any combination thereof. In another aspect, a site-specific nuclease provided herein is selected from the group consisting of a Cas9 or a Cpf1. In another aspect a site-specific nuclease provided herein is selected from the group consisting of a Cas1, a Cas1B, a Cas2, a Cas3, a Cas4, a Cas5, a Cas6, a Cas7, a Cas8, a Cas9, a Cas10, a Csy1, a Csy2, a Csy3, a Cse1, a Cse2, a Csc1, a Csc2, a Csa5, a Csn2, a Csm2, a Csm3, a Csm4, a Csm5, a Csm6, a Cmr1, a Cmr3, a Cmr4, a Cmr5, a Cmr6, a Csb1, a Csb2, a Csb3, a Csx17, a Csx14, a Csx10, a Csx16, a CsaX, a Csx3, a Csx1, a Csx15, a Csf1, a Csf2, a Csf3, a Csf4, a Cpf1 (also known as Cas12a), a homolog thereof, or a modified version thereof.

In one aspect, a genome editing system described here can comprise a site-directed nuclease having a recombinase domain or a modification thereof. In an aspect, a tyrosine recombinase attached to a DNA recognition motif provided herein is selected from the group consisting of a Cre recombinase, a Gin recombinase a Flp recombinase, and a Tnp1 recombinase. In an aspect, a Cre recombinase or a Gin recombinase provided herein is tethered to a zinc-finger DNA binding domain. The Flp-FRT site-directed recombination system comes from the 2μ plasmid from the baker's yeast Saccharomyces cerevisiae. In this system, Flp recombinase (flippase) recombines sequences between flippase recognition target (FRT) sites. FRT sites comprise 34 nucleotides. Flp binds to the “arms” of the FRT sites (one arm is in reverse orientation) and cleaves the FRT site at either end of an intervening nucleic acid sequence. After cleavage, Flp recombines nucleic acid sequences between two FRT sites. Cre-lox is a site-directed recombination system derived from the bacteriophage P1 that is similar to the Flp-FRT recombination system. Cre-lox can be used to invert a nucleic acid sequence, delete a nucleic acid sequence, or translocate a nucleic acid sequence. In this system, Cre recombinase recombines a pair of lox nucleic acid sequences. Lox sites comprise 34 nucleotides, with the first and last 13 nucleotides (arms) being palindromic. During recombination, Cre recombinase protein binds to two lox sites on different nucleic acids and cleaves at the lox sites. The cleaved nucleic acids are spliced together (reciprocally translocated) and recombination is complete. In another aspect, a lox site provided herein is a loxP, lox 2272, loxN, lox 511, lox 5171, lox71, lox66, M2, M3, M7, or M11 site.

In another aspect, a serine recombinase attached to a DNA recognition motif provided herein is selected from the group consisting of a PhiC31 integrase, an R4 integrase, and a TP-901 integrase. In another aspect, a DNA transposase attached to a DNA binding domain provided herein is selected from the group consisting of a TALE-piggyBac and TALE-Mutator.

### ZFNs

In one aspect, a genome editing system described here can comprise a ZFN or a modification thereof. ZFNs are synthetic proteins consisting of an engineered zinc finger DNA-binding domain fused to the cleavage domain of the FokI restriction nuclease. ZFNs can be designed to cleave almost any long stretch of double-stranded DNA for modification of the zinc finger DNA-binding domain. ZFNs form dimers from monomers composed of a non-specific DNA cleavage domain of FokI nuclease fused to a zinc finger array engineered to bind a target DNA sequence.

The DNA-binding domain of a ZFN is typically composed of 3-4 zinc-finger arrays. The amino acids at positions −1, +2, +3, and +6 relative to the start of the zinc finger ∞-helix, which contribute to site-specific binding to the target DNA, can be changed and customized to fit specific target sequences. The other amino acids form the consensus backbone to generate ZFNs with different sequence specificities. Rules for selecting target sequences for ZFNs are known in the art.

The FokI nuclease domain requires dimerization to cleave DNA and therefore two ZFNs with their C-terminal regions are needed to bind opposite DNA strands of the cleavage site (separated by 5-7 nt). The ZFN monomer can cute the target site if the two-ZF-binding sites are palindromic. The term ZFN, as used herein, is broad and includes a monomeric ZFN that can cleave double stranded DNA without assistance from another ZFN. The term ZFN is also used to refer to one or both members of a pair of ZFNs that are engineered to work together to cleave DNA at the same site.

Without being limited by any scientific theory, because the DNA-binding specificities of zinc finger domains can in principle be re-engineered using one of various methods, customized ZFNs can theoretically be constructed to target nearly any gene sequence. Publicly available methods for engineering zinc finger domains include Context-dependent Assembly (CoDA), Oligomerized Pool Engineering (OPEN), and Modular Assembly.

### Meganucleases

In one aspect, a genome editing system described here can comprise a meganuclease or a modification thereof. Meganucleases, which are commonly identified in microbes, are unique enzymes with high activity and long recognition sequences (>14 nt) resulting in site-specific digestion of target DNA. Engineered versions of naturally occurring meganucleases typically have extended DNA recognition sequences (for example, 14 to 40 nt). The engineering of meganucleases can be more challenging than that of ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity.

In one aspect, a method and/or composition provided herein comprises one or more, two or more, three or more, four or more, or five or more meganucleases. In another aspect, a meganuclease provided herein is capable of generating a targeted DSB. In one aspect, vectors comprising polynucleotides encoding one or more, two or more, three or more, four or more, or five or more meganucleases are provided to a cell by transformation methods known in the art (e.g., without being limiting, viral transfection, particle bombardment, PEG-mediated protoplast transfection or Agrobacterium-mediated transformation).

### TALENs

In one aspect, a genome editing system described here can comprise a TALEN-based nuclease or a modification thereof. TALENs are artificial restriction enzymes generated by fusing the transcription activator-like effector (TALE) DNA binding domain to a FokI nuclease domain. When each member of a TALEN pair binds to the DNA sites flanking a target site, the FokI monomers dimerize and cause a double-stranded DNA break at the target site. Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity.

TALENs are artificial restriction enzymes generated by fusing the transcription activator-like effector (TALE) DNA binding domain to a nuclease domain. In one aspect, the nuclease is selected from a group consisting of PvuII, MutH, TevI and FokI, AZwI, MlyI, SdaI, StsI, CleDORF, Clo051, Pept071. When each member of a TALEN pair binds to the DNA sites flanking a target site, the FokI monomers dimerize and cause a double-stranded DNA break at the target site.

The term TALEN, as used herein, is broad and includes a monomeric TALEN that can cleave double stranded DNA without assistance from another TALEN. The term TALEN is also used to refer to one or both members of a pair of TALENs that work together to cleave DNA at the same site.

Transcription activator-like effectors (TALEs) can be engineered to bind practically any DNA sequence. TALE proteins are DNA-binding domains derived from various plant bacterial pathogens of the genus Xanthomonas. The X pathogens secrete TALEs into the host plant cell during infection. The TALE moves to the nucleus, where it recognizes and binds to a specific DNA sequence in the promoter region of a specific DNA sequence in the promoter region of a specific gene in the host genome. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. PvuII, MutH, and TevI cleavage domains are useful alternatives to FokI and FokI variants for use with TALEs. PvuII functions as a highly specific cleavage domain when coupled to a TALE (See Yank et al. 2013. PLoS One. 8: e82539). MutH is capable of introducing strand-specific nicks in DNA (See Gabsalilow et al. 2013. Nucleic Acids Research. 41: e83). TevI introduces double-stranded breaks in DNA at targeted sites (See Beurdeley et al., 2013. Nature Communications. 4: 1762).

The relationship between amino acid sequence and DNA recognition of the TALE binding domain allows for designable proteins. Software programs such as DNA Works can be used to design TALE constructs. Other methods of designing TALE constructs are known to those of skill in the art. See Doyle et al., Nucleic Acids Research (2012) 40: W117-122; Cermak et al., Nucleic Acids Research (2011). 39:e82; and tale-nt.cac.cornell.edu/about.

In one aspect, a method and/or composition provided herein comprises one or more, two or more, three or more, four or more, or five or more TALENs. In another aspect, a TALEN provided herein is capable of generating a targeted DSB. In one aspect, vectors comprising polynucleotides encoding one or more, two or more, three or more, four or more, or five or more TALENs are provided to a cell by transformation methods known in the art (e.g., without being limiting, viral transfection, particle bombardment, PEG-mediated protoplast transfection or Agrobacterium-mediated transformation).

### RNA-Guided Nucleases

In one aspect, a genome editing system described here can comprise a RNA-guided nuclease, e.g., a CRISPR/Cas9 nuclease or a CRISPR/Cpf1 nuclease, or a modification thereof. A CRISPR/Cas9 system or a CRISPR/Cpf1 system are alternatives to the FokI-based methods ZFN and TALEN. The CRISPR systems are based on RNA-guided engineered nucleases that use complementary base pairing to recognize DNA sequences at target sites.

While not being limited by any particular scientific theory, CRISPR/Cas nucleases are part of the adaptive immune system of bacteria and archaea, protecting them against invading nucleic acids such as viruses by cleaving target DNA in a sequence-dependent manner. The immunity is acquired by the integration of short fragments of the invading DNA, known as spacers, between ˜20 nucleotide long CRISPR repeats at the proximal end of a CRISPR locus (a CRISPR array). A well described Cas protein is the Cas9 nuclease (also known as Csn1), which is part of the Class 2, type II CRISPR/Cas system in Streptococcus pyogenes. See Makarova et al. Nature Reviews Microbiology (2015) doi: 10.1038/nrmicro3569. Cas9 comprises an RuvC-like nuclease domain at its amino terminus and an HNH-like nuclease domain positioned in the middle of the protein. Cas9 proteins also contain a PAM-interacting (PI) domain, a recognition lobe (REC), and a BH domain. The Cpf1 nuclease, another type II system, acts in a similar manner to Cas9, but Cpf1 does not require a tracrRNA. See Cong et al. Science (2013) 339: 819-823; Zetsche et al., Cell (2015) doi: 10.1016/j.cell.2015.09.038; U.S. Patent Publication No. 2014/0068797; U.S. Patent Publication No. 2014/0273235; U.S. Patent Publication No. 2015/0067922; U.S. Pat. Nos. 8,697,359; 8,771,945; 8,795,965; 8,865,406; 8,871,445; 8,889,356; 8,889,418; 8,895,308; and 8,906,616, each of which is herein incorporated by reference in its entirety.

When Cas9 or Cpf1 cleaves targeted DNA, endogenous double stranded break (DSB) repair mechanisms are activated. DSBs can be repaired via non-homologous end joining, which can incorporate insertions or deletions (indels) into the targeted locus. If two DSBs flanking one target region are created, the breaks can be repaired by reversing the orientation of the targeted DNA. Alternatively, if a donor polynucleotide with homology to the target DNA sequence is provided, the DSB can be repaired via homology-directed repair. This repair mechanism allows for the precise integration of a donor polynucleotide into the targeted DNA sequence.

While not being limited by any particular scientific theory, in Class 2, type II CRISPR/Cas systems, CRISPR arrays, including spacers, are transcribed during encounters with recognized invasive DNA and are processed into small interfering CRISPR RNAs (crRNAs), which are approximately 40 nucleotides in length. The crRNAs hybridize with trans-activating crRNAs (tracrRNAs) to activate and guide the Cas9 nuclease to a target site. Nucleic acid molecules provided herein can combine a crRNA and a tracrRNA into one nucleic acid molecule in what is herein referred to as a “single-chain guide RNA (sgRNA).” A prerequisite for cleavage of the target site is the presence of a conserved protospacer-adjacent motif (PAM) downstream of the target DNA, which usually has the sequence 5-NGG-3 but less frequently NAG. Specificity is provided by the so-called “Seed sequence” approximately 12 bases upstream of the PAM, which must match between the RNA and target DNA. Cpf1 acts in a similar manner to Cas9, but Cpf1 does not require a tracrRNA. Therefore, in an aspect utilizing Cpf1 a sgRNA can be replaced by a crRNA. In an aspect, when two or more sgRNAs are provided herein, the first sgRNA and the second sgRNA are complementary to different strands of a double-stranded DNA molecule. In another aspect, when two or more sgRNAs are provided herein, the first sgRNA and the second sgRNA are complementary to the same strand of a double-stranded DNA molecule.

In one aspect, a method and/or composition provided herein comprises one or more, two or more, three or more, four or more, or five or more Cas9 nucleases. In one aspect, a method and/or composition provided herein comprises one or more polynucleotides encoding one or more, two or more, three or more, four or more, or five or more Cas9 nucleases. In another aspect, a Cas9 nuclease provided herein is capable of generating a targeted DSB. In one aspect, a method and/or composition provided herein comprises one or more, two or more, three or more, four or more, or five or more Cpf1 nucleases. In one aspect, a method and/or composition provided herein comprises one or more polynucleotides encoding one or more, two or more, three or more, four or more, or five or more Cpf1 nucleases. In another aspect, a Cpf1 nuclease provided herein is capable of generating a targeted DSB.

When a Cas9 nuclease hybridizes to a target site via an sgRNA, Cas9 produces two blunt-end cuts in the double-stranded DNA. The “target strand” of the double-stranded DNA is complementary to the sgRNA, while the “non-target strand” comprises the PAM motif adjacent to, and on the 3′ end of, the cut site on the non-target strand. Cas9 holds the target stand and the PAM motif, but the 3′ cut end of the non-target strand is free and is referred to as the “3′ flap.” In one aspect, the 3′ flap comprises at least 10, at least 15, at least 20, at least 25, at least 26, at least 27, at least 28, at least 29, at least 30, at least 31, at least 32, at least 33, at least 34, at least 35, at least 36, at least 37, at least 38, at least 39, or at least 40 nucleotides.

In one aspect, vectors comprising polynucleotides encoding a site-specific nuclease, and optionally one or more, two or more, three or more, or four or more sgRNAs are provided to a plant cell by transformation methods known in the art (e.g., without being limiting, particle bombardment, PEG-mediated protoplast transfection or Agrobacterium-mediated transformation). In one aspect, vectors comprising polynucleotides encoding a Cas9 nuclease, and optionally one or more, two or more, three or more, or four or more sgRNAs are provided to a plant cell by transformation methods known in the art (e.g., without being limiting, particle bombardment, PEG-mediated protoplast transfection or Agrobacterium-mediated transformation). In another aspect, vectors comprising polynucleotides encoding a Cpf1 and, optionally one or more, two or more, three or more, or four or more crRNAs are provided to a cell by transformation methods known in the art (e.g., without being limiting, viral transfection, particle bombardment, PEG-mediated protoplast transfection or Agrobacterium-mediated transformation).

### Targeted Cell Types

In one aspect, methods and compositions provided herein can be used to edit a locus in a eukaryotic cell. In one aspect, a eukaryotic cell provided herein is part of a multicellular eukaryotic organism. In another aspect, a eukaryotic cell provided herein is a unicellular organism. In another aspect, a eukaryotic cell provided herein is selected from the group consisting of an animal cell, a plant cell, a fungus cell, and a protozoan cell. In one aspect, an animal cell provided herein is selected from the group consisting of an insect cell, an arachnid cell, an arthropod cell, a crustacean cell, a rotifer cell, a cnidarian cell, a Platyhelminthes cell, a mollusk cell, a gastropod cell, a nematode cell, an annelid cell, a vertebrate cell, a mammal cell, an avian cell, a fish cell, a reptile cell, and an amphibian cell. In another aspect a plant cell provided herein is a monocot cell or a dicot cell. In still another aspect a plant cell provided herein is an algae cell. In yet another aspect, a plant cell provided herein is selected from the group consisting of a corn cell, a wheat cell, a sorghum cell, a canola cell, a soybean cell, an alfalfa cell, a cotton cell, and a rice cell. In still another aspect, a plant cell provided herein is selected from the group consisting of an Acacia cell, an alfalfa cell, an aneth cell, an apple cell, an apricot cell, an artichoke cell, an arugula cell, an asparagus cell, an avocado cell, a banana cell, a barley cell, a bean cell, a beet cell, a blackberry cell, a blueberry cell, a broccoli cell, a Brussels sprout cell, a cabbage cell, a canola cell, a cantaloupe cell, a carrot cell, a cassava cell, a cauliflower cell, a celery cell, a Chinese cabbage cell, a cherry cell, a cilantro cell, a citrus cell, a clementine cell, a coffee cell, a corn cell, a cotton cell, a cucumber cell, a Douglas fir cell, an eggplant cell, an endive cell, an escarole cell, an eucalyptus cell, a fennel cell, a fig cell, a forest tree cell, a gourd cell, a grape cell, a grapefruit cell, a honey dew cell, a jicama cell, kiwifruit cell, a lettuce cell, a leek cell, a lemon cell, a lime cell, a Loblolly pine cell, a mango cell, a maple tree cell, a melon cell, a mushroom cell, a nectarine cell, a nut cell, an oat cell, an okra cell, an onion cell, an orange cell, an ornamental plant cell, a papaya cell, a parsley cell, a pea cell, a peach cell, a peanut cell, a pear cell, a pepper cell, a persimmon cell, a pine cell, a pineapple cell, a plantain cell, a plum cell, a pomegranate cell, a poplar cell, a potato cell, a pumpkin cell, a quince cell, a radiata pine cell, a radicchio cell, a radish cell, a rapeSeed cell, a raspberry cell, a rice cell, a rye cell, a sorghum cell, a Southern pine cell, a soybean cell, a spinach cell, a squash cell, a strawberry cell, a sugar beet cell, a sugarcane cell, a sunflower cell, a sweet corn cell, a sweet potato cell, a sweetgum cell, a tangerine cell, a tea cell, a tobacco cell, a tomato cell, a turf cell, a vine cell, watermelon cell, a wheat cell, a yam cell, and a zucchini cell. In another aspect, a plant cell provided herein is selected from the group consisting of a corn cell, a soybean cell, a canola cell, a cotton cell, a wheat cell, and a sugarcane cell.

In still another aspect, an engineered plant provided herein is an alga. In yet another aspect, an engineered plant or seed provided herein is selected from the group consisting of a corn plant, a wheat plant, a sorghum plant, a canola plant, a soybean plant, an alfalfa plant, a cotton plant, and a rice plant. In still another aspect, an engineered plant or seed provided herein is selected from the group consisting of an Acacia plant, an alfalfa plant, an aneth plant, an apple plant, an apricot plant, an artichoke plant, an arugula plant, an asparagus plant, an avocado plant, a banana plant, a barley plant, a bean plant, a beet plant, a blackberry plant, a blueberry plant, a broccoli plant, a Brussels sprout plant, a cabbage plant, a canola plant, a cantaloupe plant, a carrot plant, a cassava plant, a cauliflower plant, a celery plant, a Chinese cabbage plant, a cherry plant, a cilantro plant, a citrus plant, a clementine plant, a coffee plant, a corn plant, a cotton plant, a cucumber plant, a Douglas fir plant, an eggplant plant, an endive plant, an escarole plant, an eucalyptus plant, a fennel plant, a fig plant, a forest tree plant, a gourd plant, a grape plant, a grapefruit plant, a honey dew plant, a jicama plant, kiwifruit plant, a lettuce plant, a leek plant, a lemon plant, a lime plant, a Loblolly pine plant, a mango plant, a maple tree plant, a melon plant, a mushroom plant, a nectarine plant, a nut plant, an oat plant, an okra plant, an onion plant, an orange plant, an ornamental plant, a papaya plant, a parsley plant, a pea plant, a peach plant, a peanut plant, a pear plant, a pepper plant, a persimmon plant, a pine plant, a pineapple plant, a plantain plant, a plum plant, a pomegranate plant, a poplar plant, a potato plant, a pumpkin plant, a quince plant, a radiata pine plant, a radicchio plant, a radish plant, a rapeSeed plant, a raspberry plant, a rice plant, a rye plant, a sorghum plant, a Southern pine plant, a soybean plant, a spinach plant, a squash plant, a strawberry plant, a sugar beet plant, a sugarcane plant, a sunflower plant, a sweet corn plant, a sweet potato plant, a sweetgum plant, a tangerine plant, a tea plant, a tobacco plant, a tomato plant, a turf plant, a vine plant, watermelon plant, a wheat plant, a yam plant, and a zucchini plant. In another aspect, a plant provided herein is selected from the group consisting of a corn plant, a soybean plant, a canola plant, a cotton plant, a wheat plant, and a sugarcane plant.

In still another aspect, a modified chromosome provided herein is from an alga. In yet another aspect, a modified chromosome provided herein is selected from the group consisting of a corn chromosome, a wheat chromosome, a sorghum chromosome, a canola chromosome, a soybean chromosome, an alfalfa chromosome, a cotton chromosome, and a rice chromosome. In still another aspect, a modified chromosome provided herein is selected from the group consisting of an Acacia chromosome, an alfalfa chromosome, an aneth chromosome, an apple chromosome, an apricot chromosome, an artichoke chromosome, an arugula chromosome, an asparagus chromosome, an avocado chromosome, a banana chromosome, a barley chromosome, a bean chromosome, a beet chromosome, a blackberry chromosome, a blueberry chromosome, a broccoli chromosome, a Brussels sprout chromosome, a cabbage chromosome, a canola chromosome, a cantaloupe chromosome, a carrot chromosome, a cassava chromosome, a cauliflower chromosome, a celery chromosome, a Chinese cabbage chromosome, a cherry chromosome, a cilantro chromosome, a citrus chromosome, a clementine chromosome, a coffee chromosome, a corn chromosome, a cotton chromosome, a cucumber chromosome, a Douglas fir chromosome, an eggplant chromosome, an endive chromosome, an escarole chromosome, an eucalyptus chromosome, a fennel chromosome, a fig chromosome, a forest tree chromosome, a gourd chromosome, a grape chromosome, a grapefruit chromosome, a honey dew chromosome, a jicama chromosome, kiwifruit chromosome, a lettuce chromosome, a leek chromosome, a lemon chromosome, a lime chromosome, a Loblolly pine chromosome, a mango chromosome, a maple tree chromosome, a melon chromosome, a mushroom chromosome, a nectarine chromosome, a nut chromosome, an oat chromosome, an okra chromosome, an onion chromosome, an orange chromosome, an plant chromosome chromosome, a papaya chromosome, a parsley chromosome, a pea chromosome, a peach chromosome, a peanut chromosome, a pear chromosome, a pepper chromosome, a persimmon chromosome, a pine chromosome, a pineapple chromosome, a plantain chromosome, a plum chromosome, a pomegranate chromosome, a poplar chromosome, a potato chromosome, a pumpkin chromosome, a quince chromosome, a radiata pine chromosome, a radicchio chromosome, a radish chromosome, a rapeSeed chromosome, a raspberry chromosome, a rice chromosome, a rye chromosome, a sorghum chromosome, a Southern pine chromosome, a soybean chromosome, a spinach chromosome, a squash chromosome, a strawberry chromosome, a sugar beet chromosome, a sugarcane chromosome, a sunflower chromosome, a sweet corn chromosome, a sweet potato chromosome, a sweetgum chromosome, a tangerine chromosome, a tea chromosome, a tobacco chromosome, a tomato chromosome, a turf chromosome, a vine chromosome, watermelon chromosome, a wheat chromosome, a yam chromosome, and a zucchini chromosome.

According to one aspect, the present disclosure provides a modified plant cell produced by any one of the methods provided herein. In another aspect, the present disclosure provides a modified chromosome produced by any one of the methods provided herein. In still another aspect, the present disclosure provides a modified cell comprising a modified chromosome provided herein. In still a further aspect, this disclosure provides a modified plant or modified plant tissue regenerated from a modified cell provided herein. In still another aspect, the present disclosure provides a product comprising a modified chromosome provided herein. In an aspect, the present disclosure provides a product comprising a modified cell provided herein. As used herein, a “product” refers to any article or substance that is intended for human use, human consumption, animal use, or animal consumption, including any component, part, or accessory that comprises a modified cell or modified chromosome provided herein.

The methods and compositions provided herein are capable of editing any locus in a genome. Also provided herein are chromosomes edited by using the methods and compositions provided herein. In an aspect, a genome provided herein is a nuclear genome, a mitochondrial genome, or a plastid genome. In another aspect, a plastid genome provided herein comprises a chloroplast genome. In one aspect, a method provided herein generates a double-stranded break on a chromosome. In an aspect, a chromosome provided herein is a nuclear chromosome, a mitochondrial chromosome, or a chloroplast chromosome. In another aspect a chromosome provided herein is a supernumerary chromosome or an artificial chromosome. Supernumerary, or B chromosomes, are extra chromosomes found in addition to the normal diploid complement of chromosomes in a cell. Supernumerary chromosomes are dispensable and not required for normal development of a cell or organism.

### Transformation

A method for chromosomal engineering or genome editing disclosed here may involve transient transfection or stable transformation of a cell of interest (e.g., a plant cell). According to one aspect of the present application, methods are provided for transforming a cell, tissue or explant with a recombinant DNA molecule or construct comprising a transcribable DNA sequence or transgene operably linked to a promoter to produce a transgenic or genome edited cell. According to another aspect of the present application, methods are provided for transforming a plant cell, tissue or explant with a recombinant DNA molecule or construct comprising a transcribable DNA sequence or transgene operably linked to a plant-expressible promoter to produce a transgenic or genome edited plant or plant cell. As used herein, a “transgene” refers to a polynucleotide that has been transferred into a genome by any method known in the art.

Numerous methods for transforming chromosomes or plastids in a plant cell with a recombinant DNA molecule or construct are known in the art, which can be used according to methods of the present application to produce a transgenic plant cell and plant. Any suitable method or technique for transformation of a plant cell known in the art can be used according to present methods. Effective methods for transformation of plants include bacterially mediated transformation, such as Agrobacterium-mediated or Rhizobium-mediated transformation and microprojectile bombardment-mediated transformation. A variety of methods are known in the art for transforming explants with a transformation vector via bacterially mediated transformation or microprojectile bombardment and then subsequently culturing, etc., those explants to regenerate or develop transgenic plants. Other methods for plant transformation, such as microinjection, electroporation, vacuum infiltration, pressure, sonication, silicon carbide fiber agitation, PEG-mediated transformation, etc., are also known in the art. Transgenic plants produced by these transformation methods can be chimeric or non-chimeric for the transformation event depending on the methods and explants used.

Methods of transforming plant cells are well known by persons of ordinary skill in the art. For instance, specific instructions for transforming plant cells by microprojectile bombardment with particles coated with recombinant DNA are found in U.S. Pat. Nos. 5,550,318; 5,538,880 6,160,208; 6,399,861; and 6,153,812 and Agrobacterium-mediated transformation is described in U.S. Pat. Nos. 5,159,135; 5,824,877; 5,591,616; 6,384,301; 5,750,871; 5,463,174; and 5,188,958, all of which are incorporated herein by reference. Additional methods for transforming plants can be found in, for example, Compendium of Transgenic Crop Plants (2009) Blackwell Publishing. Any appropriate method known to those skilled in the art can be used to transform a plant cell with any of the nucleic acid molecules provided herein.

Recipient cell or explant targets for transformation include, but are not limited to, a seed cell, a fruit cell, a leaf cell, a cotyledon cell, a hypocotyl cell, a meristem cell, an embryo cell, an endosperm cell, a root cell, a shoot cell, a stem cell, a pod cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, a phloem cell, a bud cell, or a vascular tissue cell. In another aspect, this disclosure provides a plant chloroplast. In a further aspect, this disclosure provides an epidermal cell, a stomata cell, a trichome cell, a root hair cell, a storage root cell, or a tuber cell. In another aspect, this disclosure provides a protoplast. In another aspect, this disclosure provides a plant callus cell. Any cell from which a fertile plant can be regenerated is contemplated as a useful recipient cell for practice of this disclosure. Callus can be initiated from various tissue sources, including, but not limited to, immature embryos or parts of embryos, seedling apical meristems, microspores, and the like. Those cells which are capable of proliferating as callus can serve as recipient cells for transformation. Practical transformation methods and materials for making transgenic plants of this disclosure (e.g., various media and recipient target cells, transformation of immature embryos, and subsequent regeneration of fertile transgenic plants) are disclosed, for example, in U.S. Pat. Nos. 6,194,636 and 6,232,526 and U.S. Patent Application Publication 2004/0216189, all of which are incorporated herein by reference. Transformed explants, cells or tissues can be subjected to additional culturing steps, such as callus induction, selection, regeneration, etc., as known in the art. Transformed cells, tissues or explants containing a recombinant DNA insertion can be grown, developed or regenerated into transgenic plants in culture, plugs or soil according to methods known in the art. In one aspect, this disclosure provides plant cells that are not reproductive material and do not mediate the natural reproduction of the plant. In another aspect, this disclosure also provides plant cells that are reproductive material and mediate the natural reproduction of the plant. In another aspect, this disclosure provides plant cells that cannot maintain themselves via photosynthesis. In another aspect, this disclosure provides somatic plant cells. Somatic cells, contrary to germline cells, do not mediate plant reproduction. In one aspect, this disclosure provides a non-reproductive plant cell.

Modified plants can be further crossed to themselves or other plants to produce modified seeds and progeny. A modified plant can also be prepared by crossing a first plant comprising a recombinant DNA sequence insertion with a second plant lacking the insertion. For example, a recombinant DNA sequence can be introduced into a first plant line that is amenable to transformation, which can then be crossed with a second plant line to introgress the recombinant DNA sequence into the second plant line. A modified plant can also be prepared by crossing a modified plant with an unmodified plant. Progeny of these crosses can be further back crossed into the more desirable line multiple times, such as through 6 to 8 generations or back crosses, to produce a progeny plant with substantially the same genotype as the original parental line but for the introduction of the recombinant DNA construct or modified sequence.

A modified plant, cell, or explant provided herein can be of an elite variety or an elite line. An elite variety or an elite line refers to any variety that has resulted from breeding and selection for superior agronomic performance. A modified plant, cell, or explant provided herein can be a hybrid plant, cell, or explant. As used herein, a “hybrid” is created by crossing two plants from different varieties, lines, or species, such that the progeny comprises genetic material from each parent. Skilled artisans recognize that higher order hybrids can be generated as well. For example, a first hybrid can be made by crossing Variety C with Variety D to create a C×D hybrid, and a second hybrid can be made by crossing Variety E with Variety F to create an E×F hybrid. The first and second hybrids can be further crossed to create the higher order hybrid (C×D)×(E×F) comprising genetic information from all four parent varieties. A modified plant provided herein is fertile. A modified plant provided herein is a male or female sterile modified plant, which cannot reproduce without human intervention. In one aspect, a modified plant provided herein reproduces via asexual or vegetative reproduction. In still another aspect, a modified plant provided herein reproduces via sexual reproduction.

A plant selectable marker transgene in a transformation vector or construct of the present application can be used to assist in the selection of transformed cells or tissue due to the presence of a selection agent, such as an antibiotic or herbicide, where the plant selectable marker transgene provides tolerance or resistance to the selection agent. Thus, the selection agent can bias or favor the survival, development, growth, proliferation, etc., of transformed cells expressing the plant selectable marker gene, such as to increase the proportion of transformed cells or tissues in the Ro plant. Commonly used plant selectable marker genes include, for example, those conferring tolerance or resistance to antibiotics, such as kanamycin and paromomycin (nptII), hygromycin B (aph IV), streptomycin or spectinomycin (aadA) and gentamycin (aac3 and aacC4), or those conferring tolerance or resistance to herbicides such as glufosinate (bar or pat), dicamba (DMO) and glyphosate (aroA or Cp4-EPSPS). Plant screenable marker genes can also be used, which provide an ability to visually screen for transformants, such as luciferase or green fluorescent protein (GFP), or a gene expressing a beta glucuronidase or uidA gene (GUS) for which various chromogenic substrates are known. In one aspect, a vector or polynucleotide provided herein comprises at least one marker gene selected from the group consisting of nptII, aph IV, aadA, aac3, aacC4, bar, pat, DMO, EPSPS, aroA, GFP, and GUS.

According to an aspect of the present application, methods for transforming a plant cell, tissue or explant with a recombinant DNA molecule or construct can further include site-directed or targeted integration using site-specific nucleases. According to these methods, a portion of a recombinant DNA donor molecule (e.g., an insertion sequence) can be inserted or integrated at a desired site or locus within a genome. The insertion sequence of the donor template can comprise a transgene or construct, such as a designed element or a tissue-specific promoter. The donor molecule can also have one or two homology arms flanking the insertion sequence to promote the targeted insertion event through homologous recombination and/or homology-directed repair. Thus, a recombinant DNA molecule of the present application can further include a donor template for site-directed or targeted integration of a transgene or construct, such as a transgene or transcribable DNA sequence encoding a designed element or a tissue-specific promoter into a genome.

As used herein, an “allele” refers to a variant of a given locus or gene in a genome. If the same allele is present on both chromosomes of a chromosome pair in a cell the cell is considered homozygous at the given locus. If each member of the chromosome pair comprises a different allele for the given locus the cell is heterozygous for the locus. A minimum of one allele is possible for a given locus, although typically multiple alleles are possible for any given locus in a genome.

As used herein a “donor molecule” is defined as a molecule comprising a nucleic acid sequence designed or selected for site directed, targeted incorporation into a genome. In one aspect, a genome editing system provided herein comprises the use of one or more, two or more, three or more, four or more, or five or more donor molecules. A donor molecule provided herein can be of any length. For example, a donor molecule provided herein is between 2 and 50,000, between 2 and 10,000, between 2 and 5000, between 2 and 1000, between 2 and 500, between 2 and 250, between 2 and 100, between 2 and 50, between 2 and 30, between 15 and 50, between 15 and 100, between 15 and 500, between 15 and 1000, between 15 and 5000, between 18 and 30, between 18 and 26, between 20 and 26, between 20 and 50, between 20 and 100, between 20 and 250, between 20 and 500, between 20 and 1000, between 20 and 5000 or between 20 and 10,000 nucleotides in length. A donor molecule can comprise one or more genes that encode actively transcribed and/or translated gene sequences. Such transcribed sequences can encode a protein or a non-coding RNA. In one aspect, the donor molecule can comprise a polynucleotide sequence which does not comprise a functional gene or an entire gene (e.g., the donor molecule can simply comprise regulatory sequences such as a promoter), or does not contain any identifiable gene expression elements or any actively transcribed gene sequence. Further, the donor molecule can be can be linear or circular, and can be single-stranded or double-stranded. It can be delivered to the cell as naked nucleic acid, as a complex with one or more delivery agents (e.g., liposomes, poloxamers, T-strand encapsulated with proteins, etc.) or contained in a bacterial or viral delivery vehicle, such as, for example, Agrobacterium tumefaciens or a geminivirus, respectively. In another aspect, a donor molecule provided herein is operably linked to a promoter. In a still further aspect, a donor molecule provided herein is transcribed into RNA. In another aspect, a donor molecule provided herein is not operably linked to a promoter.

In an aspect, a donor molecule provided herein can comprise at least one, at least two, at least three, at least four, at least five, at least six, at least seven, at least eight, at least nine, or at least ten genes. In an aspect, a donor molecule provided herein comprises no genes. Without being limiting, a gene provided herein can include an insecticidal resistance gene, an herbicide tolerance gene, a nitrogen use efficiency gene, a water use efficiency gene, a nutritional quality gene, a DNA binding gene, a selectable marker gene, an RNAi construct, a site-specific genome modification enzyme gene, a single guide RNA of a CRISPR/Cas9 system, a geminivirus based expression cassette, or a plant viral expression vector system. In one aspect, a donor molecule comprises a polynucleotide that encodes a promoter. In another aspect, a donor molecule provided herein comprises a polynucleotide that encodes a tissue-specific or tissue-preferred promoter. In still another aspect, a donor molecule provided herein comprises a polynucleotide that encodes a constitutive promoter. In another aspect, a donor molecule provided herein comprises a polynucleotide that encodes an inducible promoter. In another aspect, a donor molecule comprises a polynucleotide that encodes a structure selected from the group consisting of a leader, an enhancer, a transcriptional start site, a 5′-UTR, an exon, an intron, a 3′-UTR, a polyadenylation site, a transcriptional termination site, a promoter, a full-length gene, a partial gene, a gene, or a non-coding RNA. In one aspect, a donor molecule provided herein comprises one or more, two or more, three or more, four or more, or five or more designed elements.

### EXAMPLES

### Example 1: Design of a Tether Guide Oligo (tgOligo)

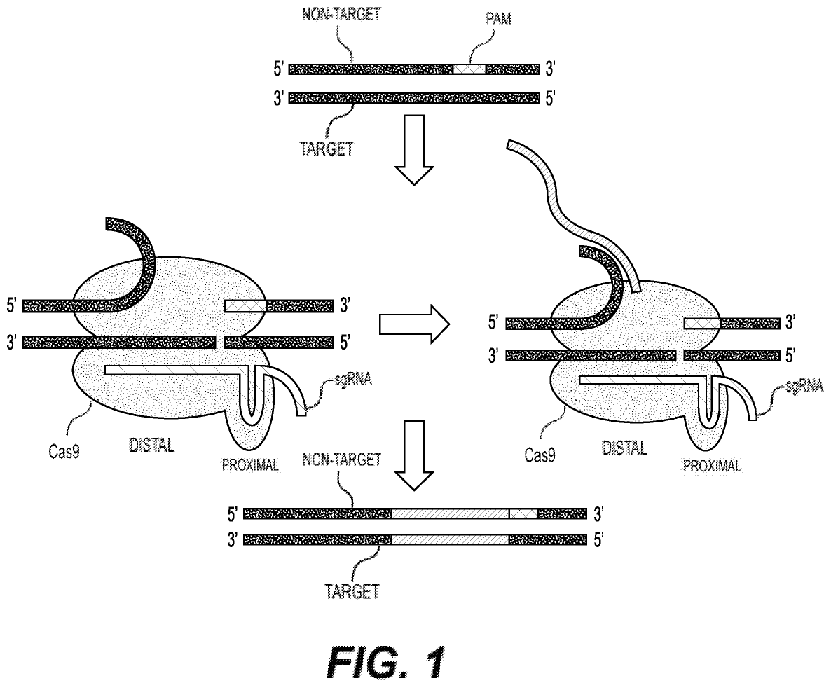

A Cas9/sgRNA complex binds to a dsDNA molecule comprising target and non-target strands. Cas9-PAM interaction occurs on the non-target strand; sgRNA-DNA annealing occurs on the target strand. RuvC (His840) and HNH (Asp10) nuclease domains cut the non-target and target strands, respectively. The blunt ends at the Cas9 cut site are held in place by Cas9 at the 5′ end of the non-target strand (PAM location), and at both cut ends (3′ and 5′) of the target strand. The 3′ cut end of the non-target strand is free and ‘flaps’ around. The 3′ free ‘flap’ end of the non-target strand can be up to 35 nucleotides which is sufficient for specific complementarity binding. A tgOligo (e.g., a ssDNA molecule commentary to the 3′ free ‘flap’ end) is designed and can serve as a template for integration of desired nucleotide modifications (FIG. 1). A tgOligo can be DNA, RNA, or a mix of nucleotides depending on the need and design of the edits. In the case of nucleases (e.g., Cpf1) that provide overhangs from a double-stranded break (DSB) cut, the overhangs can act in place of, or in conjunction with, tgOligos.

### Example 2: Engineering of Cas9-Like Nuclease

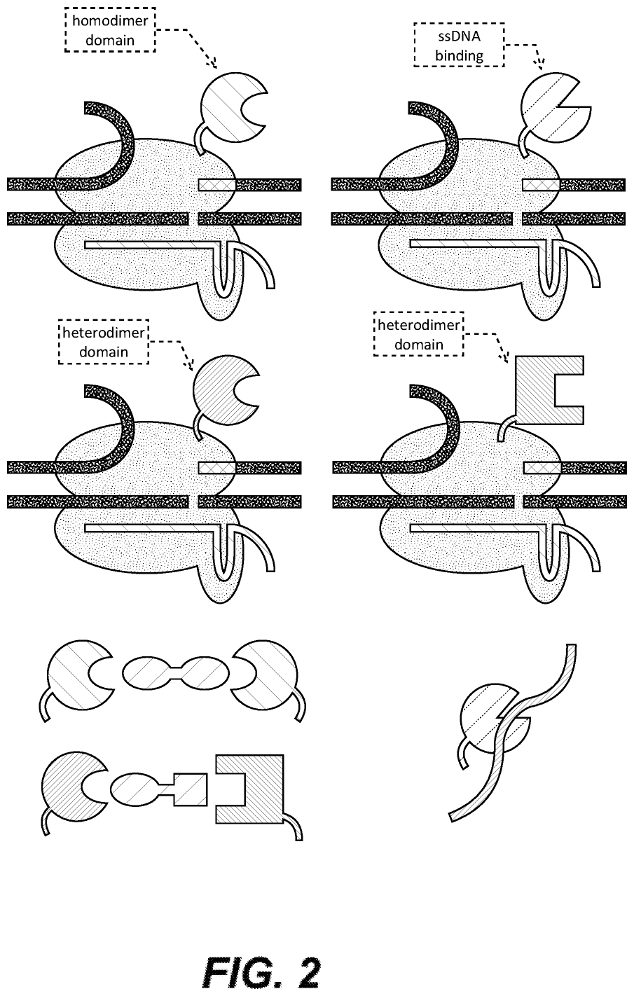

Nucleases, such as Cas9, can be repurposed for structural and functional genomics in plants. Various dimerization domains or ssDNA binding domains can be conjugated to Cas9 to achieve dimerization (e.g., FIG. 2). For example, inducible dimerization domains from Clonetech's homodimerization or heterodimerization iDimerize system can be used to achieve Cas9 dimerization. Alternatively, ssDNA binding domains from Affymetrix or NEB can also be attached to a nuclease (e.g., Cas9) to facilitate dimerization. Additional dimerization systems can also be used such as those described in Andersen et al., Scientific Reports 6, Article number: 27766 (2016); and Miyamoto, et al., Nature Chemical Biology 8(5):465-70(2012).

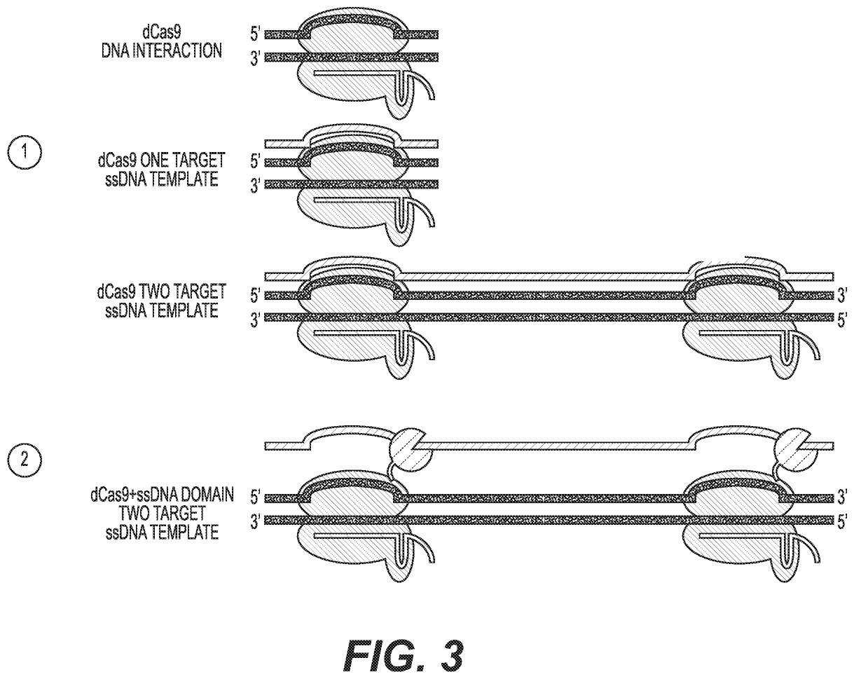

Nucleases, such as Cas9, can also be engineered to form a catalytically deactivated from, such catalytically deactivated Cas9 (dCas9). dCas9 binds to DNA at a target site specified by a gRNA and creates a loop structure accessible for template-based editing (FIG. 3, Panel 1). dCas9 can be further modified to form a fusion with a ssDNA binding domain for further facilitating template-based editing (FIG. 3, Panel 2). The editing efficiency with this modified dCas9-ssDNA binding scheme is expected to be higher compared to a dCas9-alone approach, because a ssDNA template is bound to dCas9 complex and would be brought into proximity of the target site specified by the gRNA.

### Example 3: Introduction of Multiple tgOligo and gRNA Molecules

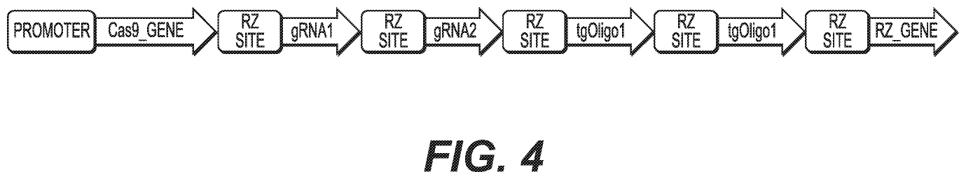

Multiple approaches can be used to incorporate tgOligos with editing components (e.g. nuclease, gRNA). tgOligos can be incorporated in any manner available to deliver nucleases and gRNAs (transfection, transformation, etc). The optimal approach depends on the editing component delivery system and the target organism to be edited. For example, in mammalian systems where RNPs (ribonucleoproteins—complexes of nuclease and gRNA) can be transfected across the cell membrane, tgOligos can be simultaneously transfected. Alternatively, a single transcription unit (STU) can be used to incorporate the nuclease (e.g., Cas9 or Cpf1) and gRNAs in the same transgene construct. Similarly, tgOligos can be incorporated in a similar design (e.g., FIG. 4). Multiple constructs could also be used, such as one for the nuclease, one for the gRNAs, and one for tgOligos—or any combination thereof from inclusive in constructs to combining constructs and transfection delivery. For tgOligos included in constructs (such as FIG. 4), these would be RNA-based tgOligos. To utilize DNA-based or mixed nucleotide (DNA+RNA) tgOligos, a transfection or other delivery mechanism would likely be needed. Also if any tgOligo designs result in the tgOligo containing the same gRNA+PAM recognition site as the original gRNA target site, the tgOligo sequence can be modified to eliminate the PAM to prevent cleavage by the CRISPR nuclease.

### Example 4: Genome Editing Based on a Two-gRNA Approach

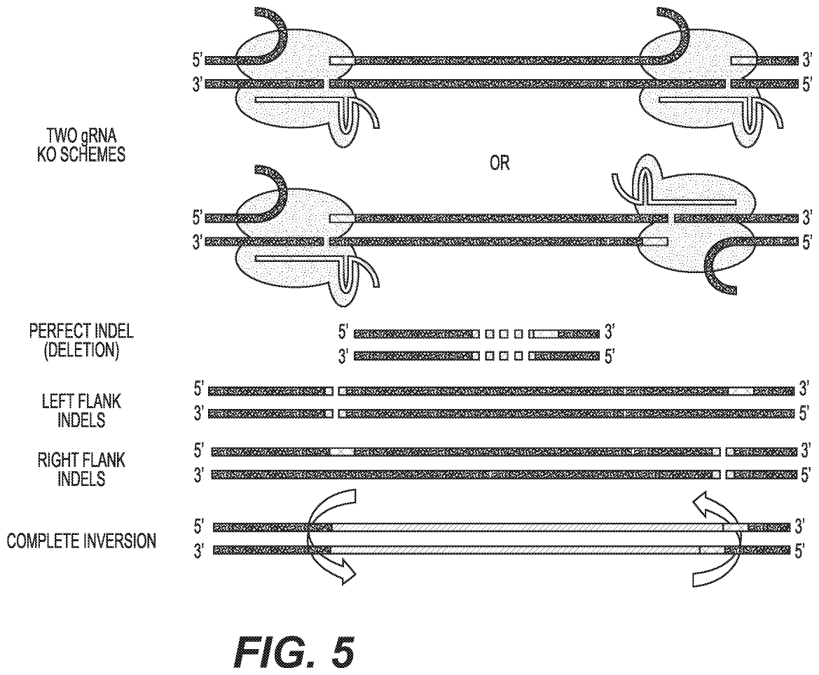

Two Cas9/gRNA complexes flanking a target genomic region are designed for achieving INDELs or complete inversion of the flanked target genomic region. (FIG. 5). Not wishing to be bound by a particular theory, with two Cas9/gRNA complexes, the flanked genomic region is deleted and NHEJ repair combines the two cut sites back together. INDEL (insertion/deletion) mutations may occur at either Cas9/gRNA flanking site. It is also possible to recover with lower frequency complete inversions of the flanked genomic region.

### Example 5: Enhancement of the Two-gRNA Approach

The two-gRNA approach from Example 4 is modified to improve genome editing efficiency. Using dimerization domains (See FIG. 2), tgOligos (See FIG. 1), or combinations thereof can enhance recovery of complete knockout (deletion) of the genomic region flanked by the two gRNA target sites (FIG. 6). Panel 1 of FIG. 6 shows a dimerization-enhanced knock out (KO) event. Panel 2 of FIG. 6 shows a tgOligo-enhanced KO event. Panel 3 of FIG. 6 shows an enhanced KO event via a combination of dimerization and tgOligos. Panel 4 of FIG. 6 shows a tgOligo-enhanced inversion event. Without being bound to any theory, tgOligos can facilitate recovery of inversion events by using complementarity of the tether portions to the opposite end of the flanked segment. The tgOligos can vary in length for the complementation to the 3′ flap of the non-target strand as well as the template tether extension beyond the flap complementation.

Paired dimerization domains coupled with active or inactivated site-specific nucleases (e.g., Cas9, dCas9, Cpf1, dCpf1, etc.) (either alone or in conjunction with tgOligos) can also be used to facilitate inversion of flanked sequence target. Panel 5 of FIG. 6 shows a dimerization-enhanced inversion event. Panel 6 of FIG. 6 shows an inversion event assisted by a combination of Cas9 dimerization/deactivation and tgOligos.

### Example 6: Genomic Knockout of Corn Y1 Gene Using Enhanced Two-gRNA Approaches

The various enhanced two-gRNA approaches described in Example 5 are used to edit the Y1 gene in corn. A reference Y1 gene sequence (GRMZM2G300348_T02) is set forth as SEQ ID NO:1. Two gRNA target sites are chosen. One is in the sense strand at the proximal end of Y1 (SEQ ID NO:2); the other is in the antisense strand at the distal end of Y1 (SEQ ID NO:3). Two gRNAs are designed with a Streptococcus pyogenes Cas9 PAM (NGG) for corn with up to 10 off-targets allowed.

First, a Cas9 dimerization-based approach (illustrated in Panel 1 of FIG. 6) is used in conjunction with the two Y1 gRNAs described above. Inducible dimerization domains from Clonetech's iDimerize system can be used to achieve Cas9 dimerization.

Second, a tgOligo-based approach (illustrated in Panel 2 of FIG. 6) is used to achieve a high efficiency knockout. tgOligos can be all DNA, all RNA, or a mixture of DNA/RNA nucleotides. As an illustrative example, RNA-only tgOligos are described below. Without being bound to any theory, RNA tgOligos are removed when a target site is repaired resulting in a desired knockout without integration of the tgOligos into the genome. If tgOligos include DNA sequence, it can be incorporated during the site repair—which is a desired feature in some of the editing schemes described below (e.g., template editing, Site-directed integration (SDI), facilitated recombination).

A sense strand RNA tgOligo is designed to complement the sense strand flank gRNA target site, generally about 20 bp long. Optionally, a 20 bp segment upstream of the target site is added. An example of a Y1 sense strand RNA tgOligo comprises a DNA-complementary section as set forth in SEQ ID NO:4, which is complementary to SEQ ID NO:2 with 10 bp included from upstream. SED ID NO 4 is reversed to orient 5′-3′ as set forth in SEQ ID NO:5 which is then subsequently converted to an RNA sequence (SEQ ID NO:6). For the final sense strand tgOligo RNA, a 30 bp random RNA sequence is added to the end of SEQ ID NO:6. This random RNA sequence functions as the tether to complement with the antisense strand tgOligo to facilitate the DSB repair across the targeted segment for deletion. An example of the random 30 bp RNA sequence is shown in SEQ ID NO:7 which is added to SEQ ID NO:6 on the 5′ end. This gives rise to a final sense strand tgOligo (SEQ ID NO:8).

An antisense strand RNA tgOligo is designed following the following procedure. Initially, a 20 bp sequence is taken from the antisense strand flank gRNA target site. Optionally, a 20 bp sequence downstream of the target site is also included. An example of a Y1 antisense strand RNA tgOligo comprises a DNA-complementary section as set forth in SEQ ID NO:9, which complements to SEQ ID NO:3 with 10 bp included from downstream. SEQ ID NO:9 is then converted from DNA to RNA (SEQ ID NO:10). A reverse complement to the random 30 bp RNA tether (SEQ ID NO:7), as shown in SEQ ID NO:11, is then used as the tether for the antisense strand tgOligo. SEQ ID NO:11 is attached to SEQ ID NO:10 on the 5′ end to form a final antisense strand tgOligo (SEQ ID NO:12).

Third, a combined enhancement approach is tested that combines both tgOligos and Cas9 dimerization (as illustrated in Panel 3 of FIG. 6). The same tgOligos (SEQ ID NOs: 8 and 12) can be used with the Y1 gRNAs (SEQ ID NOs: 2 and 3) in conjunction with Cas9-dimerization domain complexes. Different tgOligos can be used as well.

Corn plants are transformed using a transfer DNA (T-DNA)-based approach using Agrobacterium. A T-DNA construct comprises one or more plant-expressible promoters operably linked to sequences encoding a genome editing system described here (e.g., a Cas9 nuclease (or a modified version with a dimerization domain), two gRNAs, one or more tgOligos) between a left border (LB) sequence and a right border (RB) sequence. Immature corn embryos are co-cultured with Agrobacterium containing a desired T-DNA vector for three days. Regenerated plantlets are selected on glyphosate containing medium and then subsequently transferred to soil in a growth room.

### Example 7: Genome Editing of Corn BR2 Gene by a tgOligo-Assisted Genomic Inversion Approach

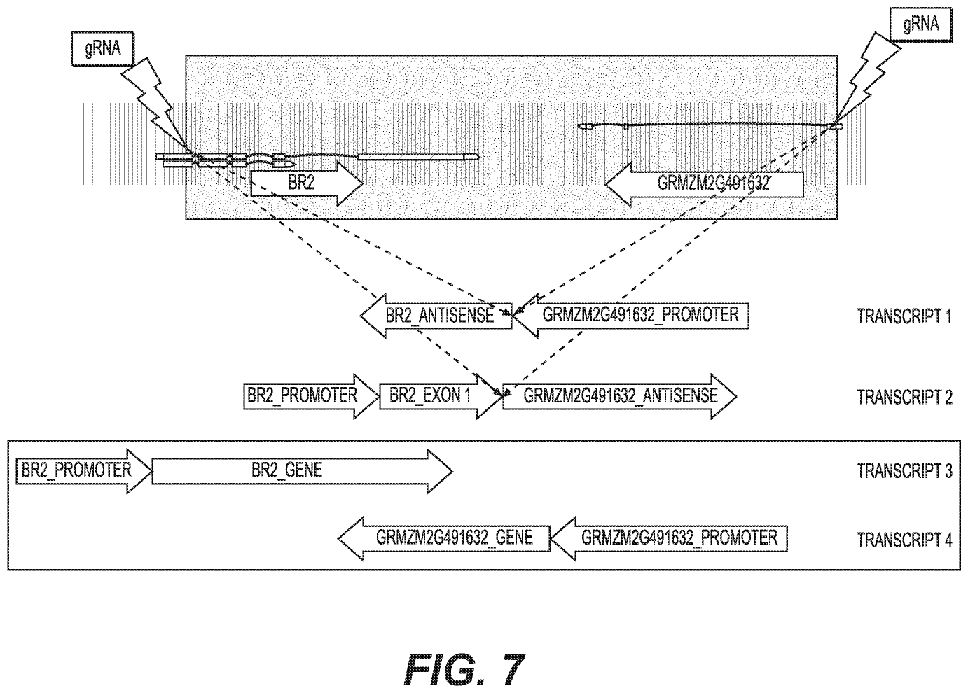

A tgOligo-assisted inversion approach (as illustrated in Panel 4 of FIG. 6) is used to edit the corn BR2 gene to generate a dominant knockout (KO) mutant allele. The rationale for a genome inversion-based dominant KO mutation approach is depicted in FIG. 7. In essence, two gRNAs are used. A first gRNA (shown on the left) targets the end of the first exon of BR2; a second gRNA (shown on the right) recognizes the start codon region of the adjacent GRMZM2G491632 gene. Inversion of the genomic segment flanked by these two gRNAs can lead to a BR2 antisense partial transcript (See Transcript 1). This BR2 antisense transcript is produced via the GRMZM2G491632 promoter activity. Adjusting the relative position of the two gRNAs can achieve a BR2 antisense complete transcript (e.g., moving the first gRNA on the left to target the start codon region of the BR2 gene) or a BR2 antisense transcript under the control of the native BR2 promoter (e.g., moving the second gRNA on the right to target the stop codon region of the BR2 gene).

Reference sequences are listed in SEQ ID NO:13 for BR2 (NCBI accession AY366085) and SEQ ID NO:14 for GRMZM2G491632 (from MaizeGDB). GRMZM2G491632 is a gene annotated immediately adjacent to BR2; and these two genes are in reverse orientation of each other. SEQ ID NO:15 is the gRNA to the sense strand at the proximal end of BR2. SEQ ID NO:16 is the gRNA to the antisense strand at the proximal end of GRMZM2G491632.

A first RNA tgOligo corresponding to the BR2 gRNA (SEQ ID NO:15) is designed to complement the sense strand flank gRNA target site, generally about 20 bp long. Optionally, a 20 bp segment upstream of the target site is added. An example of a BR2 RNA tgOligo comprises a DNA-complementary section as set forth in SEQ ID NO:17 (serving as a DSB 3′ flap complement region), which is complementary to SEQ ID NO:15 with 10 bp included from upstream. Next, a sequence having at least 20 bp starting with the first base of the PAM of the antisense strand gRNA (SEQ ID NO:16) is selected to give rise to a 50 bp sequence including the PAM (SEQ ID NO:18, serving as a tether region). Subsequently, the 3′ flap complement (SEQ ID NO:17) is reversed and attached to the end of the tether (SEQ ID NO:18) to form a complete tgOligo which complements both the sense gRNA and template from antisense gRNA segment for inversion (SEQ ID NO:19).

A second RNA tgOligo corresponding to the GRMZM2G491632 gRNA (SEQ ID NO:16) is designed as follows: a) from the reference sequence (SEQ ID NO:14) reverse complement the antisense strand flank gRNA target site; b) select at least 20 bp starting with the first base of the PAM of the sense strand gRNA (SEQ ID NO:15) and reverse complement. This example is 50 bp including the PAM (SEQ ID NO:21); c) attach the 3′ flap complement (SEQ ID NO:20) to the end of the tether (SEQ ID NO:21) to complete the tgOligo design complementing the sense gRNA and template from antisense gRNA segment for inversion (SEQ ID NO:22).

A combination of two gRNAs and the first and second tgOligos are used to edit the corn BR2 locus to achieve a genomic inversion. The resulting inversion of BR2 and GRMZM2G491632 is expected to form a sequence with high similarity (95%+) to SEQ ID NO:23.

### Example 8: Enhancement of Template-Based Genome Editing or Site Directed Integration (SDI)

Nuclease dimerization or deactivation, tgOligos, or their combination can be used to enhance targeting of template-based editing or site directed integration (SDI) at a single location or multiple locations. Various representative embodiments are depicted in FIG. 8. In these embodiments, a template molecule (regardless of its homology to a target site in the genome) is brought into proximity with the target site by nuclease (e.g., Cas9, Cpf1, TALEN, ZFN) complexes with dimerization domains (Panels 1 to 3 of FIG. 8), tgOligos (not illustrated alone), or a combination thereof (Panel 4 of FIG. 8). dCas9 can be used on the template (Panel 1 of FIG. 8) or active Cas9 can help facilitate integration of the template (Panels 2 to 4 of FIG. 8). The corn Y1 gene reference sequence (SEQ ID NO:24) is used below to demonstrate the concepts in FIG. 8. This Y1 reference sequence (SEQ ID NO:24) is GRMZM2G300348_T01 related to the Y1 reference sequence already provided (SEQ ID NO:1) GRMZM2G300348_T02.

### Example 9: Genome Editing of Corn Y1 Gene to Generate Dominant Alleles

The embodiments of enhanced genome editing depicted in FIG. 8 are tested by creating a dominant allele for a traditionally recessive trait. The following is a summary of the molecular designs for the corn Y1 gene (SEQ ID NO:24).

For Y1, the first exon from SEQ ID NO:24 is shown in SEQ ID NO:25. To make an antisense template, SEQ ID NO:25 is reverse complemented into SEQ ID NO:26 which is used as a template sequence for editing (corresponding to the template sequences between the dCas9 complexes and Cas9 complexes depicted in FIG. 8's Panels 1 and 2). The sense strand gRNA for Y1 (SEQ ID NO:24) is in the 5-UTR (SEQ ID NO:27). The antisense strand gRNA for Y1 (SEQ ID NO:24) is in the 3-UTR (SEQ ID NO:28). The region between these two gRNAs corresponds to the to-be-replaced genomic sequences between the Cas9 complexes depicted in FIG. 8's Panels 1, 2, and 4.

To provide a template for integration (as depicted in FIG. 8's Panels 1 and 2), SEQ ID NO:26 is added between the gRNA target sites (SEQ ID NOs: 27 and 28). The resulting SEQ ID NO:29 comprises the sense strand gRNA site with 10 bp upstream, SEQ ID NO:26, and the antisense strand gRNA site with 10 bp downstream.

This template molecule (SEQ ID NO:29) is then paired with gRNAs (SEQ ID NOs: 27 and 28) and used in editing following the schemes depicted in FIG. 8's Panels 1 and 2. To utilize tgOligos to further help facilitate integration of the template (SEQ ID NO:29) (See Panel 4 of FIG. 8), two tgOligos are incorporated (SEQ ID NOs: 30 and 31).

### Example 10: Genome Editing of Corn BR2 Gene to Generate Dominant Alleles

The enhanced genome editing methods depicted in FIG. 8 are also tested in creating a dominant allele for corn BR2 gene (SEQ ID NO:13). The following is a summary of the molecular designs for BR2.

New gRNAs are designed to be able to replace the BR2 gene with an antisense template similar to the Y1 concept described above. A sense strand gRNA is shown in SEQ ID NO:32 (bold text) and the antisense strand gRNA is shown in SEQ ID NO:33 (bold red text). The region between these two gRNAs corresponds to the to-be-replaced genomic sequences between the Cas9 complexes depicted in FIG. 8's Panels 1, 2, and 4.

The first 250 bp coding sequence of the BR2 gene (SEQ ID NO:34) is made into an antisense template. SEQ ID NO:34 is reverse-complemented to create BR2 Exon 1 antisense sequence template (SEQ ID NO:35).

To provide a template for integration (as depicted in FIG. 8's Panels 1 and 2), SEQ ID NO:35 is added between the gRNA target sites (SEQ ID NOs: 32 and 33). SEQ ID NO:36 comprises the sense strand gRNA site with 3 bp upstream, SEQ ID NO:35, and the antisense strand gRNA site with 10 bp downstream.

This template molecule (SEQ ID NO:36) is then paired with gRNAs (SEQ ID NOs: 32 and 33) and used in editing following the schemes depicted in FIG. 8's Panels 1 and 2. To utilize tgOligos to further help facilitate integration of the template (SEQ ID NO:36) (See Panel 4 of FIG. 8), two tgOligos are incorporated (SEQ ID NOs: 37 and 38).

The examples shown above for editing Y1 and BR2 corn genes can be followed to design neighboring template edits or integrations as illustrated in Panel 3 of FIG. 8. While the examples provided for Y1 and BR2 use antisense templates of the first exon of these genes, the template integrations could be more subtle, such as changing nucleotides to alter amino acids in the native proteins, or more complex such as integrating a non-native sequence or gene. This is further illustrated in FIG. 9.

A potential advantage to creating antisense templates in the native genomic region of Y1 and BR2 as described above is that the native promoter and gene expression elements are used to regulate the antisense transcript to appropriately achieve gene silencing of a native allele in a heterozygous organism.

### Example 11: Template-Based Editing, Site Directed Integration, and Recombination Assisted by tgOligos

The tgOligo concept are used to provide template sequences to repair or integrate between flanked nucleases as illustrated in FIG. 9 and FIG. 10. Here, a template sequence is a portion of a tgOligo (referred to as tgOligo template). A tgOligo template can be used to recover the same size flanked-segment (Panel 1), a smaller segment (Panel 2), or larger segment (Panel 3) depending on the designed edit. To facilitate recombination, a tgOligo template sequence can be identical to the native reference sequence and the Cas9 complexes can be on separate chromosomes (See the following three figures). A tgOligo template sequence can introduce native or non-native sequences to the target site.

Additionally, tgOligos can be further coupled with double-strand oligos (dsOligos) to enhance template-based genome editing or site directed integration (FIG. 11). Here, dsOligos with complementary overhangs and further complementarity with tgOligos can be used to form a larger template for site directed integration or editing.

For the schemes depicted in FIG. 9, Cas9 complexes with dimerization domains can also be used (not illustrated), and it would be expected that tgOligo templates can form a hairpin-type structure as they complement and are integrated into the genomic target site.

The example provided in FIG. 8, Panel 4 can also be used for concept in FIG. 9, Panel 2. For the Y1 and BR2 genes, the genomic sequence of the genes are replaced with a smaller template sequence. The difference between FIG. 8, Panel 4 and FIG. 9, Panel 2 is that the former has two separate molecules as template and tgOligo, while the latter has the two components in the same molecule (e.g., a tgOligo template). For Y1, example gRNAs are SEQ ID NOs: 27 and 28 and examples tgOligos are SEQ ID NOs: 30 and 31. For BR2, examples gRNAs are SEQ ID NOs: 32 and 33 and examples tgOligos are SEQ ID NOs: 37 and 38.

The same principles for FIG. 8, Panel 4 and FIG. 9, Panel 2 can also be used to facilitate the use of tgOligos to integrate a genomic segment equivalent to the flanked region (FIG. 9, Panel 1) or larger than the flanked region (FIG. 9, Panel 3) depending on the desired edit to be achieved.

### Example 12: Enhanced Genome Editing for Achieving Cis Chromosome Arm Exchange

The same concepts illustrated in FIG. 7 to FIG. 9 can be applied with nuclease complexes targeted to different chromosomes, which is shown in FIG. 12 to facilitate a chromosome arm exchange. Dimerization domains can bring Cas9/gRNA complexes in proximity to facilitate DNA repair exchanging two chromosome arms (Panels 1, 3, and 4). Inclusion of tgOligos can further facilitate recombination at the site (Panels 2 to 5). Either cis or trans chromosome arm exchange is illustrated in FIG. 12 using dimerization domains (Panel 1), tgOligos (Panel 2), a dimerization/tgOligo combination at the same site (Panel 3) or at different sites (Panel 4), and with ssDNA binding domains combined with hairpin tgOligos (Panel 5).

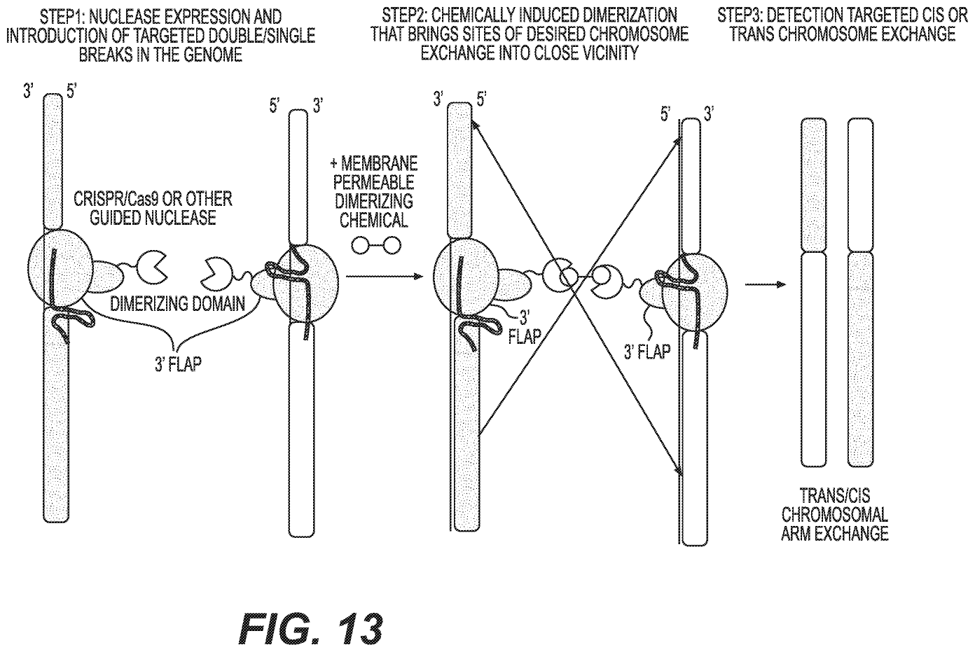

FIG. 13 further illustrates the use of induced homo or hetero dimerization technology to facilitate targeted chromosome arm exchange. Dimerization can be induced by chemicals, light, or other stimulants.

Without being bound to any theory, Cas9/gRNA complexes on sister chromosomes can make DSB and have NHEJ repairs result in chromosome arm exchanges. The expected frequency of this occurrence is likely low. To facilitate a guided or directed NHEJ repair and achieve a chromosome arm exchange, dimerization domains on the nuclease and/or tgOligos on the 3′ free flap in the nuclease complex that align together and bring the chromosome arms into a crossing over recombination (FIG. 12). A templated insertion (FIG. 9) can also be incorporated with the exchange/recombination too (not illustrated).

### Example 13: Editing of the Corn BR2 Gene Via Chromosome Arm Exchange

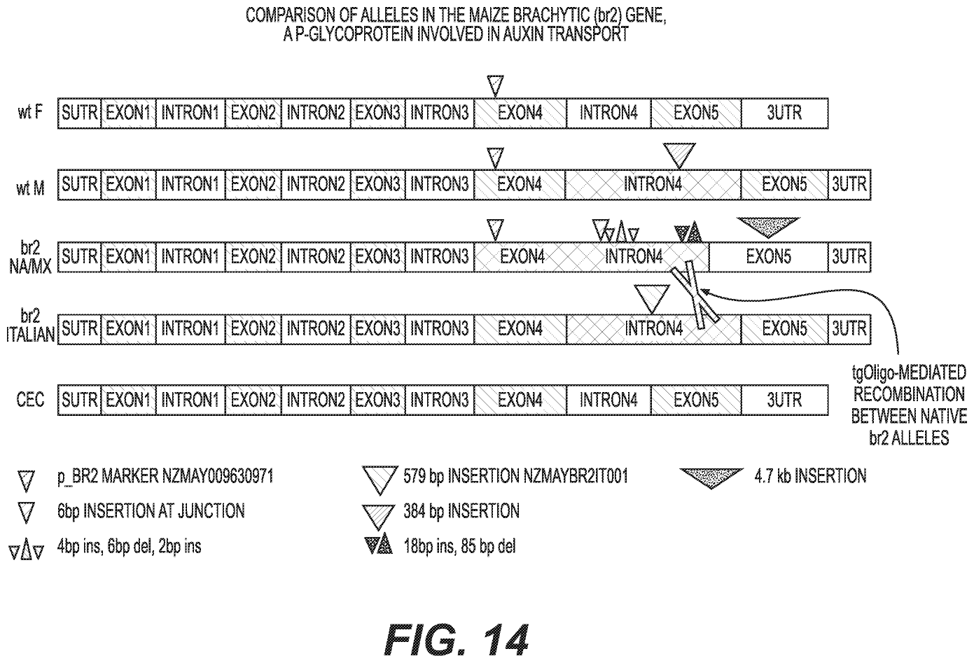

The concepts depicted in FIG. 12 are tested in editing the BR2 gene (SEQ ID NO:13). Two native br2 mutant alleles are identified (FIG. 14). One allele carries an INDEL mutation in Intron 4 (br2-Italian) and the other allele carries an INDEL mutation in Exon 5 (br2-NA/MX). The distance between the genomic position of these two INDEL mutations is <1000-2000 bp. It would take screening a large population to identify a recombination event in this region to stack the two INDEL mutations in cis on the same chromosome. The genome editing schemes illustrated in FIG. 12 can be used to recover this rare recombinant more efficiently.

The br2-NA/MX allele carries a 4.7 kb insertion (triangle) in Exon 5. The br2-Italian allele carries a 579 bp insertion Intron 4 (triangle). Example tgOligos are designed as described below to facilitate a specific recombination between these two insertions to stack them on the same chromosome. A homozygous inbred with the br2-NA/MX allele could be crossed to a homozygous inbred with the br2 Italian allele to create an Fi in the presence of a genome editing machinery including tgOligos to facilitate the recombination.

Two approaches are designed to illustrate possible tgOligo-mediated recombination at BR2's Intron 4 location (SEQ ID NO:42) to achieve recombination between br2-NA/MX and br2-Italian.

In a first approach, two gRNAs with tgOligos are designed which are spaced apart from each other. SEQ ID NO:39 is the gRNA for the left flank (sense strand) and SEQ ID NO:40 is the gRNA for the right flank (antisense strand). SEQ ID NOs: 43 and 44 are the tgOligos to pair with these gRNAs. The tether sequence in SEQ ID NOs: 43 and 44 is the native template of BR2 Intron 4 between the flanking gRNAs. A recombination facilitated by these tgOligos would result in the native template sequence remaining between the gRNAs since it was provided as the tethering sequence in the tgOligos.

In a second approach, two gRNAs that have head-to-tail PAM sequences with tgOligos are designed with DNA complement sequence to the 3′ free flap and RNA sequence tether to bind the tgOligos facilitating recombination. SEQ ID NO:41 is the gRNA for the sense strand (head) and SEQ ID NO:40 is the gRNA for the antisense strand (tail). SEQ ID NOs: 45 and 46 are the tgOligos to pair with these gRNAs. The tether sequence in SEQ ID NOs: 45 and 46 is a randomly generated RNA nucleotide sequence (SEQ ID NO:7). To test the scheme illustrated in Panel 4 of FIG. 12, a gRNA sequence (SEQ ID NO:47) is used together with dCas9/dimerization to achieve BR2 recombination. A recombination facilitated by these tgOligos can result in double-strand break repair at the head to tail PAM sequences with no incorporation of the RNA tethering sequences. As shown in Panel 1 of FIG. 12, tgOligos may not be required to create this recombination by using dimerized nucleases alone. Example 14: Enhanced genome editing for achieving cis or trans genomic fragment exchange.

The various tgOligo/dimerization/deactivation-based genome editing enhancement approaches can be used to facilitate cis or trans genomic fragment exchange. FIG. 15 depicts a cis genomic fragment exchange approach using dimerization domains (Panel 1), tgOligos (Panel 2), a dimerization/tgOligo combination at the same site (Panel 3) or at different sites (Panel 4). The same concepts from FIG. 12 and earlier are applied to flank a genomic segment on homologous (cis) chromosomes and exchange the flanked segment. Dimerization domains, tgOligos, or their combination can enhance the efficiency of the exchange.

Similarly, FIG. 16 illustrates a trans genomic fragment exchange approach using dimerization domains (Panel 1), tgOligos (Panel 2), a dimerization/tgOligo combination at the same site (Panel 3) or at different sites (Panel 4). The same concepts from FIG. 15 and earlier are applied to flank a genomic segment on non-homologous (trans) chromosomes and exchange the flanked segment. Dimerization domains, tgOligos, or their combination can enhance the efficiency of the exchange, especially given the regions would not share homology for native DNA repair facilitation.

### Example 15: Genome Editing in Non-Plant Species

All of the concepts and examples described in this application are not limited to plants despite Y1 and BR2 corn gene examples being provided. The concept of FIG. 16 is tested in cattle to engineer multiple toll-like receptor (TLR) genes into the same chromosome. There are three bovine (cattle) TLR genes that recognize either dsRNA or ssRNA from viruses to initiate innate immunity, TLRs 3, 7, and 8 (Cargill and Womack, Genomics (2007)89:745-55). TLRs 7 and 8 neighbor each other on the X chromosome. TLR3 is on Chr 27.

Example gRNAs and tgOligos are designed to assist in recombining TLR3 with TLRs 7 and 8 on the X chromosome. Combining into the same chromosome all three TLR genes that recognize RNA from viruses can enable more efficient cattle breeding for improved immunity to viral infections.

The following is a summary of the molecular designs for recombining TLR3 with TLRs 7 and 8. The bovine TLR3 reference sequence is SEQ ID NO:48; AC_000184.1:15230174-15245811 Bos taurus breed Hereford chromosome 27, Bos_taurus_UMD_3.1.1, whole genome shotgun sequence. The bovine TLRs 7 and 8 reference sequence is SEQ ID NO:49 with intergenic sequence to target TLR3 recombination; AC_000187.1:c141064591-141002526 Bos taurus breed Hereford chromosome X, Bos_taurus UMD_3.1.1, whole genome shotgun sequence. The target site on the X chromosome between TLRs 7 and 8 is included in SEQ ID NO:50 with the sense strand gRNA (SEQ ID NO:51) and antisense strand gRNA (SEQ ID NO:52). The target site on Chromosome 27 proximal the TLR3 gene is included in SEQ ID NO:53 with the antisense strand gRNA (SEQ ID NO:54). The target site on Chromosome 27 distal the TLR3 gene is included in SEQ ID NO:55 with the sense strand gRNA (SEQ ID NO:56). Without tgOligos and using just nuclease/dimerization domains, SEQ ID NO:51 and SED ID NO 54 would pair together; then SEQ ID NO:52 and SEQ ID NO:56 would pair together. If including tgOligos, SEQ ID NOs: 57 and 58 would help facilitate pairing SEQ ID NOs: 51 and 54. Then tgOligos SEQ ID NOs: 59 and 60 would help facilitate pairing SEQ ID NOs: 52 and 56.

### Example 16: Hairpin Shaped tgOligos and their Combination with Single Strand Binding Domains to Modulate Optimal Stoichiometry for tgOligo Binding

A consideration with the tgOligos and binding components of editing complexes (e.g. Cas9+gRNA) is how to promote the desired complementary binding between the 3′ free flap of the nuclease DSB (double strand break) and the tgOligos. FIG. 18 is an illustration to utilize a molecular beacon or hairpin design (hairpins) approach to the tgOligos. The tgOligos would be in a hairpin formation unless bound to the 3′ free flap of the nuclease DSB. When bound to the 3′ free flap, the tgOligo would be in a single strand form (squiggle line in FIG. 18) accessible to a single strand binding domain that could be attached to the editing complex (purple (pacman shape) in FIG. 18). This can allow the recognition and binding of only tgOligos bound to the DSB junctions so that they are brought together in proximity to facilitate a recombination event. FIG. 18 illustrates these components for a chromosome arm exchange similar to those described in FIG. 12. However, this molecular beacon or hairpin design of tgOligos can apply to any other instances involving tgOligos, e.g., FIG. 15 and FIG. 16.

### Example 17: Use of a Single Molecule Comprising Both sgRNA and tgOligo

A tgOligo can be combined with a sgRNA or two sgRNAs to form a single contiguous molecule. FIG. 19 illustrates the use of such single molecule to facilitate inversion of flanked genomic segment. Here, the tether is a RNA sequence extension of the sgRNA, hence a tgRNA. The 3′ end of the tether would complement the PAM of the opposite side Cas9 complex as shown in FIG. 19. This combined sgRNA+tgRNA molecule could be used to facilitate any of the other approaches described here involving a tgOligo.

### Example 18: Genome Editing-Based Dominant Mutant Allele Via Stacking of an Inverted Y1 Gene Head-to-Tail

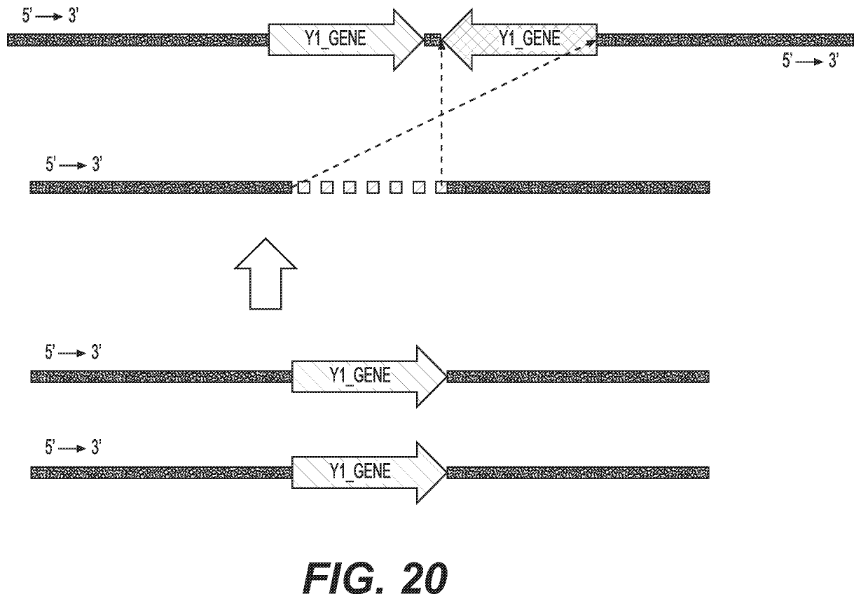

The tgOligo and nuclease dimerization concepts described in the above examples can also be used to stack an inverted gene head-to-tail next to the native copy. This would result in an antisense transcript to silence the gene expression, and therefore create a dominant mutant allele for a normally recessive trait (e.g., the corn Y1 gene, FIG. 20).

### Example 19: A tgOligo-Free Approach to Enhance Chromosomal Translocation

A tgOligo-free approach can be used to link two Cas-mediated double-strand breaks using complementary non-target strand 3′ free flaps (FIG. 21 and FIG. 22). This approach can be used to guide DNA repair to create chromosome exchanges or deletions. Essentially, two gRNAs are designed to cut two genomic locations such that complementary flaps are created. One option is to use two different Cas9 proteins that have different PAM specificities. Then, gRNAs are chosen to target two sites—each with a different PAM. Differences in the spacer target could also be used to produce two complementary flaps.

Alternatively, two gRNAs are designed to cut two genomic locations such that complementary flaps are created. This can be done by designing gRNAs that compete with each other for a shared site. If sequences at both sites are identical, two possible flaps could be produced at each site. Two out of four configurations produce complementary flaps (FIG. 22, Panels 1 and 2). The other two configurations produce identical (not complementary) flaps (FIG. 22, Panels 3 and 4). If sequences are not identical between target sites, then spacers can be designed to only bind one of the two sites and then only complementary flaps would be produced.

### Example 20: A Self-Locked Chimeric tgOligo Approach

A chimeric tgOligo with a hairpin configuration is designed (FIG. 23). The chimeric tgOligo can recognize target sites of two separate gRNAs and bind two separate 3′ free flaps ends generated from DNA cleavage mediated by the two gRNAs. A chimeric tgOligo linking two gRNA target site can be used to promote chromosome translocation. A chimeric tgOligo can also be designed to adopt a hairpin configuration so that it stays in such a configuration until at least a portion of the tgOligo sequence hybridizes with an intended genomic sequence.

1. A genome editing system comprising:
(a) a nuclease or a first nucleic acid encoding said nuclease;
(b) a DNA-targeting guide molecule or a second nucleic acid encoding said DNA-targeting guide molecule,
wherein said DNA-targeting guide molecule and said nuclease form a multi-unit or single-molecule genome editing system;
(c) a tether molecule capable of tethering two entities of said genome editing system, or a third nucleic acid encoding said tether molecule,
wherein said tether molecule is an oligonucleotide-based molecule or a cross-linker heterologous to said nuclease.

2. (canceled)

3. (canceled)

4. The genome editing system of claim 1, wherein said nuclease is a FokI nuclease or an RNA-guided nuclease.

5. (canceled)

6. The genome editing system of claim 1, wherein said nuclease is a Clustered Regularly Interspaced Short Palindromic Repeats (CRISPR) associated nuclease (Cas nuclease) selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1 (also known as Cas12a), and a homolog or modified version thereof.

7.-10. (canceled)

11. The genome editing system of claim 1, wherein DNA-targeting guide molecule is a RNA.

12. The genome editing system of claim 1, wherein DNA-targeting guide molecule is selected from the group consisting of a CRISPR guide RNA, a TAL effector domain, and a zinc finger domain.

13. The genome editing system of claim 1, wherein said tether molecule is selected from the group consisting of a tgOligo, a cross-linker, and a dimerization domain.

14.-45. (canceled)

46. A genome editing system comprising:
(a) a Cas nuclease or a nucleic acid encoding said Cas nuclease;
(b) a first and a second gRNAs or one or more nucleic acids encoding said first and second gRNAs,
wherein a target sequence of said first gRNA and a target sequence of said second gRNA flank a target genomic segment; and
(c) a first tgOligo corresponding to said first gRNA and a second tgOligo corresponding to second gRNA,
wherein said first and second tgOligos are capable of hybridizing with each other.

47. The genome editing system of claim 46, wherein said target sequences of said first and second gRNAs reside on the opposite strands of said target genomic segment.

48. The genome editing system of claim 46, wherein said Cas nuclease is coupled to a cross-linker, and wherein said system further comprises:
(d) a deactivated Cas (dCas) nuclease coupled to a cross-linker, or one or more nucleic acids encoding said dCas nuclease and cross-linker;
(e) a third and a fourth gRNAs or one or more nucleic acids encoding said third and fourth gRNAs,
wherein target sequences of said third and fourth gRNAs are within and on the opposite ends of said target genomic segment; and wherein the dCas nuclease bound to said third or fourth gRNA target sequence is capable of dimerizing with the Cas nuclease bound to a gRNA target sequence on the opposite end of said target genomic segment.

49. The genome editing system of claim 46, wherein said first and second tgOligos are capable of hybridizing and forming a double-stranded template sequence for integration.

50. The genome editing system of claim 49, wherein said double-stranded template sequence is capable of replacing said target genomic segment via said genome editing system.

51. The genome editing system of claim 49, wherein said double-stranded template sequence is longer, shorter, or of equal size compared to said target genomic segment.

52.-60. (canceled)

61. A method for chromosome engineering comprising introducing into a target cell a genome editing system comprising:
(a) a Cas nuclease coupled to a cross-linker or one or more nucleic acids encoding said Cas nuclease and cross-linker, wherein said cross-linker is capable of linking two Cas nuclease molecules; and
(b) a first and a second gRNAs or one or more nucleic acids encoding said first and second gRNAs, and wherein said first and second gRNAs have target sequences in a first recombination region of interest on a pair of donor and recipient chromosomes; and
producing a recombinant chromosome comprising a portion of said donor chromosome and a portion of said recipient chromosome.

62.-65. (canceled)

66. The method of claim 61, wherein said cross-linker comprises a domain selected from the group consisting of a homo-dimerization domain, a hetero-dimerization domain, an inducible dimerization domain, a single-strand DNA binding domain, and an RNA binding domain.

67. (canceled)

68. The method of claim 61, wherein said cross-linker requires a cross-linking ligand.

69. (canceled)

70. (canceled)

71. The method of claim 61, wherein said genome editing system further comprises:
(c) a third and a fourth gRNAs or one or more nucleic acids encoding said third and fourth gRNAs, and wherein said third and fourth gRNAs have target sequences in a second recombination region of interest on said pair of donor and recipient chromosomes;
and wherein said method is capable of producing a recombinant chromosome comprising a backbone from said recipient chromosome with a chromosome segment integrated from said donor chromosome between said first and second recombination regions of interest.

72. (canceled)

73. (canceled)

74. The method of claim 61, wherein said genome editing system further comprises
(c) a first tgOligo corresponding to said first gRNA, a second tgOligo corresponding to said second gRNA, and wherein said first and second tgOligos are capable of hybridizing with each other.

75. The method of claim 74, wherein said genome editing system further comprises:
(d) a third and a fourth gRNAs or one or more nucleic acids encoding said third and fourth gRNAs, and wherein said third and fourth gRNAs have target sequences in a second recombination region of interest on said pair of donor and recipient chromosomes;
(e) a third tgOligo corresponding to said third gRNA, a fourth tgOligo corresponding to said fourth gRNA, and wherein said third and fourth tgOligos are part of a single molecule or are capable of hybridizing with each other;
wherein said method is capable of producing a recombinant chromosome comprising a backbone from said recipient chromosome with a chromosome segment integrated from said donor chromosome between said first and second recombination regions of interest.

76. (canceled)

77. (canceled)

78. The method of claim 61 or 74, wherein said genome editing system further comprises:
(f) a deactivated Cas (dCas) nuclease coupled to a cross-linker, or a nucleic acid encoding said dCas nuclease and cross-linker;
(g) a third and a fourth gRNAs or one or more nucleic acids encoding said third and fourth gRNAs, wherein a target sequence of said third gRNA and a target sequence of said fourth gRNA each reside on one chromosome of said pair of donor and recipient chromosomes, wherein two cross-linked molecules of said dCas nuclease are capable of binding to said third and fourth gRNA target sequences and thereby bringing into close proximity said first recombination region of interest and promoting recombination.

79. The method of claim 78, wherein said genome editing system further comprises:
(h) a fifth and a sixth gRNAs or one or more nucleic acids encoding said fifth and sixth gRNAs, and wherein said fifth and sixth gRNAs have target sequences in a second recombination region of interest on said pair of donor and recipient chromosomes;
(i) a seventh and a eighth gRNAs or one or more nucleic acids encoding said seventh and eighth gRNAs, wherein a target sequence of said seventh gRNA and a target sequence of said eighth gRNA each reside on one chromosome of said pair of donor and recipient chromosomes, wherein two cross-linked molecules of said dCas nuclease are capable of binding to said seventh and eighth gRNA target sequences and thereby bringing into close proximity said second recombination region of interest and promoting recombination;
wherein said method is capable of producing a recombinant chromosome comprising a backbone from said recipient chromosome with a chromosome segment integrated from said donor chromosome between said first and second recombination regions of interest.

80.-112. (canceled)


SEQUENCE LISTING (60 sequences)

SEQ ID NO: 1 - DNA, 4732 bp, Zea mays
gttaattaatagattcatataatacttgatgttgatctatgtgttttatatgcgtctagattcatcttcatctatttgaatatagacataaaaatcaagagctaaaataactactattttggtattttggaatggagggagtaatagacgacaagtgagcctggtgagttacctgaaacaaacaaagccagcagagccagaggtcgcggctatggtagtctgactgccatgcatgtcaccgcgggtgtggggggcgcccagcagccacgtcggacaggagcagccagggtgaatccggccttttccaggtgtcaccactcagcgtcctccgaacacaggagagtcatgcgatgcgagcttggcgataagcttatctatccgcaccgcgtcttccttcctcctgggcgaccggcccttcttctctccacgtctctccccccttctttctccagaccgagcgtacgtatgctacacacagcaacagcacaacagtactagttccaccacaagaagatgcccaatgccaagaaataacccatgcttcttgtcgacgatccagccgcactagagatggccaaacgggccggcccggcccggcccggcccgggcccggtgaagcccggccaaaaccgggccgggcctgctgagccagcgggcttaagtttctgtccaagcccggcccgcagcgggcctaaacaggccgggccggcccgtttagcacgaaaaaacgggccaaaaagcgggctaaacgggccggtaagcacgttttagtgtaaaaaaaacgggcttaacgggcttagaggtaaacgggccgtgccgggctagcccgccgtgcctagtttcctgtccaagcccgcccgcttattctaccgtgccgggctcggaccgggcccaaaaagcgggcttcgtgccgggctcacgggcctcgtgctttttggccatctatgagccgcacacttagcatacatacgcaagaagaggagaggccggaggtgcgcgtgctccttgctgttctgctgactggtctcaccatctcatcccaccaccaccaccaccaccaccatctttaggataagatagcaaatatatggccatcatactcgtacgagcagcgtcgccggggctctccgccgccgacagcatcagccaccaggggactctccagtgctccaccctgctcaagacgaagaggccggcggcgcgccggtggatgccctgctcgctccttggcctccacccgtgggaggctggccgtccctcccccgccgtctactccagcctcgccgtcaacccggcgggagaggccgtcgtctcgtccgagcagaaggtctacgacgtcgtgctcaagcaggccgcattgctcaaacgccagctgcgcacgccggtcctcgacgccaggccccaggacatggacatgccacgcaacgggctcaaggaagcctacgaccgctgcggcgagatctgtgaggagtatgccaagacgttttacctcggtacgtacggtatatatatgggatccatcttcttctccaattccacaatctcatcgtttcagtctgcttccatcactcatgctactagttcgtcgcaggaactatgttgatgacagaggagcggcgccgcgccatatgggccatctatggtatctgtctgtctcaaatacaataatcaccatgcatgtatccctccaatgtatcagtaccattgctcatacctagctagtagcatgttacgtacggagtatcaatcagtaaatttcagaatggctactactggaactggatgcgctgtactagctagtatgtttccctacttaatatataacgtagatacgcacttctgacttaattatgctgctgtggcaaaaggattttttttttttactttaacagcagattccaacaataacagtgcaaaattgggctacttttcaagtaatggtaacaactagcagctcccagtggaaattaacaatattgaaaaaagaaacactgctgacttccatgaaggccattcgtgagtcaatggtgaataaaggtttaatgcgctttatgttgtatgtggaagcaccaaaataggctttcggtagcctaaattgctactaaatggcttgatcaatacttgaagaccatgtggaaagttataagaacatattctttatagttcaaactaccctttaggaatatgaatctcaagtattggcaattttaaacgaacacttcaggcacggtatctgacgggtattgtttctgcagtgtggtgtaggaggacagatgagcttgtagatgggccaaacgccaactacattacaccaacagctttggaccggtgggagaagagacttgaggatctgttcacgggacgtccttacgacatgcttgatgccgctctctctgataccatctcaaggttccccatagacattcaggtactgactgccttacgggctgctgtacctagcggattcattccactgatatgacacttttggactgcagttattgatatttctaaccagcattagattctctagctaggcctcactgtttttagtgcaggatgactagacctactgagttgacaagaagctagcagaaattgttttgtttactcaactgaatcttaagattttttcaacctttagtctcttctagcaatgcttttgtttggttcaatgaaccttgcgcatatcgtagtggtcctagtctagttatatggaccaggacccaggagaggtgtttgggtcaaagctgtttcaatgtaacttttttttaaataaaaaatgaactgtttctagagctcagccctcacagcaaaacttggagcgagaggcaataatttgaatattacatggccttggtaatgtgaattaattagttggtctatccttagttggtccgaccttttaagaaacaaaaggtaactgtatgaccagcgcagaagaaaataggatctagatgatgattgagagagaaacgtttcagggggaaaaaatccaatcaattaaaaattagacctgggaacattggcagatcgatctagagggtggacaaggtgggctgattttgatcctgctaaattatatagatagttttgatttatttgctacttttgattctcatacgttgtagaacttaaaatgtgaactcatttgtttattgattctcataaggttggaccaccttaactttaaatcctagatttgccactgggaagtgaccgagagaaaactatctgtgccacttagtggtttgataaactatgttgtgataccaagttaccaacgttttgaaatcaataaatgttgtggcagccattcagggacatgattgaagggatgaggagtgatcttaggaagacaaggtataacaacttcgacgagctctacatgtactgctactatgttgctggaactgtcgggttaatgagcgtacctgtgatgggcatcgcaaccgagtctaaagcaacaactgaaagcgtatacagtgctgccttggctctgggaattgcgaaccaactcacgaacatactccgggatgttggagaggagtaagtaacatatatattcttcctgcgacaggcacgaacatgcatgtgttcaatagcacagatgtgatgatatgactgtcaccatgtcttttagtgctagaagaggaaggatatatttaccacaagatgagcttgcacaggcagggctctctgatgaggacatcttcaaaggggtcgtcacgaaccggtggagaaacttcatgaagaggcagatcaagagggccaggatgttttttgaggaggcagagagaggggtaactgagctctcacaggctagcagatggccagtaagtccactcaacttcacatttcccacccagtatagcacagcatcctcacttccttttctttgttaccattgcaggtatgggcttccctgttgttgtacaggcagatcctggatgagatcgaagccaacgactacaacaacttcacgaagagggcgtatgttggtaaagggaagaagttgctagcacttcctgtggcatatggaaaatcgctactgctcccatgttcattgagaaatggccagacctagccaccagagaagctgcaatgcaaggttcaggttaggctagatagaaagttaaatggggcaacatcaggaggccttgatgaaaaacagacaacctggtgaattgttgttgggatcaggcacagaacagataagagccgcgcagccaacctagggcatgtttggtttcaattagttctaggactaaactttagtcctaggactaaactttagtccctatatgtttggttctagggactaaatagattctaaagtcattaaatacattgtccaaagactcaaatacccttagaatatactcatgatattagttatctataaaaaggtaagggcaacatgataattatgagcttttagtctcttttagcacctatgtgaaggactaaagactaaatcattttagtccatattttagtcctagtgtttggcaaaaaagggactaaaagggactaaaaactagagactaatctttagtccctctaaccaaacacccccctagatggatacggaacattcgcctcttattcggagcaatatatgtctctcaaggaaagagcccaacatgtatactgccttctttttctcatcccagatttgggggaaaaacaatgtaaatgccaatggtatcgtaggaagattactagaagt

SEQ ID NO: 2 - RNA, 20 nt, Artificial Sequence
  Description of Artificial Sequence Synthetic oligonucleotide
gcucaagacgaagaggccgg

SEQ ID NO: 3 - RNA, 20 nt, Artificial Sequence
  Description of Artificial Sequence Synthetic oligonucleotide
agcgauuuuccauaugccac

SEQ ID NO: 4 - DNA, 30 bp, Zea mays
cgaggtgggacgagttctgcttctccggcc

SEQ ID NO: 5 - DNA, 30 bp, Zea mays
ccggcctcttcgtcttgagcagggtggagc

SEQ ID NO: 6 - RNA, 30 nt, Artificial Sequence
  Description of Artificial Sequence Synthetic oligonucleotide
ccggccucuucgucuugagcaggguggagc

SEQ ID NO: 7 - RNA, 30 nt, Artificial Sequence
  Description of Artificial Sequence Synthetic oligonucleotide
gagcguccaugaugguacucuucaauucac

SEQ ID NO: 8 - RNA, 60 nt, Artificial Sequence
  Description of Artificial Sequence Synthetic oligonucleotide
gagcguccaugaugguacucuucaauucacccggccucuucgucuugagcaggguggagc

SEQ ID NO: 9 - DNA, 30 bp, Zea mays
caccgtataccttttagcgatgacgagggt

SEQ ID NO: 10 - RNA, 30 nt, Artificial Sequence
  Description of Artificial Sequence Synthetic oligonucleotide
caccguauaccuuuuagcgaugacgagggu

SEQ ID NO: 11 - RNA, 30 nt, Artificial Sequence
  Description of Artificial Sequence Synthetic oligonucleotide
gugaauugaagaguaccaucauggacgcuc

SEQ ID NO: 12 - RNA, 60 nt, Artificial Sequence
  Description of Artificial Sequence Synthetic oligonucleotide
gugaauugaagaguaccaucauggacgcuccaccguauaccuuuuagcgaugacgagggu

SEQ ID NO: 13 - DNA, 7139 bp, Zea mays
gctctgccactctgctgaggtggggggagaggagctccccctccctcctctcccctcctcgccatgtctagcagcgacccggaggagatcagggcgcgcgtcgtcgttctcggttcgccccatgccgacggcggcgacgagtgggcccggcccgagctcgaggccttccatctgccgtctcccgcccaccagcctcctggcttcctagccgggcaaccggaagcagcagagcaacccacgctccctgctcctgctggccgcagcagcagcagcagcaacacgcctactacatctgccggtggcggcgctgctcctcctcctccttcttcgcctccccctccgccggcttctctggagaccgagcagccgcccaatgccaggccagcctccgccggcgccaatgacagcaagaagcccaccccgcccgccgccctgcgcgacctcttccgcttcgccgacggcctcgactgcgcgctcatgctcatcggcaccctcggcgcgctcgtccacgggtgctcgctccccgtcttcctccgcttcttcgccgacctcgtcgactccttcggctcccacgccgacgacccggacaccatggtccgcctcgtcgtcaagtacgccttctacttcctcgtcgtcggagcggcaatctgggcatcctcgtgggcaggtacgctatccctcctcctcctgccgccccagcttgtgtgcgtcgcgaattggcggtcaatttggattggatgacaaatcacgtcggtcagccaatcgccgtggctacaaacgagatgttcaaatcgttcgccccgctcgcaagagatctcttgctggatgtggaccggcgagcggcagtcgacgcggatgcggattcggtacctggacgcggcgctgcggcaggacgtgtccttcttcgacaccgacgtgcgggcctcggacgtgatctacgccatcaacgcggacgccgtggtggtgcaggacgccatcagccagaaactgggcaacctcatccactacatggccaccttcgtggccggcttcgtcgtggggttcacggccgcgtggcagctggcgctggtcacgctggccgtggtgccgctcatcgccgtcatcggcgggctgagcgccgccgcgctcgccaagctctcgtcccgcagccaggacgcgctctcgggcgccagcggcatcgcggagcaggcgctcgcgcagatacggatcgtgcaggcgttcgttggcgaggagcgcgagatgcgggcctactcggcggcgctggccgtggcgcagaggatcggctaccgcagcggcttcgccaaggggctcggcctcggcggcacctacttcaccgtcttctgctgctacgggctcctgctctggtacggcggccacctcgtgcgcgcccagcacaccaacggcgggctcgccatcgccaccatgttctccgtcatgatcggcggactgtaaggcccaccacaccacgcactctctccttctgctgtcctcggccgcccccgtcgtcattgctgctgacggtatctgtggatcgcgtgcaggcctcggcagtcggcgccgagcatggccgcgttcgccaaggcgcgtgtggcggctgccaagatcttccgcatcatcgaccacaggccgggcatctcctcgcgcgacggcgcggagccagagtcggtgacggggcgggtggagatgcggggcgtggacttcgcgtacccgtcgcggccggacgtccccatcctgcgcggcttctcgctgagcgtgcccgccgggaagaccatcgcgctggtgggcagctccggctccgggaagagcacggtggtgtcgctcatcgagagattctacgaccccagcgcaggtatacctagtactgttactacttttagcgcattaatctgaggatgtccagttcgcttgcttgccaatcgccattgccatcgcaacaacaatacttcgccaactgccattgctgggtagattagtacagtagcagttagaagaagcctccactgtacattgcattgccaaacaaaagtgaattgtgcagtaactctgtaccaccacattgacatggaaatgaagtgaatgcttggagcatgcagagctggccggcctcatgggctgctgctacctgctagctagccaaccagaaccagccatcctctttcttgcttttctttttactttctttggtcgtggctgtttgtggtcatacatacattcacgcagagcagaagagctagctaagctaggtgggtgtgcctgcaacgcgggacaaagaaaactatttgttgcctggcaagatgctactgttgcctagcacatgcctgccattgaccgactgctcagtgagaagtggttcagttgtgctgttgacagtatagatagatatatatagtagccctgtagatttttttttcagacaaaaaaagaagaagaacgagatgaagtctgcaattcggttttggcagggcaaatcctgctggacgggcacgacctcaggtcgctggagctgcggtggctgcggcggcagatcgggctggtgagccaggagccggcgctgttcgcgacgagcatcagggagaacctgctgctggggcgggacagccagagcgcgacgctggcggagatggaggaggcggccagggtggccaacgcccactccttcatcatcaaactccccgacggctacgacacgcaggtccgtcccgtatagctagctcactagctgcactgccacttctctcgcttgctcccccaccgttgctgcctgttgctctccaatccacttgtcggtgtctggaccacacgtgctgcttgcctagctgctccacatctgctttccctgtccaaccttatgcaactcactctaatactatatcaaatacatttctagagtttaaagcttatcttagaataaatgcatctttagctacgagacaacctaacttcagttgttgttgttgttttttttactttctctcttctcacaaatactatgattacgtctttacagcgatcttttttattccaaacctaaaaatgcatgcactcactctaaaagcgcaaagggagcatctttttttcccccatcatctgcacgcagccttttcttttcctcatgtcacgaagggactgaaggtgtgtatgcagcgtcaagtcatccatccgttccactccactcactcatgcgtcgcgcactctgcgctcgtgcctgcccggggctaaagctttagtagctagcctcagatcagatactgttcgtgtttgttaggccgcggcagctgcacatgagctcatgacagccggcagcaccaccaccaacgccatggaagaggggtcggggtccatcacatagacataatgcctgttgtagactaggacgggagggcaattgttaggcgcctgttgccatcgcatttgctgctgtgggttgccaacaagtaacatgccaggatgctttgctatcacgcacaggacaggagaggtcctttttctcgacacaagctctacagcctctactaaactagcacttgctgatgagtgcagaggatgaatggacgatgaacatctagagtgagagagaaaaaaatgttaataataataaaaagtagtagcaggattaagaatcaacctggggtacgtaggaagaggtacaatccctaggaatctagagtatgagaagtatgggaggagttgggggagtgaaacggaacaaattccgagttggtattttgtcgggaatgtcaagttgatttttgatcctagtgcaagcaagaattatcaatcactcagactcagcctgtctgtgtctgtccaccccagctcttgctactctacttactactgtgctactagtgggtagggtaggtatcttacataaactgttattataaactgtcatctgagaaagagagccagtcaaacccatgctgctgcttattttaatcactgtcaaatggcaggcaggcaggcagtctggttagttaataacatctgggaagggtttaatcaaaccaaatcaaatcagacgaaatctagaggccacatgggatggggccatatgtactgtactagcataactagcggctagattttattagaacacggactcacactcccataactataactgacttgatcatgattccttgccaagcaatgctcgcatgcccatgcatgcatcatccctggtcaaactcaaacactctccaccgtcagggaataagacttattattttattaacaattcaatttttatttattaattacgtctggacgaggagtactggtttatttgatgagagacatggcagtccaagtcaaactcgtttgtctgaccatggcggtgatggccggtgcaggttggggagcgcggcctgcagctctccggtgggcagaagcagcgcatcgccatcgcccgcgccatgctcaagaaccccgccatcctgctgctggacgaggccaccagcgcgctggactccgagtctgagaagctcgtgcaggaggcgctggaccgcttcatgatggggcgcaccacccttggtgatcgcgcaacaggctgtccaccatccgcaaaggccgacgtggtggccgtgctgcagggcggcgccgtctccgagatgagcgcgcacgacgagctgatggccaagggcgagaacggcacctacgccaagctcatccgcatgcaggagcaggcgcacgaggcggcgctcgtcaacgcccgccgcagcagcgccaggccctccagcgcccgcaactccgtcagctcgcccatcatgacgcgcaactcctcctacggccgctccccctactcccgccgcctctccgacttctccacctccgacttcaccctctccatccacgacccgcaccaccaccaccggaccatggcggacaagcagctggcgttccgcgccggcgccagctccttcctgcgcctcgccaggatgaactcgcccgagtgggcctacgcgctcgccggctccatcggctccatggtctgcggctccttcagcgccatcttcgcctacatcctcagcgccgtgctcagcgtctactacgcgccggacccgcggtacatgaagcgcgagatcgcaaaatactgttacctgctcatcggcatgtcctccgcggcgctgctgttcaacacggtgcagcacgtgttctgggacacggtgggcgagaacttgaccaagcgggtgcgcgagaagatgttcgccgccgtgttccgcaacgagatcgcctggttcgacgcggacgagaacgccagcgcgcgcgtgaccgccaggctagcgctggacgcccagaacgtgcgctccgccatcggggaccgcatctccgtcatcgtccagaactcggcgctgatgctggtggcctgcaccgcggggttcgtcctccagtggcgcctcgcgctcgtgctcctcgccgtgttcccgctcgtcgtgggcgccaccgtgctgcagaagatgttcatgaagggcttctcgggggacctggaggccgcgcacgccagggccacgcagatcgcgggcgaggccgtggccaacctgcgcaccgtggccgcgttcaacgcggagcgcaagatcacggggctgttcgaggccaacctgcgcggcccgctccggcgctgcttctggaaggggcagatcgccggcagcggctacggcgtggcgcagttcctgctgtacgcgtcctacgcgctggggctgtggtacgcggcgtggctggtgaagcacggcgtgtccgacttctcgcgcaccatccgcgtgttcatggtgctgatggtgtccgcgaacggcgccgccgagacgctgacgctggcgccggacttcatcaaaggcgggcgcgcgatgcggtcggtgttcgagacaatcgaccgcaagacggaggtggagccccacgacgtggacgcggcgccggtgccggacggcccaggggcgaaggtggaacttaagcacgtggactttttgtacccgtcgcggccggacatccaagtgttccgcgacctgagcctccgtgcgcgcgccggaaaaacgttggcgctggtggggccgagcgggtccggcaagagctcggtcctggctctggtgcagcggttctacaagcccacgtccgggcgcgtgctcttggacggcaaggacgtgcgcaagtacaacctgcgggcgctgcggcgcgtggtggcggtggtaccgcaggagccgttcctgttcgcggcgagcatccacgagaacatcgcgtacgggcgcgagggcgcgacggaggcggaggtggtggaggcggcggcgcaggcgaacgcgcaccggttcatcgcggcgctgccggaggggtaccggacgcaggtgggcgagcgcggggtgcagctgtcgggggggcagcggcagcggatcgcgatcgcgcgcgcgctggtgaagcaggcggccatcgtgctgctggacgaggcgaccagcgcgctggacgccgagtcggagcggtgcgtgcaggaggcgctggagcgcgcggggtccgggcgcaccaccatcgtggtggcgcaccggctggccacggtgcgcggcgcgcacaccatcgcggtcatcgacgacggcaaggtggcggagcaggggtcgcactcgcacctgctcaagcaccatcccgacgggtgctacgcgcggatgctgcagcttgcagcggctgacgggcgcggcggccgggcccgggccgtcgtcctcgtgcaacggggccgcgtaggacggaatggatggatggatgggtttggttcctcgagagattgatgggtgaggaagctgaagctccggatcaaatggtggtactccatgatcgcaacaatgaggggaaaaaaggaaaggagaaaatacggtggttcatatgattgtacaatttgacgatctgtttgagtcggggttttaggatgatgtaaaccttcactcgccttttttttactcttgtttctcatccgcatcagtatcatctatctacatacagtgtcagagatgggaactgatcccgcatcatcatctacctcccaaggcaccccagattgtattaatgtacttagttagcctgttttatatatacttataagtaccaaatagcagaattttactccttatctgcagtagcacgaaagaaaaaaaaaaaaagctaaacct

SEQ ID NO: 14 - DNA, 5845 bp, Zea mays
  modified_base (4245)..(4344): a, c, g or t
gtcctcctctggaaaaccacctcttctcctagacttcttccgttgcatcttctcttctgttctccacgacgcagttcggctgccagacaaaaaccctaaacgccgccgccgccgccgctcttcctcgagatgggaggcaagtgcccgcaccgcaaggtaaagaagcgccgcctatcccacaagacggctcgccgcggcaagttcctcctgaaggccgacgatgccgtctatgacgagctcgtcaagctggccgaccagggcaaggacgctgaagccaaggagctccccgtcgatgaggacctcccaggcctgggacagttctactgcctccactgcgagtatgcgcttcccctctcctctctttccctcccttccattccaccggtatgctatttcttatcctctgccaatacaggtatatgcccgtacgttgctagttgtaaaaaaaaacgtttttctcaattcagcattagtggagcatgtgttttaattcattcgtggtacaaaaaacacctgactagcgaatgataatcctgtttatctttgcaaaaactaatttgaatttccatgtgtgcatgaattaaaacttaccgtactgtcacacaataagatcttaaaatactggatactcttagtacacaaatgatccaaaagaaagcacaattccagcattccagtctttcgctatttcatcatcctcatgtgccaacaattattgtttgtttcccttcctggacacacagaaaaatgcaatgggatccagcaccgtaattaggccgcaacaaagggtgaaattgtatagggaattttcaagcacccactctaggagtctagggccatcaatttggtttacttcgtcaaagaacggatgaaattgaataagtttcatatctatggtgtaaacatctactataccgatatgtagaaactgagaactattcctgctgcaccgactgtaaagctggcatagctgaatatgtagagcttcttgttgatggggatgtctgcaagagcttaccgtttggtatttgcattgagaatggactgtgatttctttctttgacgtgcttcacaatatccaattctctaattgggtggtgatgtgtgtgtgtgggggggggggacccctaataaatctgctaaaacctccttgaagtctctatttcacccgcctttttttcctcccttatttctaaacttttagtactattttaccttcccttagttatgtacatctaataaataccacaatagtggaagcaattactgtctgtttccacaaaaatagagcattgaggaaccaatttgtccatcatttgttgccacttctaggaagtcggttggcaggcacatatatctaagctcttaatatatcatatcaaatagtaaatatctctaatcaacaaaaagggaaaccccaattataaaatttatcccgtaaatataatttatttttctagctgtgctcagagagtaaaagcaactccaaaagaagatgtaaaccaaccttaaaacagatattaggaagaaaatgattttccttgctccaatagctccctcatcgcccccaaaaaaattgcatcccacatccgcctcctcccctcccgcatattttcgcgtgatccctttctccccaatctgttcggcgtctgcgtgcatgcaggctcctgtgttttttattttgccgcgcgctcatctgtggccgtgcagaccatcaatgagttggtacagctttcctgcagcatgtggcgtcgtaggatcatcatgctggatcagcataaaggcccatatacccgcccatacccaggagcaaagccagttttcagttttttagttgtttaaggaaactgttggagtacacactataatttactcttaatttttttagaatgcctccaataatcatttttttggggaaactttttatcattcttttggagttagctggtgggtcttacaggtctgagcttgctggccaatggcagcattgatgaaggtaatgttactgctaaataggcaaaatccaaatctaagagcccaaagcactggcaattcaatgaccagacttgacaaggggcagatcatgacatggcgagatgatggacataagatgaactttgcaggcttcatctttagaccatgatatttatctcatgtttatccacttatagataaaccctgtttgtggagccagaagatacacctatatgaaatttgacctgttggtatgagaaccaggaaaaagcgaccgccctccattggtacaagaatttcagaacagagagtcaagagacatctgacagtttgcaatcacaccatgcaaccattgtagcatggaatggccaaagactaattatgaaccaaggaatgatggtgtcaaacttggtccagtgcataccatctaacaaaacctggtgcagtaatagccattatcatctcacaaggacctcttttctctctttacaaaatctaataaggattctacaaattggatacattgtaatattgaataacgcaagcagaaaggtgtctgtgtgtctctgcaatttgtctgtcacaatgatacaacaacaacaaagcctttttgtcccaagcacgttggggtaggctagagatgaaaccccgtatgaaaacctcagagctcaacccccaaagaacagaaaagggaaacaaaggcaaaggagaaccgaaaacaacgaaacggggaaacacataaaagagataaaacccacaaggaccagcaagatctaaaatggacacaagaaaaggtcaaacgattaaggaggaaaagcgaaactatcaatcagggttctggcacgtgaattgcacacttccaccccttcctatctgcggcgagctctttatcaatattccactccttcaggtctctcttcacagcctctgtccacgtcaaagttggtcggcctctacctctcttcacattttcgggacgcctaattattccgatatgcactggtgcctcttcaggtcttcgttggatatgtccaaaccatctcaagcgatgttgcataagcttctcctcaattggcgccactcctactctctctcgtatatcatcattccggactcgatctctccttgtgtggccacatatccagcgcaacatacgcatctctgccacacttagttgttggacatgtcgtcttttagtgggccaacattctgctccatacaacatagccggccggattgttgtcctgtagaatttgccttttagtttgcgtggcacccggtggtcgcataagacacccgcagcttgtcgccacttcaaccatccggctttaattctatgactgacatcctcatcgatgtctccctccttttgaagcattgatcctaagtaacgaaaagtgtctttcttgggtaccacttgcccatcaagactaacatcgccatcctcataccccatggcactgaaatcacacttcatatactctgttttagacctactaagcctaaaaccttttgcttccagcgtctgcctccacaattccaacttttgagaaacaccactcctactctcctcaataagtaccacatcatcggcaaaaagcatacaccacggtaaaactccttgtatgtcccttgtgacctcatctatcaccaaagcgaaaagataagggctcaaagccgacccttgatgtagtcctatgttaattggaaagtcatcggtgtctccatcacttgttcggacacttgtcacaacattagtgtacatatctttaataagattaatgtactttgttgctactttgtgtttttccaaggcccaccacattacactcctaggtactttgtcataagccttctccaagtctatgaagaccatatgcaggtctttcttttgttctctgaatctctccataagttgtcttagtaagaaaatggcctccatagttgatctcccgggcatgaaaccaaactgattttgggtcacgctcgtcatctttcgcaggcggtgttcaatgactctctcccatagcttcattgtatggctcatgagcttaattccacggtaattagtacaactttgaacatctcctttgttcttgaagattggtactaatgtactccgccgccactcgtcgggcattctgtttgcccgaaagatggtgttgaaaagcttagtcagccatactatcgctatgtctccaagacatctccatacctcgatagggataccatcagggcccatcgcctttcctaccttcatcctttttagcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntctttttagtggtagaagatcatacgagctgttcctgaactctcttgtattgtcttatcagcaaacgatagggcaatttcagtgagttacggttggttcatttgatgaattagttgtatggaagtgcacagtttataagatgctgctagtttttgtgttgatattaataaactgagtgtttcagttgagttgtctggtatagagtaaaatggtgaaatttggttgatgtttttgtgatttaatgcttcctggaatttatgatagtagatgccttgaattgatttagagggattagcaaaactgtggctacactttaaggtgtgattgctggcgttacaatgcttgtgatgagaactgcacttatcttgtaaatgtggtagctatattctaatctcctactgctaatcgatatatatctggtattggctttggctgcagtcgctactttgcaagtgaaagcgtgaaggatgatcactaccgctcaaagcgccacaagaaaaggtgatccattctgtttgctctaagtctaagtaatatcacggtaatcatcattcatagataacattaaagaaaaaactatcctgaacacatttgatttatgctaccagtttgattggcaaacatgtaatgacctgcacttataagctgtttggtttgaggaatgatctagtccatcgtcttctcactcctcacttttttgtttggtttgtggaatggattgagttgatccatcatcgcctcattccttatagttatttagttagtactaatatgaggaatgaggtcatccaaccaaatttgaggaatggatccatgatgcatcactacattttggatgaaatgattcctcaaaccaaacacctccttagagagcatctccaacaagaagttctatatttgagtcctaaaaaattaaaataggacctgattttaagtgttcaggaccacaaaaacatttgcagctccaacggttgagtcctataaccagttgataaaacttggagggaccttgttgtgtggttctatatgacaccataactaaattagtgttgatcatgcgatagtaactaaattagtgttggaccttgttgtgtggttcgataactaaattaccagtatgacctttcccgtcggcagaccatgtgagaaatgtggtataggacaggttctggtacacgtgagtggaataattccgacctgctgtggcgtgtgcctgtctctaatcacaggcttcggatgattgtagggttaaggtaatgtctggaccggccccgcacacgcagcttgatgcagaactcgctgcagggatgggaaagcctgacaacggcctgaagctcatgtccatgtgaaggaacctcttgctggtactggtacttgttccgtgcctttgttgctgccgtacaattgaagctgctttgcgcagtaagtaatagcaagagttatagcagacggtttttttttgttgtacgcaagactttcgaagtgggtgtaagttgtttta

SEQ ID NO: 15 - RNA, 20 nt, Artificial Sequence
  Description of Artificial Sequence Synthetic oligonucleotide
ggcaaucugggcauccucgu

SEQ ID NO: 16 - RNA, 20 nt, Artificial Sequence
  Description of Artificial Sequence Synthetic oligonucleotide
ccgccgcucuuccucgagau

SEQ ID NO: 17 - DNA, 30 bp, Zea mays
agcagcctcgccgttagacccgtaggagca

SEQ ID NO: 18 - DNA, 50 bp, Zea mays
ccctccgttcacgggcgtggcgttccatttcttcgcggcggatagggtgt

SEQ ID NO: 19 - DNA, 80 bp, Zea mays
acgaggatgcccagattgccgctccgacgaccctccgttcacgggcgtggcgttccatttcttcgcggcggatagggtgt

SEQ ID NO: 20 - DNA, 30 bp, Zea mays
atctcgaggaagagcggcggcggcggcggc

SEQ ID NO: 21 - DNA, 50 bp, Zea mays
gacgcacacaagctggggcggcaggaggaggagggatagcgtacctgccc

SEQ ID NO: 22 - DNA, 80 bp, Zea mays
gacgcacacaagctggggcggcaggaggaggagggatagcgtacctgcccatctcgaggaagagcggcggcggcggcggc

SEQ ID NO: 23 - DNA, 13308 bp, Zea mays
  modified_base (4775)..(4874): a, c, g or t
  modified_base (6376)..(6699): a, c, g or t
gctctgccactctgctgaggtggggggagaggagctccccctccctcctctcccctcctcgccatgtctagcagcgacccggaggagatcagggcgcgcgtcgtcgttctcggttcgccccatgccgacggcggcgacgagtgggcccggcccgagctcgaggccttccatctgccgtctcccgcccaccagcctcctggcttcctagccgggcaaccggaagcagcagagcaacccacgctccctgctcctgctggccgcagcagcagcagcagcaacacgcctactacatctgccggtggcggcgctgctcctcctcctccttcttcgcctccccctccgccggcttctctggagaccgagcagccgcccaatgccaggccagcctccgccggcgccaatgacagcaagaagcccaccccgcccgccgccctgcgcgacctcttccgcttcgccgacggcctcgactgcgcgctcatgctcatcggcaccctcggcgcgctcgtccacgggtgctcgctccccgtcttcctccgcttcttcgccgacctcgtcgactccttcggctcccacgccgacgacccggacaccatggtccgcctcgtcgtcaagtacgccttctacttcctcgtcgtcggagcggcaatctgggcatcctcgtgggaggcaagtgcccgcaccgcaaggtaaagaagcgccgcctatcccacaagacggctcgccgcggcaagttcctcctgaaggccgacgatgccgtctatgacgagctcgtcaagctggccgaccagggcaaggacgctgaagccaaggagctccccgtcgatgaggacctcccaggcctgggacagttctactgcctccactgcgagtatgcgcttcccctctcctctctttccctcccttccattccaccggtatgctatttcttatcctctgccaatacaggtatatgcccgtacgttgctagttgtaaaaaaaaacgtttttctcaattcagcattagtggagcatgtgttttaattcattcgtggtacaaaaaacacctgactagcgaatgataatcctgtttatctttgcaaaaactaatttgaatttccatgtgtgcatgaattaaaacttaccgtactgtcacacaataagatcttaaaatactggatactcttagtacacaaatgatccaaaagaaagcacaattccagcattccagtctttcgctatttcatcatcctcatgtgccaacaattattgtttgtttcccttcctggacacacagaaaaatgcaatgggatccagcaccgtaattaggccgcaacaaagggtgaaattgtatagggaattttcaagcacccactctaggagtctagggccatcaatttggtttacttcgtcaaagaacggatgaaattgaataagtttcatatctatggtgtaaacatctactataccgatatgtagaaactgagaactattcctgctgcaccgactgtaaagctggcatagctgaatatgtagagcttcttgttgatggggatgtctgcaagagcttaccgtttggtatttgcattgagaatggactgtgatttctttctttgacgtgcttcacaatatccaattctctaattgggtggtgatgtgtgtgtgtgggggggggggacccctaataaatctgctaaaacctccttgaagtctctatttcacccgcctttttttcctcccttatttctaaacttttagtactattttaccttcccttagttatgtacatctaataaataccacaatagtggaagcaattactgtctgtttccacaaaaatagagcattgaggaaccaatttgtccatcatttgttgccacttctaggaagtcggttggcaggcacatatatctaagctcttaatatatcatatcaaatagtaaatatctctaatcaacaaaaagggaaaccccaattataaaatttatcccgtaaatataatttatttttctagctgtgctcagagagtaaaagcaactccaaaagaagatgtaaaccaaccttaaaacagatattaggaagaaaatgattttccttgctccaatagctccctcatcgcccccaaaaaaattgcatcccacatccgcctcctcccctcccgcatattttcgcgtgatccctttctccccaatctgttcggcgtctgcgtgcatgcaggctcctgtgttttttattttgccgcgcgctcatctgtggccgtgcagaccatcaatgagttggtacagctttcctgcagcatgtggcgtcgtaggatcatcatgctggatcagcataaaggcccatatacccgcccatacccaggagcaaagccagttttcagttttttagttgtttaaggaaactgttggagtacacactataatttactcttaatttttttagaatgcctccaataatcatttttttggggaaactttttatcattcttttggagttagctggtgggtcttacaggtctgagcttgctggccaatggcagcattgatgaaggtaatgttactgctaaataggcaaaatccaaatctaagagcccaaagcactggcaattcaatgaccagacttgacaaggggcagatcatgacatggcgagatgatggacataagatgaactttgcaggcttcatctttagaccatgatatttatctcatgtttatccacttatagataaaccctgtttgtggagccagaagatacacctatatgaaatttgacctgttggtatgagaaccaggaaaaagcgaccgccctccattggtacaagaatttcagaacagagagtcaagagacatctgacagtttgcaatcacaccatgcaaccattgtagcatggaatggccaaagactaattatgaaccaaggaatgatggtgtcaaacttggtccagtgcataccatctaacaaaacctggtgcagtaatagccattatcatctcacaaggacctcttttctctctttacaaaatctaataaggattctacaaattggatacattgtaatattgaataacgcaagcagaaaggtgtctgtgtgtctctgcaatttgtctgtcacaatgatacaacaacaacaaagcctttttgtcccaagcacgttggggtaggctagagatgaaaccccgtatgaaaacctcagagctcaacccccaaagaacagaaaagggaaacaaaggcaaaggagaaccgaaaacaacgaaacggggaaacacataaaagagataaaacccacaaggaccagcaagatctaaaatggacacaagaaaaggtcaaacgattaaggaggaaaagcgaaactatcaatcagggttctggcacgtgaattgcacacttccaccccttcctatctgcggcgagctctttatcaatattccactccttcaggtctctcttcacagcctctgtccacgtcaaagttggtcggcctctacctctcttcacattttcgggacgcctaattattccgatatgcactggtgcctcttcaggtcttcgttggatatgtccaaaccatctcaagcgatgttgcataagcttctcctcaattggcgccactcctactctctctcgtatatcatcattccggactcgatctctccttgtgtggccacatatccagcgcaacatacgcatctctgccacacttagttgttggacatgtcgtcttttagtgggccaacattctgctccatacaacatagccggccggattgttgtcctgtagaatttgccttttagtttgcgtggcacccggtggtcgcataagacacccgcagcttgtcgccacttcaaccatccggctttaattctatgactgacatcctcatcgatgtctccctccttttgaagcattgatcctaagtaacgaaaagtgtctttcttgggtaccacttgcccatcaagactaacatcgccatcctcataccccatggcactgaaatcacacttcatatactctgttttagacctactaagcctaaaaccttttgcttccagcgtctgcctccacaattccaacttttgagaaacaccactcctactctcctcaataagtaccacatcatcggcaaaaagcatacaccacggtaaaactccttgtatgtcccttgtgacctcatctatcaccaaagcgaaaagataagggctcaaagccgacccttgatgtagtcctatgttaattggaaagtcatcggtgtctccatcacttgttcggacacttgtcacaacattagtgtacatatctttaataagattaatgtactttgttgctactttgtgtttttccaaggcccaccacattacactcctaggtactttgtcataagccttctccaagtctatgaagaccatatgcaggtctttcttttgttctctgaatctctccataagttgtcttagtaagaaaatggcctccatagttgatctcccgggcatgaaaccaaactgattttgggtcacgctcgtcatctttcgcaggcggtgttcaatgactctctcccatagcttcattgtatggctcatgagcttaattccacggtaattagtacaactttgaacatctcctttgttcttgaagattggtactaatgtactccgccgccactcgtcgggcattctgtttgcccgaaagatggtgttgaaaagcttagtcagccatactatcgctatgtctccaagacatctccatacctcgatagggataccatcagggcccatcgcctttcctaccttcatcctttttagcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntctttttagtggtagaagatcatacgagctgttcctgaactctcttgtattgtcttatcagcaaacgatagggcaatttcagtgagttacggttggttcatttgatgaattagttgtatggaagtgcacagtttataagatgctgctagtttttgtgttgatattaataaactgagtgtttcagttgagttgtctggtatagagtaaaatggtgaaatttggttgatgtttttgtgatttaatgcttcctggaatttatgatagtagatgccttgaattgatttagagggattagcaaaactgtggctacactttaaggtgtgattgctggcgttacaatgcttgtgatgagaactgcacttatcttgtaaatgtggtagctatattctaatctcctactgctaatcgatatatatctggtattggctttggctgcagtcgctactttgcaagtgaaagcgtgaaggatgatcactaccgctcaaagcgccacaagaaaaggtgatccattctgtttgctctaagtctaagtaatatcacggtaatcatcattcatagataacattaaagaaaaaactatcctgaacacatttgatttatgctaccagtttgattggcaaacatgtaatgacctgcacttataagctgtttggtttgaggaatgatctagtccatcgtcttctcactcctcacttttttgtttggtttgtggaatggattgagttgatccatcatcgcctcattccttatagttatttagttagtactaatatgaggaatgaggtcatccaaccaaatttgaggaatggatccatgatgcatcactacattttggatgaaatgattcctcaaaccaaacacctccttagagagcatctccaacaagaagttctatatttgagtcctaaaaaattaaaataggacctgattttaagtgttcaggaccacaaaaacatttgcagctccaacggttgagtcctataaccagttgataaaacttggagggaccttgttgtgtggttctatatgacaccataactaaattagtgttgatcatgcgatagtaactaaattagtgttggaccttgttgtgtggttcgataactaaattaccagtatgacctttcccgtcggcagaccatgtgagaaatgtggtataggacaggttctggtacacgtgagtggaataattccgacctgctgtggcgtgtgcctgtctctaatcacaggcttcggatgattgtagggttaaggtaatgtctggaccggccccgcacacgcagcttgatgcagaactcgctgcagggatgggaaagcctgacaacggcctgaagctcatgtccatgtgaaggaacctcttgctggtactggtacttgttccgtgcctttgttgctgccgtacaattgaagctgctttgcgcagtaagtaatagcaagagttatagcagacggtttttttttgttgtacgcaagactttcgaagtgggtgtaagttgttttannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaggtttagctttttttttttttctttcgtgctactgcagataaggagtaaaattctgctatttggtacttataagtatatataaaacaggctaactaagtacattaatacaatctggggtgccttgggaggtagatgatgatgcgggatcagttcccatctctgacactgtatgtagatagatgatactgatgcggatgagaaacaagagtaaaaaaaaggcgagtgaaggtttacatcatcctaaaaccccgactcaaacagatcgtcaaattgtacaatcatatgaaccaccgtattttctcctttccttttttcccctcattgttgcgatcatggagtaccaccatttgatccggagcttcagcttcctcacccatcaatctctcgaggaaccaaacccatccatccatccattccgtcctacgcggccccgttgcacgaggacgacggcccgggcccggccgccgcgcccgtcagccgctgcaagctgcagcatccgcgcgtagcacccgtcgggatggtgcttgagcaggtgcgagtgcgacccctgctccgccaccttgccgtcgtcgatgaccgcgatggtgtgcgcgccgcgcaccgtggccagccggtgcgccaccacgatggtggtgcgcccggaccccgcgcgctccagcgcctcctgcacgcaccgctccgactcggcgtccagcgcgctggtcgcctcgtccagcagcacgatggccgcctgcttcaccagcgcgcgcgcgatcgcgatccgctgccgctgcccccccgacagctgcaccccgcgctcgcccacctgcgtccggtacccctccggcagcgccgcgatgaaccggtgcgcgttcgcctgcgccgccgcctccaccacctccgcctccgtcgcgccctcgcgcccgtacgcgatgttctcgtggatgctcgccgcgaacaggaacggctcctgcggtaccaccgccaccacgcgccgcagcgcccgcaggttgtacttgcgcacgtccttgccgtccaagagcacgcgcccggacgtgggcttgtagaaccgctgcaccagagccaggaccgagctcttgccggacccgctcggccccaccagcgccaacgtttttccggcgcgcgcacggaggctcaggtcgcggaacacttggatgtccggccgcgacgggtacaaaaagtccacgtgcttaagttccaccttcgcccctgggccgtccggcaccggcgccgcgtccacgtcgtggggctccacctccgtcttgcggtcgattgtctcgaacaccgaccgcatcgcgcgcccgcctttgatgaagtccggcgccagcgtcagcgtctcggcggcgccgttcgcggacaccatcagcaccatgaacacgcggatggtgcgcgagaagtcggacacgccgtgcttcaccagccacgccgcgtaccacagccccagcgcgtaggacgcgtacagcaggaactgcgccacgccgtagccgctgccggcgatctgccccttccagaagcagcgccggagcgggccgcgcaggttggcctcgaacagccccgtgatcttgcgctccgcgttgaacgcggccacggtgcgcaggttggccacggcctcgcccgcgatctgcgtggccctggcgtgcgcggcctccaggtcccccgagaagcccttcatgaacatcttctgcagcacggtggcgcccacgacgagcgggaacacggcgaggagcacgagcgcgaggcgccactggaggacgaaccccgcggtgcaggccaccagcatcagcgccgagttctggacgatgacggagatgcggtccccgatggcggagcgcacgttctgggcgtccagcgctagcctggcggtcacgcgcgcgctggcgttctcgtccgcgtcgaaccaggcgatctcgttgcggaacacggcggcgaacatcttctcgcgcacccgcttggtcaagttctcgcccaccgtgtcccagaacacgtgctgcaccgtgttgaacagcagcgccgcggaggacatgccgatgagcaggtaacagtattttgcgatctcgcgcttcatgtaccgcgggtccggcgcgtagtagacgctgagcacggcgctgaggatgtaggcgaagatggcgctgaaggagccgcagaccatggagccgatggagccggcgagcgcgtaggcccactcgggcgagttcatcctggcgaggcgcaggaaggagctggcgccggcgcggaacgccagctgcttgtccgccatggtccggtggtggtggtgcgggtcgtggatggagagggtgaagtcggaggtggagaagtcggagaggcggcgggagtagggggagcggccgtaggaggagttgcgcgtcatgatgggcgagctgacggagttgcgggcgctggagggcctggcgctgctgcggcgggcgttgacgagcgccgcctcgtgcgcctgctcctgcatgcggatgagcttggcgtaggtgccgttctcgcccttggccatcagctcgtcgtgcgcgctcatctcggagacggcgccgccctgcagcacggccaccacgtcggcctttgcggatggtggacagcctgttgcgcgatcaccaagggtggtgcgccccatcatgaagcggtccagcgcctcctgcacgagcttctcagactcggagtccagcgcgctggtggcctcgtccagcagcaggatggcggggttcttgagcatggcgcgggcgatggcgatgcgctgcttctgcccaccggagagctgcaggccgcgctccccaacctgcaccggccatcaccgccatggtcagacaaacgagtttgacttggactgccatgtctctcatcaaataaaccagtactcctcgtccagacgtaattaataaataaaaattgaattgttaataaaataataagtcttattccctgacggtggagagtgtttgagtttgaccagggatgatgcatgcatgggcatgcgagcattgcttggcaaggaatcatgatcaagtcagttatagttatgggagtgtgagtccgtgttctaataaaatctagccgctagttatgctagtacagtacatatggccccatcccatgtggcctctagatttcgtctgatttgatttggtttgattaaacccttcccagatgttattaactaaccagactgcctgcctgcctgccatttgacagtgattaaaataagcagcagcatgggtttgactggctctctttctcagatgacagtttataataacagtttatgtaagatacctaccctacccactagtagcacagtagtaagtagagtagcaagagctggggtggacagacacagacaggctgagtctgagtgattgataattcttgcttgcactaggatcaaaaatcaacttgacattcccgacaaaataccaactcggaatttgttccgtttcactcccccaactcctcccatacttctcatactctagattcctagggattgtacctcttcctacgtaccccaggttgattcttaatcctgctactactttttattattattaacatttttttctctctcactctagatgttcatcgtccattcatcctctgcactcatcagcaagtgctagtttagtagaggctgtagagcttgtgtcgagaaaaaggacctctcctgtcctgtgcgtgatagcaaagcatcctggcatgttacttgttggcaacccacagcagcaaatgcgatggcaacaggcgcctaacaattgccctcccgtcctagtctacaacaggcattatgtctatgtgatggaccccgacccctcttccatggcgttggtggtggtgctgccggctgtcatgagctcatgtgcagctgccgcggcctaacaaacacgaacagtatctgatctgaggctagctactaaagctttagccccgggcaggcacgagcgcagagtgcgcgacgcatgagtgagtggagtggaacggatggatgacttgacgctgcatacacaccttcagtcccttcgtgacatgaggaaaagaaaaggctgcgtgcagatgatgggggaaaaaaagatgctccctttgcgcttttagagtgagtgcatgcatttttaggtttggaataaaaaagatcgctgtaaagacgtaatcatagtatttgtgagaagagagaaagtaaaaaaaacaacaacaacaactgaagttaggttgtctcgtagctaaagatgcatttattctaagataagctttaaactctagaaatgtatttgatatagtattagagtgagttgcataaggttggacagggaaagcagatgtggagcagctaggcaagcagcacgtgtggtccagacaccgacaagtggattggagagcaacaggcagcaacggtgggggagcaagcgagagaagtggcagtgcagctagtgagctagctatacgggacggacctgcgtgtcgtagccgtcggggagtttgatgatgaaggagtgggcgttggccaccctggccgcctcctccatctccgccagcgtcgcgctctggctgtcccgccccagcagcaggttctccctgatgctcgtcgcgaacagcgccggctcctggctcaccagcccgatctgccgccgcagccaccgcagctccagcgacctgaggtcgtgcccgtccagcaggatttgccctgccaaaaccgaattgcagacttcatctcgttcttcttctttttttgtctgaaaaaaaaatctacagggctactatatatatctatctatactgtcaacagcacaactgaaccacttctcactgagcagtcggtcaatggcaggcatgtgctaggcaacagtagcatcttgccaggcaacaaatagttttctttgtcccgcgttgcaggcacacccacctagcttagctagctcttctgctctgcgtgaatgtatgtatgaccacaaacagccacgaccaaagaaagtaaaaagaaaagcaagaaagaggatggctggttctggttggctagctagcaggtagcagcagcccatgaggccggccagctctgcatgctccaagcattcacttcatttccatgtcaatgtggtggtacagagttactgcacaattcacttttgtttggcaatgcaatgtacagtggaggcttcttctaactgctactgtactaatctacccagcaatggcagttggcgaagtattgttgttgcgatggcaatggcgattggcaagcaagcgaactggacatcctcagattaatgcgctaaaagtagtaacagtactaggtatacctgcgctggggtcgtagaatctctcgatgagcgacaccaccgtgctcttcccggagccggagctgcccaccagcgcgatggtcttcccggcgggcacgctcagcgagaagccgcgcaggatggggacgtccggccgcgacgggtacgcgaagtccacgccccgcatctccacccgccccgtcaccgactctggctccgcgccgtcgcgcgaggagatgcccggcctgtggtcgatgatgcggaagatcttggcagccgccacacgcgccttggcgaacgcggccatgctcggcgccgactgccgaggcctgcacgcgatccacagataccgtcagcagcaatgacgacgggggcggccgaggacagcagaaggagagagtgcgtggtgtggtgggccttacagtccgccgatcatgacggagaacatggtggcgatggcgagcccgccgttggtgtgctgggcgcgcacgaggtggccgccgtaccagagcaggagcccgtagcagcagaagacggtgaagtaggtgccgccgaggccgagccccttggcgaagccgctgcggtagccgatcctctgcgccacggccagcgccgccgagtaggcccgcatctcgcgctcctcgccaacgaacgcctgcacgatccgtatctgcgcgagcgcctgctccgcgatgccgctggcgcccgagagcgcgtcctggctgcgggacgagagcttggcgagcgcggcggcgctcagcccgccgatgacggcgatgagcggcaccacggccagcgtgaccagcgccagctgccacgcggccgtgaaccccacgacgaagccggccacgaaggtggccatgtagtggatgaggttgcccagtttctggctgatggcgtcctgcaccaccacggcgtccgcgttgatggcgtagatcacgtccgaggcccgcacgtcggtgtcgaagaaggacacgtcctgccgcagcgccgcgtccaggtaccgaatccgcatccgcgtcgactgccgctcgccggtccacatccagcaagagatctcttgcgagcggggcgaacgatttgaacatctcgtttgtagccacggcgattggctgaccgacgtgatttgtcatccaatccaaattgaccgccaattcgcgacgcacacaagctggggcggcaggaggaggagggatagcgtacctgcccatctcgaggaagagcggcggcggcggcggcgtttagggtttttgtctggcagccgaactgcgtcgtggagaacagaagagaagatgcaacggaagaagtctaggagaagaggtggttttccagaggaggac

SEQ ID NO: 24 - DNA, 3860 bp, Zea mays
gctccttgctgttctgctgactggtctcaccatctcatcccaccaccaccaccaccaccaccatctttaggataagatagcaaatatatggccatcatactcgtacgagcagcgtcgccggggctctccgccgccgacagcatcagccaccaggggactctccagtgctccaccctgctcaagacgaagaggccggcggcgcgccggtggatgccctgctcgctccttggcctccacccgtgggaggctggccgtccctcccccgccgtctactccagcctcgccgtcaacccggcgggagaggccgtcgtctcgtccgagcagaaggtctacgacgtcgtgctcaagcaggccgcattgctcaaacgccagctgcgcacgccggtcctcgacgccaggccccaggacatggacatgccacgcaacgggctcaaggaagcctacgaccgctgcggcgagatctgtgaggagtatgccaagacgttttacctcggtacgtacggtatatatatgggatccatcttcttctccaattccacaatctcatcgtttcagtctgcttccatcactcatgctactagttcgtcgcaggaactatgttgatgacagaggagcggcgccgcgccatatgggccatctatggtatctgtctgtctcaaatacaataatcaccatgcatgtatccctccaatgtatcagtaccattgctcatacctagctagtagcatgttacgtacggagtatcaatcagtaaatttcagaatggctactactggaactggatgcgctgtactagctagtatgtttccctacttaatatataacgtagatacgcacttctgacttaattatgctgctgtggcaaaaggattttttttttttactttaacagcagattccaacaataacagtgcaaaattgggctacttttcaagtaatggtaacaactagcagctcccagtggaaattaacaatattgaaaaaagaaacactgctgacttccatgaaggccattcgtgagtcaatggtgaataaaggtttaatgcgctttatgttgtatgtggaagcaccaaaataggctttcggtagcctaaattgctactaaatggcttgatcaatacttgaagaccatgtggaaagttataagaacatattctttatagttcaaactaccctttaggaatatgaatctcaagtattggcaattttaaacgaacacttcaggcacggtatctgacgggtattgtttctgcagtgtggtgtaggaggacagatgagcttgtagatgggccaaacgccaactacattacaccaacagctttggaccggtgggagaagagacttgaggatctgttcacgggacgtccttacgacatgcttgatgccgctctctctgataccatctcaaggttccccatagacattcaggtactgactgccttacgggctgctgtacctagcggattcattccactgatatgacacttttggactgcagttattgatatttctaaccagcattagattctctagctaggcctcactgtttttagtgcaggatgactagacctactgagttgacaagaagctagcagaaattgttttgtttactcaactgaatcttaagattttttcaacctttagtctcttctagcaatgcttttgtttggttcaatgaaccttgcgcatatcgtagtggtcctagtctagttatatggaccaggacccaggagaggtgtttgggtcaaagctgtttcaatgtaacttttttttaaataaaaaatgaactgtttctagagctcagccctcacagcaaaacttggagcgagaggcaataatttgaatattacatggccttggtaatgtgaattaattagttggtctatccttagttggtccgaccttttaagaaacaaaaggtaactgtatgaccagcgcagaagaaaataggatctagatgatgattgagagagaaacgtttcagggggaaaaaatccaatcaattaaaaattagacctgggaacattggcagatcgatctagagggtggacaaggtgggctgattttgatcctgctaaattatatagatagttttgatttatttgctacttttgattctcatacgttgtagaacttaaaatgtgaactcatttgtttattgattctcataaggttggaccaccttaactttaaatcctagatttgccactgggaagtgaccgagagaaaactatctgtgccacttagtggtttgataaactatgttgtgataccaagttaccaacgttttgaaatcaataaatgttgtggcagccattcagggacatgattgaagggatgaggagtgatcttaggaagacaaggtataacaacttcgacgagctctacatgtactgctactatgttgctggaactgtcgggttaatgagcgtacctgtgatgggcatcgcaaccgagtctaaagcaacaactgaaagcgtatacagtgctgccttggctctgggaattgcgaaccaactcacgaacatactccgggatgttggagaggagtaagtaacatatatattcttcctgcgacaggcacgaacatgcatgtgttcaatagcacagatgtgatgatatgactgtcaccatgtcttttagtgctagaagaggaaggatatatttaccacaagatgagcttgcacaggcagggctctctgatgaggacatcttcaaaggggtcgtcacgaaccggtggagaaacttcatgaagaggcagatcaagagggccaggatgttttttgaggaggcagagagaggggtaactgagctctcacaggctagcagatggccagtaagtccactcaacttcacatttcccacccagtatagcacagcatcctcacttccttttctttgttaccattgcaggtatgggcttccctgttgttgtacaggcagatcctggatgagatcgaagccaacgactacaacaacttcacgaagagggcgtatgttggtaaagggaagaagttgctagcacttcctgtggcatatggaaaatcgctactgctcccatgttcattgagaaatggccagacctagccaccagagaagctgcaatgcaaggttcaggttaggctagatagaaagttaaatggggcaacatcaggaggccttgatgaaaaacagacaacctggtgaattgttgttgggatcaggcacagaacagataagagccgcgcagccaacctagggcatgtttggtttcaattagttctaggactaaactttagtcctaggactaaactttagtccctatatgtttggttctagggactaaatagattctaaagtcattaaatacattgtccaaagactcaaatacccttagaatatactcatgatattagttatctataaaaaggtaagggcaacatgataattatgagcttttagtctcttttagcacctatgtgaaggactaaagactaaatcattttagtccatattttagtcctagtgtttggcaaaaaagggactaaaagggactaaaaactagagactaatctttagtccctctaaccaaacacccccctagatggatacggaacattcgcctcttattcggagcaatatatgtctctcaaggaaagagcccaacatgtatactgccttctttttctcatcccagatttgggggaaaaacaatgtaaatgccaatggtatcgtaggaagattactagaagtaaatgccaatgtaaaaacagatgagttggcatttacatgataggatggtgggatcatcagactgaaaatgataggggattgtgctcccctgcgactccaactattaaacaaggaatccgtcagcagta

SEQ ID NO: 25 - DNA, 248 bp, Zea mays
atgttgtggcagccattcagggacatgattgaagggatgaggagtgatcttaggaagacaaggtataacaacttcgacgagctctacatgtactgctactatgttgctggaactgtcgggttaatgagcgtacctgtgatgggcatcgcaaccgagtctaaagcaacaactgaaagcgtatacagtgctgccttggctctgggaattgcgaaccaactcacgaacatactccgggatgttggagagga

SEQ ID NO: 26 - DNA, 248 bp, Zea mays
tcctctccaacatcccggagtatgttcgtgagttggttcgcaattcccagagccaaggcagcactgtatacgctttcagttgttgctttagactcggttgcgatgcccatcacaggtacgctcattaacccgacagttccagcaacatagtagcagtacatgtagagctcgtcgaagttgttataccttgtcttcctaagatcactcctcatcccttcaatcatgtccctgaatggctgccacaacat

SEQ ID NO: 27 - RNA, 20 nt, Artificial Sequence
  Description of Artificial Sequence Synthetic oligonucleotide
aucuagaggguggacaaggu

SEQ ID NO: 28 - RNA, 20 nt, Artificial Sequence
  Description of Artificial Sequence Synthetic oligonucleotide
aaaccaaacaugcccuaggu

SEQ ID NO: 29 - DNA, 308 bp, Zea mays
tggcagatcgatctagagggtggacaaggttcctctccaacatcccggagtatgttcgtgagttggttcgcaattcccagagccaaggcagcactgtatacgctttcagttgttgctttagactcggttgcgatgcccatcacaggtacgctcattaacccgacagttccagcaacatagtagcagtacatgtagagctcgtcgaagttgttataccttgtcttcctaagatcactcctcatcccttcaatcatgtccctgaatggctgccacaacatacctagggcatgtttggtttcaattagttc

SEQ ID NO: 30 - DNA, 175 bp, Zea mays
ttgctggaactgtcgggttaatgagcgtacctgtgatgggcatcgcaaccgagtctaaagcaacaactgaaagcgtatacagtgctgccttggctctgggaattgcgaaccaactcacgaacatactccgggatgttggagaggaaccttgtccaccctctagatcgatctgcca

SEQ ID NO: 31 - DNA, 175 bp, Zea mays
tgcccatcacaggtacgctcattaacccgacagttccagcaacatagtagcagtacatgtagagctcgtcgaagttgttataccttgtcttcctaagatcactcctcatcccttcaatcatgtccctgaatggctgccacaacatacctagggcatgtttggtttcaattagttc

SEQ ID NO: 32 - RNA, 20 nt, Artificial Sequence
  Description of Artificial Sequence Synthetic oligonucleotide
cugccacucugcugaggugg

SEQ ID NO: 33 - RNA, 20 nt, Artificial Sequence
  Description of Artificial Sequence Synthetic oligonucleotide
uccauccauuccguccuacg

SEQ ID NO: 34 - DNA, 250 bp, Zea mays
atgtctagcagcgacccggaggagatcagggcgcgcgtcgtcgttctcggttcgccccatgccgacggcggcgacgagtgggcccggcccgagctcgaggccttccatctgccgtctcccgcccaccagcctcctggcttcctagccgggcaaccggaagcagcagagcaacccacgctccctgctcctgctggccgcagcagcagcagcagcaacacgcctactacatctgccggtggcggcgctgctc

SEQ ID NO: 35 - DNA, 250 bp, Zea mays
gagcagcgccgccaccggcagatgtagtaggcgtgttgctgctgctgctgctgcggccagcaggagcagggagcgtgggttgctctgctgcttccggttgcccggctaggaagccaggaggctggtgggcgggagacggcagatggaaggcctcgagctcgggccgggcccactcgtcgccgccgtcggcatggggcgaaccgagaacgacgacgcgcgccctgatctcctccgggtcgctgctagacat

SEQ ID NO: 36 - DNA, 303 bp, Zea mays
gctctgccactctgctgaggtgggagcagcgccgccaccggcagatgtagtaggcgtgttgctgctgctgctgctgcggccagcaggagcagggagcgtgggttgctctgctgcttccggttgcccggctaggaagccaggaggctggtgggcgggagacggcagatggaaggcctcgagctcgggccgggcccactcgtcgccgccgtcggcatggggcgaaccgagaacgacgacgcgcgccctgatctcctccgggtcgctgctagacatcgtaggacggaatggatggatggatgggtt

SEQ ID NO: 37 - DNA, 175 bp, Zea mays
ggccttccatctgccgtctcccgcccaccagcctcctggcttcctagccgggcaaccggaagcagcagagcaacccacgctccctgctcctgctggccgcagcagcagcagcagcaacacgcctactacatctgccggtggcggcgctgctcccacctcagcagagtggcagagc

SEQ ID NO: 38 - DNA, 175 bp, Zea mays
ctaggaagccaggaggctggtgggcgggagacggcagatggaaggcctcgagctcgggccgggcccactcgtcgccgccgtcggcatggggcgaaccgagaacgacgacgcgcgccctgatctcctccgggtcgctgctagacatcgtaggacggaatggatggatggatgggtt

SEQ ID NO: 39 - RNA, 20 nt, Artificial Sequence
  Description of Artificial Sequence Synthetic oligonucleotide
uacgucuggacgaggaguac

SEQ ID NO: 40 - RNA, 20 nt, Artificial Sequence
  Description of Artificial Sequence Synthetic oligonucleotide
ugcaccggccaucaccgcca

SEQ ID NO: 41 - RNA, 20 nt, Artificial Sequence
  Description of Artificial Sequence Synthetic oligonucleotide
caaacucguuugucugacca

SEQ ID NO: 42 - DNA, 385 bp, Zea mays
gcatgcccatgcatgcatcatccctggtcaaactcaaacactctccaccgtcagggaataagacttattattttattaacaattcaatttttatttattaattacgtctggacgaggagtactggtttatttgatgagagacatggcagtccaagtcaaactcgtttgtctgaccatggcggtgatggccggtgcaggttggggagcgcggcctgcagctctccggtgggcagaagcagcgcatcgccatcgcccgcgccatgctcaagaaccccgccatcctgctgctggacgaggccaccagcgcgctggactccgagtctgagaagctcgtgcaggaggcgctggaccgcttcatgatggggcgcaccacccttggtgatcg

SEQ ID NO: 43 - DNA, 84 bp, Zea mays
tggtcagacaaacgagtttgacttggactgccatgtctctcatcaaataaaccagtactcctcgtccagacgtaattaataaat

SEQ ID NO: 44 - DNA, 84 bp, Zea mays
tggtttatttgatgagagacatggcagtccaagtcaaactcgtttgtctgaccatggcggtgatggccggtgcaggttggggag

SEQ ID NO: 45 - DNA, 52 bp, Artificial Sequence
  Description of Artificial Sequence Synthetic oligonucleotide
  Description of Combined DNA/RNA Molecule Synthetic oligonucleotide
gagcguccaugaugguacucuucaauucacggcagacaaacgaggacggacg

SEQ ID NO: 46 - DNA, 54 bp, Artificial Sequence
  Description of Artificial Sequence Synthetic oligonucleotide
  Description of Combined DNA/RNA Molecule Synthetic oligonucleotide
gugaauugaagaguaccaucauggacgcucggcgggaggccgggcaggggggag

SEQ ID NO: 47 - RNA, 20 nt, Artificial Sequence
  Description of Artificial Sequence Synthetic oligonucleotide
aucagggagaaccugcugcu

SEQ ID NO: 48 - DNA, 15638 bp, Bos taurus
actcgtctctgctcactggggcatcagctggagtgactcaactgggattggaagacccggtcccagtgtggcatactcagtggccatcttctggctgctccgttggagataccagcgagaccctcggttctcctccctgttgccttgcacaaggctaggctgggcttctcgccacatggccactgggttctgaaaggaagcgggaaagtcctgacatgccaatgcttatccagccttggctagctgcctgcttactaatatcccgttggccaaagctagtcatgtggtcaaatccagggtcaatgtggagaccactcaagggtctgaatattgaagggcagcctggtccgttggagccagcagcgttaacctaccatggaagagtgaccggagagtcacacacaagtacatcctgccctgcctttgggggctcacagaggagaaaccttctggatactgggaatgttttactgcacatcccaatgcgtagatgtagtggataaatcgtttactcaggacagctacctgcttatgcttatagcatagacgtgacaacagaggctcagaaaaatgtagccacaaggtggtaagcccaatgctggggcccaggaaagggcattttgcatctgaaacaaatgtatttcttgctctcatttctgcagtcaagtcaagctagtgacaggtttcacctgtttcaggctcttttcttcccttgtttgtataaagcattgcagcagcttgcagtatgtcctcaggcaaagccctcttctctctgtgtttcattttccttctttctcaaacaaagaagctggattcagtgatctctacagttcctgccgattgttatattttactacataacatgatagtgagaaatggaaaatttcctgccatatcagtaaacaaggatgtcatagttatcagcgattgcagccctccaacctgagctgttcagccctgaaggaactcaggaaggaaagaatacctgccatttagcagccatcggactgtagccactccccacagtgagccctgaggaaactcaagatgtgaaaacataggattctggccccagatagctaaggtgtacatcaaaggaatgatttcagtggaaccagactcttgcatcttcctatacataaaaaggtgctaaattccttgaaaagtgttagacactcagtcctgtctgactctttgttaccccatggactgtagcccaccaggctcctctcccatggagttccccaggcaagaatactggagtgggttgccattaccttctccaggggttcttcccaacccaggggtctcctgcattgcaagtggattctttaccatctgagccaccagggaagtcctgagaaaccaggaagtctggtttctttaattaatgatggtttctttaattagtgattgaattgatactcctgactacctgcacttggttggaaaactcccatatatcctagttcctcctcttgcctcctctgaatagtttctcagagctatctaaaatgctatctcccgggctgcatttctcattttacccccaagcaaacttacctcccaactctaaaaaaaaattgagtaattaaaaaaaaattaatcgtatcatgttacattaacaatgaaaaagatgtgttcatttttttgacaagcatattccttacactagttgaagtcagtaaagctaattgataactaaatagcgaatctgaattttcttttgatgtgtctatttgttccctaacagagtctgaccatgtaaagtggaacagaatgagttaggactggctcgtggctttgccaaagccaggttttcctctactgtgaaacttggtctctttcatcaaaaggttaatttctacattatttgccttccaaggttgctgaaaagataaagtgagataagagaagtgaaagtacttagctttaattagggactgagggttttttctgttcctctaatcacattctactagatttctgacagccatgcatcagacttcagtttgaaaaaaaaaaaaatagaggaattcactctcagtcagatattcatcaacctctagagtattagctctgctcatatgaaagggtgaagtcttaattctttcaacattctctgtcattgtgtctgtgacccttttaataaaaccgaagggactgtacatggaaaaggaaaaagaagacatttttaagaagcaatagaaaacacttctgagactgtgatgtttttcttttgaatgtaccaagtagaaaagctttttttttttttttttttttttaactttagtttcactttcaaaactgcagcacattccccacatgaaaacgaactggatttggactaatgaaaaaaggaacggtcagctgtcgtcacagtaagtgaaggtacattttttacattcttactgggataggagctctcatgttgggtaaagctagaattacattggtttgaacattctttggtctttatttgatctggtgcttgaagtggagtcagaatcattcaacaaggagaacagaggaaaggggagagggtgaaggagtggggagaagtgtatggtaacacagagagcaagtgacagcagcaatttctaattaacgtgatgaaacattttgtgtggtattctttttctttgtgatgttctgtagtgggaaagcacactgaggaagtgatagaaatcattttgcacccgccttcttaaaacaaacaaaaaatcatatcttttcttaaagatacaaagccttctgagctttaaagaacttacctccttttctcagttttgttggctggagaggagataggggctgacattcgatagtgaatctgactatgagaaaacattcaggctttcctaagagtgagtaaacagctttcttcggcctaaaaaggaaaatggaagaagatgagacatggaacaatgtccagacgtgtagaaaggtcagaaatgagtaaatgtgggttgatcagcctcaggcagtactggtttggggaagttggctccaaattttcaagtaggattacatgttaatgtgtgtctgcgtaaatttaggggaaaatgttgtgaatttacaaaccaaactgtacaagccaaactataaataaggattaagcagcctggctccagacgccatgtcccaaacattattgtctttcacttggaaaaagaaatgtataatcctaggttttccaaaattgcaaggctcagtgaatctacttttcgtctctttcagcctttctttgtttctattacatctcatgaaacatcagctcttcattctcatctcattctctcgctgtgtttttgtttgttttaccgaagtacattctagaattaaagtcctttatgtaggttgcttagttataaacttatggagtctgtgcatatctcaaaatgctttttccccttatctttaattgttcagcctgacatgaaatttgaggttcaaaattattccccatgaatgttttgaaagcttatcactattgttttctagtgacttatgttgctggtgaaaagctttgtagccatcaggatctctttcctgtggaagtaatggatcatatatgttctccttagagagttttaggacgtttttggtgagtgatcacaattctgaagtttcataagagtatgtgtatttttcctttttcactcaccctgcctggcatttggagtcttttctagcagatgaagtgttatctccagatctgggaatgtctttcagtccttacatgttttataaatggcttaatccttctcagtaattctgatctctagctgttgcccttctggaagcagtaaatgtcacctagacaaggaggggagtgtatttcggggactgtgcctgcagctgctgctgctgctgctaagtcgcttcagtcgtgtccgactctgagtgaccccatagacggcagcccaccaggctcccccatccccgggattctccaggcaagaacactggagtgggtcgccatttccttctccaatgcaggaaagtgaaaagtgaaagtgaagtcgctgagtcttgtctgaatcttagctaccccgtggactgcagcctaccagcctcctccatccatgggattttccaggcaagagtgctggagtggggtgccattgccttctccagggactgtgcctatgaactggtaaatagaggtcctacccttatggtactaggagaagagttttaggaataggttttaaaatgagaaatgtaacaatagattaaatgaaataaaaaataactaatgagaaatgtgaagaggatgaggaaagaatataattaagttccttattttttggggtgtgggaagaaactacttaatgtctggaaataaagaaatggaaacataaggatatcattcaggtttgtgtaagtgaccatgagaattaaaagtagaagctgttaagaggttcaccttggaagtttgcaactgtgccgtgtgcttcacttagtcgctcagctgtgtccgactccttgtgaccccatggactgtatcctgccaggctcctctgtccatggggattctccaggcaagaatactggagtgggttgccatgccctcctccaggagatcttcctgaccgaggtatcaaacccaggtctctcatattggaggcaggttcttcaccactgagtcaccagggaaggggagagtttgtgggttccatttgtgagttttttgtgctgattcagtccacttgatgtattatttgtataaaacatacttgtttatcataaatgttatgagtatcaagaggacacatagtcacgtttggttgcatattattacaaggaataaaccaatgaatatgaaagcgtatgttattaatgctgtccattatgtctatgcttttctttataaggcaaaagcatgagcagacctttgccttatcatatccacttcttttcgggactgttgacctgttggatactgtgtacatcttctgcccacaaatgtactgttagacatgaagtagctgattgcagtcatctgaagttgacgcaaatacctgatgacctcccaacaaacataactgtgttgaatctcacccataatcaactcagaagattgccacctgccaattttacgagatacagccaacttactatcttggatgggggatttaactccatctcaaagctggagccagaactgtgtcaaagtctcccttggttggaaattttgaacctccaacacaatgagatatctcagctttctgataaaacctttatcttctgcatgaatttgactgaactccatctaatgtccaactcaatccaaaaaattaaaaacgatccctttaaaaacctgaaggtaagaaggaaatgaacattttcatgtaagaaatgaaaaatatattatcaaatattgttttcaagtcgtttatgcatcattagactttgtagctatcgaggtgaagaatgagaagtctgatctacataaatgggaagaggggtaccccacactcaaatatggccattatgattggaaatattttcaaaagagcaaatactgtcttaagtcagtttcaatcttaattataaagaactctcctctcttgcatccttccacttgaaacagaacttagatacaagatgtggagatgaatcagaatatcccatgatgaaaggcaaataaactctcagtactgctgatttagcatctgcagggatgaaaaagtgtcgagtccaaaatctgatttaaataaagttaacaaattgacccattataaagcaaattgttcaactaaaaattatctccttttgatttatgaaatcttctacagtattgcttatccaatggctaccacctttttcttgtcagcactttgcttgaattgtaaatccatgcaaaatgtatgctaaatctgaggcaattattaagtgcttttttatatcaaggttattttcagaactctttgttttcttcattttgtgggctaacaaaaaaactgagcatcccttggccttagataccaagaataatgaacacatttaatacgcttagacatatttataaaatgattcattggatagctggagacttgaaatgctttcttgtcatgcctgatgttgatgtggaagaaatgtctttggactcaaagacctcaaaatctggttaattgcttgcaagatgttatttttatttttaaaacataaattgaaggcttatgctattaatagacattacataagactttgccctaacatgtatcagcttttccttatgttttacctgagtggaagtttacatattcaaaagtagcacgaaatggtaaaacatcccaaagcacagacgaaacaaataaaaattagtattctgtactagtcaggatgcaaaatttacacaaccaccaatatttctcaatataaaaatccttttaaaatttcgtggccagtaacattctcttcatgcctcttaagacgctgtttcttcaactttttttctgttgtttagattcaagactaatgcatgactatttacttgataagcgacatgaatatttcctacatcataaatgaaagcttattataaataagtaaaatgcttattaagaatattctttacatcatgacaaataaaacttgcccttatattttggaagagaagtaaattctttgaattaagaaattcatctttgtagcaatggtgttttcagtaacaatcaggatttcttactactcctacagttttctagagaataattgagatgtgctattttgctgtccagttatttctaagatgcatgtacatttagaagaaccatttttgatgccaagactataacttacatacatattttttttcctcctcagaatttaatcaaattagacctctctcataatggcttatcatctactaaattaggaactcagctccaactggagaacctccaagagcttctattatcaaataataaaatttcttcactaacaccagaagaatttgatttccttggcaattcttctttaaaaagattagagttgtcatcaaatcaaatcaaagaggtaaggtaaaaattcctttgtacttttcagtgtgggcacaccctaattgtttcatatggatgagaatctaaagctagagatgaggaagggatgataacctttgtcatgttttccagaaatccttcacacatactgctttgggccattgccatcagcctcacaagaaagtgagtttggtagctattagaatccagaatctggggctgtgctgtcaaaaagaagtggagggtccactacagaccagaactttggccttggggaacttacatacttttcttggtctaatttccttgcctataaagtggaaataatgctacctcttgtacagaagtcctggaaaagttaataatagtatgatggttaagagcacagactttagagttgggttagctgactccctgtctgggctttatctgttaacaattatgtaattgggcaaatgattcaagttccgtctacttcagttccctaaatctaaaatggatatagtaattacacttccccatagggctgtgtggggaagaatttagcagtttctggcatgtaggaagttctcgataaatgataccttgcattatatttttacaaacatcttcctataacggagtaaacctaaccttttttggggggtctatgaccatcaatttgctaatgatttttgcatacaggtatgtctgtattttattcctttacaaaccaagaagaagagtatcgggaattctaagcaaaatcaaatttttttaaagctatgttgatttttcagataaaaaaagagactgtgaatgaaattatcctgagataataattaaatatcttactcaagctatttgtgttcgttttgaacagcatttttttgtttagaacatttattgtagaaatatgtaaataaataaatgtaaaagatgttaagtaatgagggaaaaaaatcatccaaatccccacgatccaaggcaaccagtgttagaatttggatatgtttgtatccagcttttctccatgtaataaaagaaataacaacagagcaactgaaattcatacagtacttatgtgtcaggcattgtgacaagttctttttttattaaccttattttgtattgggatatagccgattaacaatgttgtgatagtttcaggtgattagcaaaggaatacagcctaattttatacagttctgtaactaacttatgcttatcgtggttttacacaggagtaaattgagggacttcccctggcgatctagtggctaagacgcaacactctcagtgcaaggggcccagattcaatctttggtcaaggaactagattccacgtgatgcaactaagagtttgcatgccacaactaagtcctggcacagccaaataaataaataacttaaaaaaaaaaaaaaaaagagtaaattgagatgaggaaaagttaaggaacttacccagaatctcaggtgatgggctggtaacagagctgagtttctgcaagtgttagactagggcatgtactcttcccagctgtgctgtattgcttctctgaggatgatgatgcatcctgccttttaaaattaatatcatgtcatgagtatcctccatgtcattaagagttattcaaaaatttcattttagtgacagaaaaataaccctatcatatagatttaccttaattaatctgacccttaattaattaaagaccctgatgctgggaaagattgagggcaggaggagaggagagtgacagaggatgagatggttggatggcatcactgactcaatggacatgggtttgggtggactccagcagttggtgatggacagggaggcctggtgtgctgcggttcattgggtcgcaacgagtcggacacgactgagcgactaaactgaatctaatcattttcctgttttgtattttgactcacttatgataaagcacactttctttcttccatgtctttgttatttcttccattagccttaaggtcacagaagtgacatgctagaaaccagagcctctttgcactcccggtagacttgatctatcctctctggattccttctctcctgccttctgccctcttgttccagcactgagtcccagggcagccaaggcttccagtgagggactggggggcagagcagaagacagatggaaacagaataatgaaatagtcaggagaaaacttgttggaaaaacagagagtgtcaatggagctcagagtcacagaagggaaagtgcctgtcagtgaacacttcggtcaatgggtggtaataaagtccgctcattcattccggggtgttcactgagagcagtgatcaccaagacagaaacagcatctgccccagagcttactttgcaaagagtttactaataacagttatactcagtgtggtgaaataaaagggctaagtgtgtgtgttagcctctcagtcgtgtccaactatttgtaaccccttggactgtagcccaccaggctcctctgtccatgggattctccaggaatactggagtgggttgccattctcttctccaggggatcttgcccacccagggatcaaacccaggtctctcgcattgcaggcagattctttactgtctgaaccaccagggatgccacttaacctagtctgggagatcagggaagaccctctgaagaagtggcagggaggccaagatgtgggtgctgagtagtggccagtgaccatggaagtacaggctggagcaggaggcgggtgacccaggcacagaggaaggagctgggagagagagggatggagtgaaaacccggaggaggtggtgagagaggaaaggaaattgagtcaagaactgtgcctgttttccaaaggtcatacagactgctgtgttttcaatactaacctcttcttccatcccactcttagttctctcctgggtgttttcatacccttggagaattatctggcctctctctgaacaatgccaagctgagccccagtctcacagagaagctctgcttggaactgtcaaacacaagcattgagaatctgtccctgagcagcaaccagctggacacaatcagccacacgaccttcgatggactgaagcagacgaatctcaccacgctggacctttcccgtaactccttacgtgtgatgggtaatgactcctttgcctggcttccacatctcgaatacctctctctggagtataataacatagagcatttgtcttctcgctctttttatgggctttccaacttgagacgcctggacttgagacggtcttttactagacaaagcatttcactgacttcgctccccaagattgacgatttttcctttcagtggctaaaatgtttggagtacctcaacatggacgataacaactttccaggcataaaaagaaatactttcacgggattggtgaggctgaaatttttaagtctctccaactccttctcaagtttgcggactttaacaaatgagacatttctatcgctcgctggttgtcctctgctcctactcaacctaaccaaaaataaaatctcaaaaattcagagtggtgctttttcttggttggggcacctggaggtccttgacctcggccttaatgagattgggcaagaactcacaggccaggaatggagaggcctagacaatattgtcgaaatctacctttcctacaacaaatacctagagctgaccaccaactctttcacctcagttccaagccttcaaagactgatgctccgaagggtggccctgaaaaatgtggactgctccccttcgccttttcgccctcttccgaacctggtcattctggatctaagcaacaacaacatagccaacataaatgacgagttgctgaagggtcttgagaaactagaaattctggacttgcagcataacaacttagctcggctctggaagcatgccaaccctggcggccctgttcagtttctgaagggcctttttcacctccacatccttaacttagggtctaatggctttgatgagatcccagtggaagccttcaaggacttacgtgaattgaagagtattgatttaggaatgaataatttaaatatccttccacaatctgtctttgataatcaagtgtcactgaagtcattaagccttcagaagaacctcataacatctgttcaaaagactgtttttgggccagcgttcaggaacctgagttatttagatatgcgttttaatccatttgattgtacctgtgaaagcattgcctggtttgttaattggattaatagtacccacaccaacatctctgaactgtcgaaccattacctctgcaacactccgccccaataccatggttacccagtaatgcttttcgatgtatcgccctgcaaagacagcgccccatttgaactccttttcatgattaacatcaatatccttttgatttttatctttattgtactgctcatccattttgaaggctggaggatatctttttattggaatgtttcagtgcatcgagttctcggtttcaaagaaatagacagagcagagcagtttgaatatgcagcatatataattcatgcctataaagatagggattgggtctggaagcacttctccccaatggaggaagaagatcatactctcagattttgtctggaagaaagggactttgaggcaggtgtccttgaacttgaagcaattgtgaacagcatcagaaggagcagaaaaattattttcgttgtaacacagaatctattgaaagatccactatgcaaaaggtaggtgaacactatgacatttgaaatgtattcttatctttaaactgagcttttattttgttttaattttattttattgatttggctgtgtccagttttagttgcggcagtcgggactgttcatgcattaaaggggtgcatgggctctctaggtggcccatgggctcagcaattggtggcatgtgggcctagtttctctgcagcaagtgggatcctagctcccctgtgcatgcttgctcagtcacttcaggcatgtccaactctttagttcccccaccagggatcaaacccatgtccactgcactggcaggtggagtctttaccactgagccgccagggaagcctctgaactaccagaaggaaagtgaaagtcgctcagtcctatccgaccatttgcgaccccatggactctacagtccatggaattctccaggcaagaattctggagtgggtagccattcccttctccaggggattttcccaacccagggatcaaactgcccgtcccatattacaggcggctgagtccttaaggaagtccctgaaccaccaggaaagcccctgaattgagcttttaaatattactaacattattactccggagaaggcaatggcaccccactccagtactcttgcctggaaaatcccatggacggaggaggctggtaggctgcagtccacggggtagctaagagtcggacatgactgagcgacttcactttcacttcccactttcatacattgaagaaggaaatggcaacccactccagtgttcttgcctggagaatcccagggacgggggagcctggtgggctaccgtctatggggttgcacagagttggacacgactgaagcaacttagcagcagcagcaacgttattactcacaactcaaagtattttaaaaataaaattaagaagaaacataacctgggttttagtgacaagactaaagttaattacaaggagaatgttagattttagagtgttgggctgttaaaaactgtgtattgatgtatgacttaataatgtttttaacttagattcaaggttcaccatgcagttcagcaagctattgaacaaaatctggattccattatattgatctttcttgaggagattccggattataaactgaaccatgcactctgtttgcgaagagggatgtttaaatctcattgcatcttgaattggccggttcagaaagaacgggtaaatgcttttcatcataaattgaaagtagcacttgggtccagaaattcagcacattaaatttatttaaagactgaattaaaggagtaagtttcctaatttaaaaaagttccatggtaaatttacattttccttgacaataacataaatctgttcattcatattcataagtaagaatatcaccactatgtctcttctatggaaatatgtctccctatttcaggccttttgttaccaattgacataatctgacccaaaataaacatataagaatatcttttactaatgaacacacaggttactgaggcctatgaaaacagttttgaaatagtcctcaatttaaagaaaagttagaggtatatgagggtttgtgataaatatatctgttattttttttctggatgcactcagaataaaatatgacttttaagaaaagttagaggtatataagggtttgtgataaatatatttattttttttctggatgcactcagaataaaatatgacttttaatgggtacaatgctatttcatttgtgaaagggtaaaacgtacacctatatttttaaaagtttaattacagcaattttttaattgaaaactttgtgttttagtggtgcctgttgcagtgttgattttattaaatattaaattacattttagtatacacatgtgtgtgtgctcagttgctgagttgggtgtctgactctttttgacctcgtggctgggatcttcagtccatgggatttcccaggcaagaatactggagtgagttgccatttccttctccaggggatcttcctgactcagggagcaaacccgtgtctcttgtacctcctgacttggcaggcggattctttactactctgccacctgggaatcccatatactcaggacaaaaaacatttattcttcattttgattactttatacaatgaatcttttaatgaatacttatgtttcctgatatttggatttctgaggcattagaagaaacaattcttttgcttacaagtgtttcaggaaggcttacttctcaaaggttaattagtagattggctttgtggaaacaaaatattgtcttaccctggtcacagactcaggagatggccctggctaattcacttggttccactgggattttctgcctgccaatcttgaaaattatagtgattaacaaaatgttaactattcctacctgactcatttgaaaagaccctgatgctgggaaagattgaaggcaggaggagaaggggatgacagaggatgagatggttggatggcatcaccgactcaatagacacgaatttgagcaagctctgggagttggtgatggacagggaggcctggcgcactgcaatccatggggttgcaaagagttggacacagctgatctactgagctgaactgaactatttatatatcattagtgatgatgattgtggtataatatattcatgtagactatattattataaccatttaaaaagtatcctgtgtacaagttactcttatgattgttttgttttactttcattttacaatttaaaaagggtctaattattggaaattgacgtgaatagcttttttaaacctttatagttctggtattcaaatataagggagagttgaaatgatcatgctgagatactttctgtatctgaagcactcatttgtctgctgaatttaggaagcatgtgagatgtcacattttggagatttgatttaaatattctatatttcatattaatatgtatatatacacacacagaactattaaataggacagggtaggttcatgtaatcctaaataaagtacactcgctgtactgctggttctgaagaactctacagcaacatgagtatatggagaattcattttcactctcacatttatatgccacccttatgcttgagatgttagttttattgttttttgtaaagatttgttttctaatggtgaaaagtgaaagtgaaagtcactcagtcatgtccgactcttgcgactccatggactgtatagtccatggaattttccaggccagaacactggagtggatagcctttcccttctccaggggatcttccaacccaggggttgaacccaggtctcccaaatagcaggcagattcttcaccagctgagccacaagggaagcccatctgtgaaagaaccctgttttattttggcatcataaaataaaaatcacaactgcttttttcttgatgcactttgtatgttatcaatactcccaacctcattgaaacattttgaggtctggctgctggagctggacaaagttatattcacaaacttttacaaaatgtaacaacttcaaaacacttttaaatacttcaatttagtgatttttcaggctaccaaaattttttctcttttaaatgactctaaagtaagttcccggagaaggcagtggcaccccactccagtactcttgcctggaaaatcccatggatggaggagcctggtgggctgcagtccatggggtcgcaaagagtcagacacgactgagcgacttcactttcactttcactattatgaatttatcaatatattatcagtttgtgaaaagcaaagcaggaagtaatattgcgttttctctcttccttgtttatgaagtcattgatcacaggggcacaatttaaattgag

SEQ ID NO: 49 - DNA, 62066 bp, Bos taurus
  modified_base (1674)..(3100): a, c, g or t
  modified_base (3165)..(3165): a, c, g or t
  modified_base (3208)..(3208): a, c, g or t
  modified_base (4282)..(4381): a, c, g or t
  modified_base (10305)..(10305): a, c, g or t
  modified_base (12031)..(12212): a, c, g or t
ttagcaaccaaacaacaaaatatgtgagaaagctattaatttttatgtattatttgcatggcaaagtggtatgataagaaggcatttaatttttgcttgtgtgaatatcatttacatggtatgtgacctgaggagagtccagtatcttttctgcattaattccattatttgtaaaaaaaaaaaaaaaatttaaggctaagaagatatttcttcgttctttttagtagtcaatgaaataatacatattatgcagttagaacagtccagttcaaaataaactcccagtaaagatcatttattattgtatctagtagtttttcagctgacctagggcactgcttgcttttcaatccagtagatatcactgatggttagcaactggttttcctgtggcactcttggcaattttcaaattctctctggatcgactaggtggacttttaaacataaccaccgatattttgactctaccccctaaaaggcccagtaatcacttcagaagggaatgagtctcccagaaaccccactttgagaaacggttttacctgaccctggactgcgagtatagctgccctcatttcacccaagtgagagaaccaagtctgcccctgcttagaaaccagagtccctttcagctgagcctaagagagaacaagggaaatgtagacacgtctcaaagatacctgtagaatgacggttctcagtcaggggcaggggctgtgttacaatgtctagagacatctgtgattgtcgaggggcagtgtggctggcatctcgtgggctgaagtcagggttgctgctcagcctcttacaatgccacaaagatgtagctcaccagaaatggcaggagtgcgccagttgagaaacttctagaaaaaaacatcagccagccaagttgcttgaatttagacatgaggtttccctgactgtggtggtagaaataaaaggaacctgctgcttctgcaaacctcatgtctgagatggcataaatagtcacactcctccccctaccccctaaccagtctttgcacttagaaagttcacgactggcagcctggagtctccatgacttaggtaggttttccacagctcatctcttcacctcagaagactccagacctggactcactccatgcagcaaagaaaggtattttaaacattgggaaaaactcagatcatttaagtacctagatctgtgagccattctcagagagaaggggagagaggggaagggacccaaatcatatgcaaaaaatttttttttagaatctgaagaagagttttggtagtctcaaatgtcacttctttgaagataaagtcagacttttacaataatgctggaaatggttttatggagtgtgtctctcgtctgtatatttgggcttgatacttggacttgaggaagtaaatactattaatacgtgttttcaacagcgtatattaaagcagcctttccagcacaatttgaagcatcaatcctttgctgtctttgcaaggtaaagtttggtgttttctctttatcttagttgatgctactgggtccatctcaggctgatcttgacacctctcatgttctgctctcttcaaccagacctctctgtttcattttggaagaagactgaaaatggtaaggacagctaagacatccctaaaaagtgttatttgtcctttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttgtgggtcgcatcaagggtgccaagtgccctttcgacctccaattcctaacgtgggacttctcntgaggcgctgtagcgggaaagggcttcatcttgcgatgacggnggagccacgtggtttttctcgagttacggcgggattctcgagttacgacggggaattcaggctgcctcttgtgttggcccaggcaagtccaatcttccattcgagttgcgaaggaaagctgggcattgctctcgagtgcctgcagggccaatagacctcatctaggcttgtgtccagaagccaatgttcctctccagggacgacagggatctcggggttgcattccagacgcacccggggagacaggcattcatctcgagtggaagcaaagaaccccgctctgctctcgaatcgcgacgggtatctcttggagctcactgggtggactcatgggagtcaagcctcctgagtcgtttggagagaggccgcgagcttggtctctaggccatgcaggagacgaaggccgtcatctctcgatgacgggggaatctcggggttgttctcgagcggcggccccagtgtgcggtttctcacgaggtacgacggcgaggtcagtgagcctctcgtggggcgccagggaagtcgggtctccatgcgagtggcgagggtgagcgcgtcattgctcccgagccatggtaggggactctggcctcgagacgtgttgaagaaggtctctcgaggtctttcccgggttgaggcaggaaaccctgggttcactagacttgtgcaggtgacctcagagggcttctcatggtggctcagagaagccagggaaactggaggtgagagggcccctcgggactccactgggtttggtgcattggaagagggcctcatctccagttgaggcaggaaccgcagggtacctctgatttcagactccgatcgcagggtccctgcagactggggacaggagagtcaggcctcgtcttgggttgaggcatggaactccgcttgcctctcgagatgtccccggggagagaggccgcttgtcgagctgtctttggaacctggggttttttccgaacgatgcacggaaaactgccccttcgtgttgacttcattcacagggtggagttcggagaggtgtccgggcatcgggtctatcagaggggaccgggaatcgggtcctannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttgaccccatggactgtagtctaccaggatcctctgtccatgggattttccaggcaagaatactggagtgggttgccatttccttgtccaggagatctttctgacccagggattgaacccgggtctcccgcattgtaggcagacactttaccatctgagccaccagcaaagtctgaaatcggaaaagagccacagaagaaaaggggatggtatccaagggaggccctggggagcagacctacagaaggactcctatgcaatctgttttctcagaagttcttccccctagaacctgaaacctgagtgggagcacagaactcatttgtctctcctgggaacaagcacagccaggatgggactgtccctgacttgtattcagtcgctgagtcccagcccaggcaagctggatgccggacaagccttctcgtggctctctgcgccagctgcctggaactcacagatgggttctgatcaagcaggagaggaagaaaactgagtcaatgaacaaagccttttaaatgtgttaatgagaaataatacctttcaggcttccaaacagaaaaggtatgtttgagaagatgattgtaaaatgtgaaatagtcctttattcccaaatttcccaaagtatggaaacttcccctttcaaaccatggggaactacatggcttcagaattaagctaggaccttcgcttcaactaaagaccatttttaaatgtaagtgttcattcagcatgatatgaggtctgtttttttcatttcatacaaataacccacattatttttttttgcaataattgccaaagacatacatctttcaggaatttgatttgtaaaataacataattcaccataataggcctggctttagtcatgaatcatagctgattacacatgcaaatatactgggtaaatttggatgcccaattagcaagctaattatggtaaaaatgggactgtgattttgtactcacaggtgttctgttctctcagtaaatcacctttttgttaaaatggaaaatgtagaattcacactgactagtcatcagacagtcatttgtgttttttttttaataatggtcaattaccaggtatttattatgtttagatgccaaagaagatactttgtttatattacatcactgagccctgacaatgaagtttaaaagacataaatattcccattttgctgatgagaaaacagggatcagaaaggcagtttcaaaccaagattcaaactccattttaaccaatcatcttccagttatttcatattgttccaacaaatgcacttggtatgctatatacatgcattttggttaatttccttttcagttaaaggatttttggtcatctaatgttttgatattgtggtatttggagttttcataataaaaatgtccaaatattatgaaaaggtatttatgatttgagtgggaccaactcgagagaaaaaaagagacttttcagatcatatagttcttaggtgtgcattcatatccagtaatgctatgctagcatagttcatggatttataaatttaaaggcagactgagctttagcttcctaataaagaaatatatgccaaggcccccagatgcaaacatgctggaatcaccacaaaaatggcaacagaggaggaatagagagtaaaaggagatgaattcaaagagggaggagctgtagtcccacattgaggattcctacagggccttgtcagccatcccaacaaggaatggaagatcgattcattgcaaatttttaagtcaaagagtgccatggtctgacttccatgttacaaagatcatgctggttactgccttgaaagagacaatgaggggtgaggttggaagcgtggagaccagtcaagaggctatgatggtaattcaagcaaaagccccagctggctggggtagaggtggtgagaactagccagatacttggatagatttggaaggtgaagccaaatcaaataggactttcaagttcttggaagaaaatgagaagaggggttggaggcagccagcatttctgagaagcttcctgcaaaggggaacaaagatgctggcggtagttcccagggccaagagggtcaagaggatttatttgaggatggaaggaataacagtacattgtaatgatggtgagagggaaaaaaacttttttccaaaaaccccatcattaataatagtaccaatgacaatgcaggagagatgggagaatgctggtgtgtggagcccagggaaggaagcaggcaggtgacccagtgcactagtggagagatttggctttagcaaggagcctaacaattccatccttagtataagagtctgcaaactatgcaattatctcccacctccttagaagctgggcaggcttttgggactgtctggaggacagcagcaaagtaggactacacaacttcccaggcagggacagacaaggtgggagactttcacggcacccagtgctccatctttccctgtctctctctccacctgaactggagtcctgagccaccagccttggaagaagcccggcgacagcacatctgacatgttggggagaccacccagagacaggggcagcctgcagctgcggagcttgcccatctgggcaccagacctgtgtgggcggagcccagcccctgacattgactgcaaggcgagggggaacacacaccagcgccagagtcaccttgctgagcccagtcaacctgtgggcccgccactcctaacactcgaggacagtggttgtttcgagccagtgggtttgggggtgatctgctccgccgtggtgggtaaccagaccataagtcaagcaggacgggcaggtgtagaagctctaggtgggcacataagtggggaggagcaggtgggtaatctcttccaattgctgtgatgctcttggggagacggtgagcagccggcaggaggggctggtggggcgagggagaggggaaggtgaagagagtcctctaggcagaggaagtcaggtgtgattacctggtggcagggaaaaacccttcaccattcggtatttcatctgcattcccctcgcagtctttattatgtaattttccatagcccctgatctggttggatgaaaggcctacagaaaaattcttcacagaaggaaattgacctcaaggtgagaacgagtgaggccaggggacactcacggttacactcagttaagagaagatctgctccctccttcgctcatctacccaacagactgtctgaaaggccagcatactgggcatcctctgtgggctgtgacatgccagccttcaagggaccacaggtcagtgccctcagatactcctcccatttattttcaagatgagagagcaggaaaaaaaaaaaaaaaaaaggccacattgcttgagttgaaaaggaagtgttggaatatgcatttccagtggagggagtcactgaagccaagctactaaagccaagcagaccacggcgcagtggggatcccccacgtggacgggctgggttgaagcagaggtgatggcaggctggagaatcatgagacagggaccaggaagagacaaggagtggggcgccattgtgggaaatccttgaattcgaattcagtaaacttttgtcatgttgccgtgacactgaattgtttgataactaaataagcgttgtatatatcaccctaggggctaaccaaggtctgcaaataatggaacttaacagatgtgatattgttttataggccagactccactgggaatttccctactagacttacaatgaccaaatatcccctttttgcaaatttttgaaatatttctaattataggctaacctctgaaaagtgtaattaccatagacatcaaagtttcattatataacaaccaaaagattaagtacaaggacataacccatttacttaaaaaatagactttttatcatttcctttctcccattttagcccaacagacctagttttcaaaataaaagagtcaaacgatataaaggagtcttatataaattagaaattcaacagctaccttgcatcataacaggcaccaaagaccagaagaaattacatcttacttgacctcataccctaccctcttttttccataaaggaaaactgtcaacttggcacactttttcagtaaagagccagtcagtaaatattttgttttgcaggccatacagtctgtatcacagctactcaaatttgccactgcagtgcaaaagcagttatagacaatatataaacaaatatgcaaatactaaagctaattgtgggcacaaaaatttgaatttaatataactcttaagggtcacaaagcagtatacttcctttgactttttcaatcatttaaagagattaaaaaaacctcttagcttgtgggctctacaaaaacaggtggcaggctggctatgacccacacccattatttgcattgcaacctaaactttgagggacttctgagatataaaaatgatctattcttggggtgcaaagacgctggttggttggttgatctgtaatgattgcttttaattttagtatgattttccctatttttaaataatcagttttgattagtttagttaatggactgctttgtttggccaggaaggagtgtgctatatctaagagcagaataatgtttttatttgtaaaatcgcttaactactgttatatcttggcatgttgcctctaacactcatctccctcttcctgtatctatttcaaatagtgagaagcacttcaatactgccagatgcttcatctagtaaagttttgattttaagttcttattctgtcatctaagctgtcttggacattagtgaatcactttccctcatgagcctctgatttctcaactataaaatggtttgaatacagggctcttttgtgaaactattgagaaaactattgagaaactattgagaaactattgagaaaaactattcatgcaaactgtcattccagatgaaccaccatttagcaagcatctagtatgtgcattaaaaacatcaggaataaacaatacttcactactagattgctttttccgtttaccattttatcatgagtactttcttatgtcattcagtattcttttaaaaaatatgagacttccctggcagtccagtggttaagactccaggctctcaattcagggtcacaggttcaattcctggttggggcactaagatcctgtatgtcaaccagcacagccaaaaaaccaaaattaaacaaataaataaacagatgatctgatggttatataacatgctacttaatgtcagtcagtcagtcagttcagtcgctcagttgtgtccgactctttgcgaccctatgaattacagcacaccaggcctccctgtccatcaccaactcctggagttcactcaaactcatgtccatttagtcggtgatgccatccaaacatctcatcctctgtcgtccccttttcctcctgccccaaatccttcccagcatcagagtcttttccaatgagtcaaatcctcgcatgaggtagccaaagtactggagtttcagctttagcatcattccttccaaagaacacccaggactgatctcctttagaatggactggttggatctccttgcagtccaagggactctcaagagtcttctccaacaccacagttcaaaagcatcaattcttcagtgctcagctttcttcacagtccaactctcaaatccatacatgactactggaaaaaccatagccttgactagacggacctttgttggcaaagtaatgtctctggaggaccacaattaatgaaccctttcccaacacttgacatgagatgttctctttgctatgctgcacagactaaccttcataaattgcttgtgacatccctgatcatttctttaagaagagtttctagaagtggagtttccatatcanagggtctgaactataactattaataagatcgaataatttccctatgctttttgaccttttctggttttttaaaaactgttttcatattttttcttataaaagatatcccaattttcttcttaaggactacattatcgcgcttgaagcccacattgaagccctgagtaggagtgtgtgctgggctcggtctaggtagaacagaggggtctccagcatggcttccagggttgagctgcccagccaaaaccagaagttcttagactcttgaagccccggctccctcatctatgacttacaaggctgcttagagggtgaaatcgaaacagagacgtccagccctaagcacgatgctcactatagtaagcagtcaataaatgtgagctctctttactaccatcaatcctatcacaccatccctcaaggggcacaggcagatgtgggcctcagtttgcctataaacagacatctgagtttctaggaaatgaaactctacactgttgcttctctgtctcttcacacagtgtcctgtggttaagtcttcattagaccttagacatgaaatgagcctgggagcccaccttccaatctagaagtcagagtgggttcctggtacaagtactccattgtgtctagttggggtgacaaaataatttcagcaagaagtcactggtgtctttttctgatgaattttgtggcatgcctaataactcataccacatgcaaaggctctaaatatacattctgttatttgagcctccacaatggcccagcaaggcaactagagagccatagtaatgttcattttatagtataaaaaaaaaagctgagtttcagggggcttaagtgataaacctaaagatccctcactagtaactgggatagtgaacatgacagagtctctctgtgactaaactttagtcaagctcctctgactcagccttgatatttgggcttttgtgttcatgtggccatttttcacaaggatcctgtcaggttggtttaggaagagcccctcaccacagtttcattatccttgctatctgatcaaataccccatctctacgaccccccagggaacagtccatcttcctggcctgctttcagcaagaaccctgtcaggtgggtcgagtcagaaccaccccacgccctgccacccctgatgtttcctcttagtaattttccacccgctgacctccactctgctcctggactataaatcccacatgcctctgcactatttggaactgatcccagtcctatactacagtctcatttccccgattgcaacagttcctgaacaatatctgtttttacagctttatctactgtctggttctggttttcttggactgcatgggagcccaggctccctgtatttctagtcctatgcatcctgcccgctccctcccctgcatacttgtttagacaccaccaaatgctagaaaaacacaaaggaacctcagtaatttgtatggtaataaagcccgaaacgagggcttccctagtggctcagttggtaaagaatccgcctgcaatgcaggagacctgggttcaatccctgggtcaggaagatcccctggaaaaggaaatggcaacccactctagtactcttgcctgaagaattccaaggatggaggagcccggcaggctacagtccatgggaccacaaagagttggatacgagtgtacaactaactttcaaaaccaaaaacgtggggtgaaaaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncattttcttctccaggggatcttcctgacccagggattgaacttaggtctcctgcattgcaggcagacgctttaacctctgagccactaggcaagccctagaggacagaggatcctggtagactgcagtccatgggatcacaaagagtcagacacaactaagcgacttcaccttcttccttccttccttccttccttccttccttccttccttccttccttccttgagaagccctcagccctcctggtccctcctcactgccttatcaaaaatctgatctttggaaagactttactgtttgtgatcattgaatgtgagcctccacgggattttaagaacgttcaaagccttggtttacttgcccgatatttgtcttgtgctcactaggtggcaggcatcatcagaaatggtcaagacagataaggccacagcccctagagtgttttacctcctagagggagacatgggtgaattgatacctagacttctgggaaggtaagccaggagaattgctgagggccagcactgcatgccagatctcctgacaggcaagttctatagtttctgcatctcagtgtcctcattgtcagtgaacctatttcttggggtgaggatgaaataattggctaaaagctattggttgaaattagcggcccatgaggcaacttagaggtttacaggtgatatgatttcttctcatatttggcagacttgaactggatttgctttgaggtaattccatgtgcaaatggaagtttttgcccagaccggggccctgaaaactacgacccatggaccaaatctgtcctgttacctgtttttgtgagctgagaattattttttaccaccttgattcataatgttctaagtggttatttaagtacccacattaatagcttcaattatgctgggaaagattgagggcagaaggagaaggggacgacagaggatgagatggttggatggcatcaccaattcaataaacttgagtttgggtaagctctgggagttggtgatggatagggaggcctggcgtgctgcagttcatggggtcgcaaagagtcagacacaactgagcaactgaactgaactgagtctaaaatattgactgtccggccctttagagaagaagtttgccaacctctagtctagaccagctctgtctactagaactttgcacagtgatggaaaatgttccgtatctgtgtgcttgaatacagtaaccacgaacctgaggtgactttgagcgcttgaaatatggttagtgtgactgagagactgaatttgaaagttgcacttcattttaattcatttaaatagaaacagctacacgtgaccagtggctatcacattggccagagcagctttagaccacttaagtctgaagattctgtattccctcagtgttcttgtctctcaatttaatcaacttcccagaggctgtctgagtcaagaagaaggcagcactcctaatcagattttgggggccctgggctggctttcactgtcttctagaaaatgcttaatggaaagtgctagaaaaatgtgtgtgaacatacacttacttcctcttaaagctgtatccatgacatctgctattgtttggggtaaggcagttggctttttcaaacccagaaggccccaaattaaggaagtcttaatcatctttagaattcaggctgcaggcagtgaaaacctcacttagcgaagctatgtttctttccatccatcttatagaactaaaactaaagttagctttccagggcacccaaagctggtgctctgggacaacccagagggataggatggggaggacggtgggaggagggttcaagttgaagggggacacatgtacacctgtggcagattcatgttgatgtatggcaaaaaccatcacaatattgtaaagtaattatcctccaatttaaaaaaaataaagttagcttcccctccccgctaggctaacttcagccctagggcatttctttcataaaattgcctggaacagttaggagttaatcatcacatgtcatcattaggaatattgtcaagcaatttccccttagagtaaaactgaagttggccttctgggaatgtcatgtaagtatggtgtattacgtatttcaaagttgctaagacagtagatcttaaaagttctcatcagaagaaaaaaaaacttggaactatgtgtggtaactaaacttactgtggtgatcattttgcaatatctcaaatcattatgttgtacacctaacctactccaaggaggatttcttttaactattttgctgtatttcactgttcactcatttatgcatttattctttcattcatgtattttgcaaacataatcagcattaagtgcgagacattgttttacatgagggcacacaaaaccctgctctctcgaagctaacatttgcatgcaaaatatatataaaacaaataagcacataaaccagaagcatataaatgatccacaggattatttctgattgtgctaaagactatgaaagaaataaagcacagtagtctaatacagagtacatatacctctctctgccacagttgacttatctgtaaaatggggataataatagaggtctacaattccagaatccctaaactgaaagctttgtcctaactcagctgatggtaagcacaacttaatctgaactcatttggagatgaatcttagaccagaaccagatgatctagtgtttgttgtttttgttcttgttttttaaatcccacttagagtggacaatcctgtgctttattgcacaaatttttatgtgtttgattatgagatgctgccccagattccagttggagtgttacataatatgaggtacatacatcacattgccttctaaaatctgaaaaattctgagttccaaaacacatctagttgcaaaggttgtggataagggaccaaagacctgtaatgctgccctcatgtggttaatatgaagatgaaatgagtgagttaatatatgtaaagcactgcaaactttgcccaacatatgctaaatgctatctaagttaattactgctcctgtctttatcactgttattacatgggagggtgtttagacaagttgctcagagacagcctctctggggatgtgtcatttaagcagacacccaggggatgagaatgacctggctgtgggaagaggaggagaaagatcatttcaggaagcgggaacagcaagtgccaagactgactcaaatgcagaacagaaaacaggccggatatctgccatgtggtcaaagaaggggaaaagtggttcaaggtcagtgtggtaggctagagtcaaaacatgcatgatataaacaatggcaaagactttggacttcattcaaaacataatgagtgatcactgtagttgatctgattcatgtttataaagactgctctgatgctcttgatggatgaattgtagaggaggatgggagtcaaggagaccaggtttgagtccagtgtgatgatagtccaggtgaaacctcctggcaaagtggagtagggggtgggagtagacaaggagaaaagtggacaggtgcaagaaaacaggtagaaatgggaggaaaagacttaataaaatgataataaattaacaagctttaggcgtgcaaagatgtgtgaagaagctctttgaactccttctttaaccatcttgcagtagagaaaatgctgagggaaaattttgcttatttatcccctattaagggaattaagggaagtaggcagtagatttattagatatgagcatgactggttgattcagtcaggtgaggggatgaaagcattaactgagctggaaagctgttgtagagacccaggctagatcatcccatggatgaacacaggcgaaatgaaaaccagtgttctggaggaaaatattgatagaaattctgaaacttgagagcgagtctatggtaaaccaccccctatgcaaggcaagctcgagttaatagggtggttagtaaaactcttcattatgtcccatattccctcttaaggtcttttttgcccagagtccagttgtttctgatactaaacatagatgcccaagaattacccgtaagagtaggttcccacatgtcccagttggtctgagagagtctttgtgcctgtagtcttggcataaattattaaagcccctcccctttcattcccatgagtatcctgtttgatcaattggggatcacccaggtggtaccctactatattatcccctttcctaatctgtgtaatgtaaacccaatgtgtagggaaaatgttcatccccaagtcatcttggagataatttccaaaaatagtccaagaggaaaatgggcttatagatagcagtgccaaaatatcaggagttgggtgaccataaacagatcagttaatatcgctgagctgtgtctcagtctgcaaagtgtttccctacttagcatattaagaagctcagatgtggtaaaggactcttgtaatgtgtaccaggtgggcttaacaagcaaatgttaattgcactaaaggttttcttaccagatgtagttgcatttcagactttggcagcatgttgacggctctttcattgtaacattataagtaacagggaataggaaaatgctgcttcccctatctgaaaagcgatataacaaaagccaagatgctgaaagcgaaatatgtaagtttttatgaacctcagtaattgacaactacagtcatggggtgggggcagggatgctatttagacagttgagaacgataaacctgagctgttgatttttcaaatggagcaagagagaggcagcaaacgggagttttaaattctggatcttggtgtattttaaatgggtgatttattcttatactttcaggtgtttccaatgtggacattgaagagacagtttcctatcctttttaacatgatcctaatttctggactccttggggctagatggtttcctaaaactctgccctgtgatgtcactctggatgccccaaatacccatgtgattgtggactgcacagacaagcatttgacagaaattcctggaggtattcctgccaatgccaccaaccttaccctcaccattaaccacatagcaggcatctctccagcctcctttcaccggctggaccatctggtggagatcgatttcagatgcaactgcgtacctgttcgactggggccaaaagacaacgtgtgcaccaaaaggctacagattaaacccaacagctttagcaaactcacgtatttaaaatctctttacctggatggaaaccagcttctagaaatacctcaggatcttcctcccagcttacagctgctgagcctggaggccaacaacatctttttgatcatgaaggagaatctaacagaactggccaacctagaaatactctacctgggccaaaactgttactatcgtaacccttgtaatgtttcatttactatcgaaaaagatgctttcctaaatatgagaaatttaaaattgctctccctaaaagataacaatatctcagctgtccccactgttttgccatctagtttgacagaactctatctttacaataacatcattacaaaaatccaagaagatgattttaataacctcagtcaactacaagttcttgatctgagtggaaattgccctcgttgttataatgttccatttccttgcacaccctgtgaaaataattctcccctacagattgatccaaatgcttttgatgcattgacagaattgcaagtcttacgtctacacagtaactctctacagcacgtgccccaaagatggtttaaaaacattaacaaacttaaagaactagatctttcccaaaacttcttggccaaagaaattggggatgccaaatttttacatcttcttcataaccttgtcaacttggatctgtctttcaattatgatcttcaggtctaccatgcagttataaatctatcagatgcattttcttcactgaaaaaattgaaagttttgcgaatcaaaggctatgtctttaaagagctgaatagtttaaacctcttcccattacataatcttcccaatcttgaagttcttgatcttggcactaactttataaaaattgctaacctcagcatttttaaccaatttaaaacattgaaattcatagatctttcagtgaataaaatatcaccttcgggagattcacctgaaggtggtttctgctctaacaggagaacttctgtagaaggccatgggccccaggtccttgaaacactgcattatttcagatatgatgagtatgcaaggagctgccggtccaagagcaaagagcctccttctttcttacctcttaatgaagattgttatatgtatggacagaccttggaccttagtagaaataatatattttttatcaagccttctgatttccagcatctttctttcctcaaatgcctaaacttatcaggaaatagcattagccagacgcttaatggaagtgaatttcagcctttagtggagttgaaatatttggacttctctaacaatcggcttgatttactctactcaactgcatttgaggagctgcacaacctggaagtcctagatataagcagtaacagccattattttcaatcagaaggaattactcacatgctaaattttaccaagaacctcaaggttctgaggaaactgatgatgaactataatgacattgctacctccaccagcaggaccatggagagtgaatctcttcaaatcctggagttcagaggcaaccatttggatattttatggagagatggtgataacagatacttaaaattctttaagaatctgctaaacttagaagagctagacatctctgaaaattctctgagtttcttacctttgggagtttttgatagtatgcctccaaatctaaagactctctccttagccaaaaatgggctcaagtctttcagttgggaaagactacagagtctgaagaatctagaaactttggacctcagcttcaaccagctgaagactgtccctgagagattatccaactgttcccgcagcctcaagaaactcatacttaagaataatcaaatcaggtgcctgacaaagtattttctccaaggtgctttccagttgcgacatctggacctcagctcaaataaaattcaggttatccaaaagacgagttttccagaaaacgtcctcaacaatctgaacattttgtttctgcatcacaatcgatttctgtgcaactgtgatgctgtgtggtttgtctggtgggttaaccataccgaggtgactattccttacttggccacagatgtgacttgcatgggaccaggagcacacaagggccagagtgtagtctctctggatctatatacctgtgagttagatctgactaacttcatcctgttctcactttccatatcagcagttctctctctgatgatgatcacaatagcaaaccatctctatttctgggatgtgtggtatagttatcatttctgtaaagccaaaataaaagggtatcgacgtctgatatcacccaattcttgctatgatgctttcattgtatatgacactaaagacccagcagtgacagagtgggttttggacgagctggtggccaaattggaagacccgagagagaagtgttttaatttatgtcttgaggaaagggactggttaccagggcagcctgttctggaaaatctttcccagagcatacagcttagcaaaaagacagtgtttgtgatgacagacaagtacgcaaagactgaaaattttaagatagcattttacttatcccatcagaggctcatggatgaaaaagtggatgtaatcatcttgatattccttgagaagccccttcagaagtccaagtttctccaactccggaagaggctctgtggcagttctgtccttgagtggccaacaaacccacaggctcacccgtacttctggcagtgtctgaaaaatgccctggccacagacaatcacgtgacctacagtcaggtgttcaaagagacagcctagcccttctttgccgaatgtgactgctaccaaggagaagcttggctgccttgataggctcacccatgtgttgccagaagagcgttttaagattcttcaagccctgggattgcccatattggagaggagtcaccaatacatgacaaaggaagtggaaaaatgggatttatataagcatcaagtcatctttctcagctctctgtgtctccatttgcacttgagtctttgtcttctgcccttgcataaaatactgttgggagaagggtggcaagtagaggatgtgggactttgattctcctgtaattgtgattatttcacatacacacagtcaccaagaactgcacttctacccttaagaggcactggtatgtatagaaatagggttaaaaaaaggctcagagtctgcttatatggcattaaaatgtaccagttaattagtggtaaaaataaagacacagttaactcttcagccaattgactcaaccatctagtgccccgttctgtgcagaccatgtgctggcccccagcaggtggttgcagctcaagtgctccttgctctttctctcctgggcctgttattgggctctttggggaaacagaaacatctttgtcatcgatggatatagtctacttacaagtgggagaagaataaagcactctgtatgcaaagtcatgctctttacttttgatgataatttacctgcttaaatatggctctggactaaatgtttctctccccccaggttcatacgttgaaatcctaatatcccaatgtgatggtgttgggaggtggggtttttaggaagtggttaggtcatgagactggagccctcgtgatgggattactgccttagaggaagcagtcaggagactaacttgctgtgttcccactgtgtgagggtatggagagaggctgccagtctataacacacaagaggaccctcagcagaaccagatcacgttggcattctgatttcctccagactggcttccatgcttcagacttgtgagaaataaatgtttgttctttaagccatgcagtttgtgatgttttgttacagcagccgatttaagacagattttttttatctgcatggcaaagtactgtttccaaggaaaagttgcctcatccgaggatctttcaaactgctttattaacttatactgcatacttgtaattatctgaaaacttgatccatcaatgtcagacagctttgatgataaactctaaaggagaaccccaagagcatatatttatttagagttagcaagcagggatgacagtcacatggattcctttaaatttggaggctgggggaggaacagaggtttaccaccattaacactgagaaagcttgagtcttgaactttggcttcagtactcaaaaaaaaaaaaaaaaaaaaaaaccaaaaaaaaaacaccagaaagaaccaagatgttctgaagacacatactttctaagtgtgtataaatttgcatgagtccaaagtctgaaatgtgacctgtttgtttttatttcatgaaaaaagttatctacagaggtgtctgtgccctttagaagatagcttcaaatcccagacttcaaattatctgggagataactgtgttttcttcatatatggaaaagacaaaggttttactctgaggatggctatatgtattatttccaaaggacatgtaatcaataagggtcacaaactcccaaattaatctctggaatccacagagccaacgatctgagcctcagtaagctagattgtacttcataggtgcttatgtgaaaacaaagataacgtcataaaaattggctgttggcatcataaatttcttctattaaagcacataactatacaaggtttaagttattttggtgatttataaaccatgaagcacttgaagacaaacatcttctgaaatgaatcaagaggaagggaaactgacaacaacacaactcagaaaccacagcatatttcaacacgaggttgtgcacagtggtttgttgtaggaaatgacccaaagcacagtaacagatggttttcattttaattcctttgtatcttgacaagtcactcttttaatctcctgagggtcctctgtggagtcctgtaccattagagccattcatagatggcttggatcgaaagccaacaactcaggccaccaggaaaacatgactgctaggtcccctgccaagctctgagtggggaagagactccggattgtccatgaatcccatgctaccatgaaggggttcccaacctcttccagccctgaacctgtccttcttcctcagatagagactgtaccttagcacgacttggacattcgtaggatccaagccaagagaggcattgggcaggtaagcgttatggctgtgaaaaaggcgaaacacagagctgcagttcatagagcagagaaagtctgagtctgactgcaaccccacggctaagtcagggtctcttaaggtctcttggcccatcctcacgtctgctcccagctctgtccaaacagtcttaggtccagctgatctcttacatgtagtcagggagtacagactacatgagaggggccgcagtgatgtaacagtctgggcgtcttgtcttgttcccgaggaagctgacggggacagcaaggatgcacacacctctttttcaggaggctgtccctgccccccgccccgcccccgctataaaacacgctttttgttggcctctcgtggtcctcaccaaggggatccagcttgtatctcctgtaaggtattgatgaccccgttttacaatggggggtggggacaaggtttgatttgtcctccacctcttctgcacacactcaagtggacagagaaccctggccatggggcagattgaccagctgggtggtgggatgccggacctcaggagcagttggtagtgggagatgaggcccctctgctggaagtctgaacccttgagcctgagaagctctgggaattccttgtctctctggaaatacaaatatcagtgtgggaagaaaatgaccaaagaacgtgattaaaggtgactccaaaggagatgttctctggagaacacgaggcctgaaaatgaatccatgtcactgagttaatgtaacggaggcaaacatgagatgcaccagccgaccaacatagtgaggggggagggtcacaaagagcaaaaaagccgcaaactcatgaggagcttgtgtggcagaagggccatccacactggtgacagggagataacagcagggcacttgcaccctgtttgagggttttatatctcttcttttgtctccccctacctctcagttcccttttcggctttagggcccccggcaagcctcgtgtcctgcacaagaatgaagagaaggagccactgttgcaggtggattcttttgtggggaagcactggtttggtgtgtcatcctggtttaacactggtctctttcccacatacacagagtcaatccccacctgcttcactgagcctgagcttctgaattggcatggtgaaactttccctggctgacatgggccagactctccgtcacattcaaattcatattctttcaacttatgtcgattttcaagcatgtgggctaagaaactggatgagatctggtcctctgccttcaagattaccctgctcttgggttgtaagtattgacagtttctaatgtgtttgccatgacatttcagtggttgtatttctttgtataagcatcccatctccctcttggcgatttatattaacagttttgaaaaagcccagactaattgaggaggtttgagtcccagtgtctcacaagcaccctccagaaacagtctgttagttctggagggtaagaggaagtccctgtggcgttgtggcaattttcagacatccctagagcccaccagtggtcacggtgagatgggcagatgcttctgacacaagacaacggttctcaggatcctggctggtggcgcccctgaagcaaaggccagagacagacaactaggacaccatctttgtcttgtatgatacacatgactgcttcacccatcactggccaaggagatgcagcaggtcagacatcccagaggcttttgcaatagtgaattgcatgagccatagaacttggtaggaacatctccatgaacccatatctgaggccctttgtccacggggactgtgctgctaagaaaactggcagggagtgggacgggggtggactcttttggcagttagaaccatcctactgtcctgaagtcaccttgaaacagtgggtaatatcctgggccaggtccaagtggtggtccaccaagcaggaccttgtaatcaggtggtatgcactaagtgggcttccctggtggttcagtggctcagtctcataaagagtctacctcccaagcagcagacatgggttcaatccctgatcctggaagattccctggaggaggaaatggcaacccactggagtatacttgcccaggaaatcctatggatagaggagcctggtgggctacagtccctggggtcgcaaaagagtcagacatgacttagcaactaaacaacaagaaatgcattaaataacaatcttttatagtgctcatctctctcccctgggtcctcatttcccaccatttagttacccttcgatgaaagtggaatgggaagaatgaactgtaaaaaaatcaccaatttgctcttcctcttgagggcaggcccccaagggaatgacaggtgaggagatagcaagaaggatcttgggaagaagggcataagatgaagtcactggtacctggacagcgttatctagtgggatctggagagggaagtggaggggaaggagtcacatttataaagcagcaaatgccaagctcagcacctcatttattccattaaatcttcacagcagctccctgaaggtggtattaggggctccatcttaaagaggatggattaactcagagatgaaggtgcgatcagatgtgttaagtcaactgccaaggtccatgaggctggaaatggccctaccccaaactgacccagctgtccactcagaccagttggttctcccaattttacaggggtgacttcactttcacttttcactttcatgcattggtgaaggcaatgactacccactccagtgttcttgcctggagaatcccagggacaggggagcctggtgggctgccatctatggggtcgcacagagtcggacacgactgaagcgatttagcagcagcagcagctgcagcagactcacccaaggagcttagatatcatgtggattccagtccttcatgccagagatcctgatctctggtactcaggacattttggaaactccctaagtgcttctgaagcagctggtttgcagagcccatgcaggaagcaccacatcttcctttgggataccagggagagtcaacacggggatctttcagcaaggaaagcccaagccccctttgagagatatgctcaatttctctttctcctgcatccagacctcggtgctcttaacccactgcccttcaactcaaattgcttccctctcggtgcttgttttgagtagggacatagtctaactatataggattggcagttctgttcagggctaaaatgtgaagacagtttgggcaggcacaaaccgaacagatttgagtagtgacagagacaggtggcgaagtgattcgaagtcctgaggatcctgaccagtagagttgctgcttgtggtcacggtttcaaggatgtcttgactgtcagtttcctagtgtctgactgaattagatgctgactgtggttgtacatatgcggtgcagagcgccacctagtggaacttcatgtgtgttgctggtctgcttctgagacccttgctgctaagtcacttcagtcgtgtccgaggctgtgcgaccccatagacggcagcccaccaggctcccccgtccctgggattctccaggcaagaacacactggagtgggttgccatttccttctccaatgcatgaaagtgaaaagtgaaagtgaagtcgctcagtcgtgtctgattcttagcgaccccatgaactgcagccttccaggctcctccgtccatgggattttccaggcaagagtactggagtggggtcctaggtccaaaagtggggtgcggacagagaatataatagatataatcatcttttagaaagtgtgtctgtggctattttatcatatgtccagtgtgttaacacagaaatatgtagggctgtatgttcaatgttgtttattaatgagagggcaatataaaaatattgaggaccatagactattaactctttgtttcaaccacttatttccattgcttggcttgttgtctcaagaaggggctggccatgcattgccatttgcacatgtgggtgtagatgaaaaacctgagttaactgggtgaggggctttcttttccctatgacacatctcctctttccatagaacgtggccgtcatgttgaactgtggtttatgtagtagtgacaccctttagtcgctctcttgactccagtctcactcactaagcactctctctctctctctgtctcctatttctgactaatctacctgttctcctcctccacctcctcatcaccccccttagcccatgaatggcatctttaaagcctgctgtaatttaattgtttgaggattagcggtctttcccatcctacttaactgtgagttgatgaaaactgtctctgggtgggcatgatgcttgggggtttcatgaccacctcccacaggtagcattaaacaagtcaaagatgatggcatcctcacttgctcttatatatatagaacattcctcatattcagtaattcattcctcatattcactaatatttttaatctttggctcttagtgtacagtatctggaaatttccttcttctggccttttctgctgtataaaatttatgtatttcatgtattatattcatataattaattttttttatgcaaagggtgatatttcatttgttagcgcttccacgcattacatagtcactcattgtttggtctcctcttgctttctttgaattgaagtccatgagactttccagaaatgtctcagatacccagagctcattttttaggccttttttgtaagtataagttttcattttccataatcttcgtaccatgagaatgctgaccacttcctgctcaaaacttatcagaggaaagtcccttttcgttgatccaatttcccatatgtcaccagttctgctttttcctatgatcctacaagaagatgaagaaaatccacaaatctgaaggagctccatgttcatctacatagaagcctgtgtgcttctttactgtcaaaaatcaggatgcagtttttcctgttcttcccctcccccaccatctcctggtattttcactgactctgaatataatgggttttttccattgtcacatatgagtttacaattttcctgatatcacctgttttccaaaaacttcttttaaaaagccttgtggagagagaccgtatcaaggtttacatacaaaagcataaagtgctctcttctgagaatgtgatgggcacggttgatctctccctaagaggtgctagataccaaacctgcatcaagtcccaccttggtgaggttcacttggatgtttaggattccatttagaacgtgaagtttgcagaattgttggggtctgggggagtcgagttccttactcttctctcctgctgagctatacattctggcgtcaggtggggttaagacccagcatttgctaaatttgatgcccatggcatagacttgtcatctgacacctctccaaccccccaactccatggagttggcccacccagctacaggcagaaaagaagaaacttcaatccagagtgcaggtgccctgttatacagggcttacctcagggtggtagaagcaaggctgtggggggtgtgccgttctattggcaggctgttctgagagaacaagagccaggggactgagacaggcaggagcagttaccattttccagctatcatcaatcagcccatagcagaccacatgccccactttaggacttgatgtcactgagtgaagagatcttgttgttagagctcccaagaatagcaaggagagataagaaagccttcctcagcgaccaatgcaaagaaatagaatgaaaaagactagagatctcttcaagaaaatgagaggtaccaagggaacatttcattcaaagatgggcacaatacaggacagaaatggtatggacctaacagaagcagaagatattaagaagaggtggcaagaatacacaaagaactgtacaaaaaagatcttcacgacccagataatcatgatggtgtgatcactcacctagagccagacatcctggaatgtgaagtcaagtgggccttagaaagcatcactacaaacaaagctagtggaggtgatggaattccagtggagctatttcaaatcctgaaagatgatgttgtgaaagtgctgcactcaatttaccagcaaatttggaaaacccagcagtggccaccgaagtggaaaagatcggttttcattccaatccctaagaagggcaatgccaaagaatgttcaaactaccacacaactgcactcatctcacacgctagcaaagtaatgttcagaattctctaagccagacttcaacagtacacgaaccatgaacttccagatgttcaagctggatttagaaaaggcagaggaaccagagatcaaattgccagcatctgttggatcatggaataagtaagagaattccagaaaaacatgtacttctgctttattgattacgccaaagcctttgactgtgtgtatcacaacaaactggaaaattctgaaagagatgggaatacaagaccaccttacctgcctcctgagaaatctgtatgcaggtcaagaagcaacagttagaaccagacatggaacaaccgactggttccaaatcgggaaaggagtacatcaaggctgtatattgtccccctgcttatttagcttatatgcagagtacatcatgcaaaatgccgggctggatgaagcacaagctggaatcaagattgccaagagaaatatcaataacctcagatacgcagatgacaccacccttatggcagaaagtgaagaggaactaaaaagcctcttgatgaaagtgaaagaggaaagtgaaaaagctggcttaaaactcagcattcaaaaaacaaagatcatggcatccggtcccatcgcttcatggcaaacagatggggaaacaatggaaacagtgacagactttattttcttgggctccaaaatcactgcagatggtgattgcagccatgaaattaaaaggtacttgcttcttggaagaaaagctatgaccaacctagacagcatattaaaaagcagagacattactctgtcaacaaagttctgtctagtcaaagctatggtttttccagtggtcatgtatggatgtgagagttggactataaagaaagctgagtgccaaagaattgatgcttttgaactgcggtgttggagaagactcttgagagtcccttggactgcaaggagatccaaccagtcaatcctaaaggaaatcaatcctgaatgttcattggaaggactgatgctgaagctgaaactccaatactttggccacctgatgcaaagaactaaatcattgataaagaccctgatgctgggaaggattgagggcgggaggagaaggggatgacagaggatgagatggttggatggcatcaccaactcgatggacatgagtttgagcaagctctgggagttggtagttgacagggaggcctggcatgctgcagcccatggggtcccaaagagtcagacatgactgagtgactgaagtgaactgaacacctgaaactagcacaacattgtaaatcaactatattcaattaaagaaatttttttagtgtaagtagaagaggaaactccctccagagtatcattctgaatcaataagacatgaataaaatctcaattaaatgtactatattaataaattagcttctacaacatcaggccatgcttggacgcatgaaatgaagaagactcttctttttagagtctaagaccaaaacttaaagatttctcatcctttccggagacagacagagttgttaccttcaagggcactaaagatatcatttctaagattccttattattgattcaattcagttcagtcactcagtcgtgtattattgattgggtgccataatttccttttaagcatttatgaaatagtgccgaataagttatttagaatttgtattctgcattatattgatgtatttagaagctgtcaattatattcactcactcactcattcatttattgattttattcaggtagtcaataaatattattatggaccaggcatcattctagggcccaaggagacgctgatcagcaaaaatagaaatatttcatttcttcagagcttattcagaaactcattactcagataatcatattgggggagatttaagtactttgaaggaaaggaagaatgttctagaagattatataattctcacattacaggtcagagtaatgagtcagattggggaactggaggagcaggcagcctaaggaagaccaccagagctgagatgtgaaaatgagttgactgaacgagacagcattgaaagaggacagcatttggaggtaggagatcacaagtgtggcttagtaatattgatctggggaggtttccagtacccacattggagatgctgagtcagtagttgaccatcatctatggagctcaagaaagagagctgtgaactagagacacgtagttcaggtctcctgtggtgatgacagtgacggacgccttgcatatcaatgatataatttagggcctttctatccaaactgtggtccccggaccagccacattggcacctcctggaagctccttagaaaagcccgctttgtacctgctaaatcagaatctgaattcaacaacagacccaggcggttgccaggcatgctacagtgtgagaagcactgttctagggtagagcttattaacctcagcattaccgacaaaaaggcctttgttgggatgaagtgtgtgggggggatgcttttgaagggagagctgtcctgtgcattgtagggtgtttagcagcatccccagcctctacccactagataccatgagcacctctcctcccctcaagctgtgaaaaccatccatccctgccaaatgtcctttgggggacaaaatacacctcaatggagaaccactgacctaagggcaatgtttggagtagatgcaaagatgatgggaattgagctttgaagaattctaattgttcacagctggcagaagatgagcctggaaaggacagtgaaaaggaggagacagagaagaaaaatcaggagagcagtatccaagaaatcaaggaagtgagggtgataaggacgatgtcacatgatgctgaaatgaagtggaagatgaggactgcggagtgcccatttaattgagagagggagagagaaagagagagagagagagagagagagagagagatggaagtgaccctctgggatttgttagaaatacagagtctcaggtcccaacccagacctcttgaatcatgtccccgggtgattcctgcacattaaagtcgactaacaggcaagtcttcagtctgatagtcaaagagaagaaaaagacattgtgcaattttatctacaaccggtgtgctacacatgactgccttaaatatacttatcgtctataaatagatttaaaggaaccacatgtggttgaacatggcacatgtttaaacaggaacatatgcaatttccaaactcataaatcagagctctggttgtactcctattttcaagatagatggtggcttcaaaatttcatgattttaagtctcaagtacagttgttactttatgcaggcaattagttcccaataaaaaatgaacatgtgttttgcacctaattgactttcttaaacaaagactatttagtatagtttggtgtaaacagaatcttaatgtttttactaattgcatttgtatcagtggatgtgcagttgacgtctattactctagactggtgggttttttttcttcctttttctttttctccctgattttattgagacagaattggcaaataaaaattgtatatatttaaggtctgcaatgtggtggtttaatatatgcatatctagtgaaatgattaccacatatccaccacctgacatagctaccattttgttggggcgggtgtggtgagaacatttaacatctactctcagcaaatttccagtgtagaatacagtattagtaatgtaataatcgtgccttgcatttgatcctcagcttattcatcttataactggaagtttgtaccctttgaccaacgtctcccatctgccccagccaccttattctagcccctagtaagcaccattctactccctgagtctatgagtttgactatttaatttttatattaaaaataaaatttttaaaattattttttaattagtcaaaatttttaatagtcaaaaattaattgactattttctttttataacttatttatgtattttgtggctgtgctgactctttgttgctgtacgcgggctttctctagtcgtgctgcgagcttctcttcacactggcttctcttgttatggagcacaggttctaggcatgcgggcttcagtagttatggcacatgggtttagttgccctgcagaatgtgggatcttggttcctaggccggggatcaaatccacgtcccctgcattggtaggcagactctctctctttctctcttttttaaaaatataaaatgtctctaataattttatactgtcatacatttttacataatatttttgagatactgggttagcctgtggttatgttagtcactaagttgtgtccaactctttgtgacctggtggactgtagcccactaggctcctctgtccatgggatttcccaggcaagaatacaggagtgggttgccatttccttctctaggggatcttcccagtctggggataaaacccacatctcttgcgttacaggtgaattctttattgctgtgccaccagggaagcccatattgggttatatatgttattttaatgaattgtaattgtggctgctaggaaatttaaattacatatgtgactcatttttgttggacattgcagctctaaaagctgaagagccaatgggtatttgctgattttcatggtgaaaaagtggcttattcttgttggtgtttgacagaaaacaacaaaattctgtaaagcaattatccttcaattaaaaaataaataaattttaaaaagaccttccccccagaagtgaactatttaatttctctgttgaaaaggaacagaccgattcttattgattcagaatgacactctggagggagtctcctcttctgcttacacttaaaacatttttttcactgaaaatagttgatttacaatgttgtgctagtttcaggtgtacagcactgctatatataaaatagataactgaccgagtcttaatcactggaccaccagagaagtcccgagtttgactattttgctgctgctaagtcccttcagtcgtgtccgactctgtgcgaccccatagatggcagcccaccaggctctgccatccctgggattctccaggcaagaacactggagtgggttgccattgccttctccactgtgtgaaagtgaaaagtgaaagtgaagtcgctcagtcgtgtccgactcttcgagaccccatggactgcagcctaccaggctcctccatccctgggattttctaggcaagagtactggagtggcttgccattgccttctccgtgactattttagattctacttataaagacatcggacagtatttgttttactctctttgacttatttcacttagcataatgtccttcacattcatccatgttgttccaaatggtagaatttcctttatttttatggctgatatatatatatatatatatatatatatatatatatatatatatatatatatctcacatttctttctccatccatacatcagtggacacttaccctccctgctgggatagcctgtttctgtatcttggctaaagtaaataaggctgcagtgaacacagggcggtgggggggcagacacctgtccaaattagtgttttcattttattcagataaatacccaaaactggaattactggatcatatggtataattttttgaggtacctccatactgttttctatagtggtcgccccaatttacatctccattaacagtgcacaagtgttccttgttgtctacatccttaccagcatttgttatttcttggtttttttttttttttttttgatgatagtcattctaactggtgtgaggggatatctcattgtggttttggtttgtatttcccttttcatgtacctgttggcaattagtatgttttctttgggaagatgcctgttcagatcctctgtccattttaaaattgattgcttgctttttgccattgagtaatataggttcgttatgtattttgaatagtaacccctaattggatatatggtttgcaaacactctctcctactttatagatggccttttcattttgttgatgtgtttctttgctgtgcagaagcttttcagtttgatgtagtcctacttgctgattcttgcttctgtggtttgtgcttctggtgtcatatctaaaaagacattgccaagatcagtgttaaggaggtgtttcctgttttcctcgaggagttttatggtgttaggtcctgcatttaactctttaatccatgcaaacacacaggtgtaaggtgaccctattccatccatgataaggactacagagagtctcaggctggggacaataccaggctctcccagcagggagggttcatttattctaagacagtcctggtgaggcctcagtttccccacttcctaaaagtggttcttaaccttggctgcccttgaaattatgtgaaagtcgctcagttgtgtccgactctttgcgaccccatggactatacagttcatggagttctccaggccagaatactggagtgggtagcagttcatggagttttccaggggatcttcccaacccggggatcgaacccaggtctcccacattgcaggcagattccttaccagctgagtcacaagggaagccccaaaattacgtgaggttctttaaaaatctccatccatgaatatactgatttaattggtctgggatagagcgtttggatttaaaagcttgcaggtgattccaatttgcaggcaacagtatgaaccattgctttagaatctttgctgtattgcagttaggtctgagacctgagcatgcacagagtcagctgaggaggcttgttaaacttcagaaggctgggtcccacttcagtttctgattcagtaggtctcgagttggagcctgcaaacttgcatcttctaacaagctgggctgggggctccaaagctgctcggctaagaatcacacttggagaactcctgctctattgagtagtgctcaaacttcgttggccccagagctcctgagttttttgagtcctgacatttgcattttaaataaaaaaacagctgattctatgcacgtgctgtagagagaaacctttgcaaaaccctgccctggatgctaagatggcactgggaggcagagtcatctttaagaatgacatctgtcttcttcccttttcatcattctgattcaagttggccttaagctctgtgcccactgccttgggggtcttgggtggggtgagtacgttatttggattttgttatttatttattgggcagcaccgggtcttagttgtggtgtttgggacctttagtctcagctggtgggatcgagttcgagcccatgccgcctgccttgggagcctggaatcttaaacctggactacaagggaagtccctcaaccgtttttgttttttttgttttttttttttgggagagagaagccaagagggaagggaattggagggctgagaagtccgattgattccgagaaaagagaggacctgccaactttctgtggaaaatgcttaagaaatagattgtgcattctctcacatgcatcccgcacccccaggtgttactatattagactcagaagctgcgtacaagtcctggaaggcaaggctcctccggtcccctgaaggtctgagttgaccttcgggagacctcaggtagaatgaaagctcagggacagaaacaaaatcaaatcataggaaatgaagctgtcttgctgtgcatcccagttggtagactgacacccacaccgggtgcattagctctccactgtactcctggaacgttttctacctggctggctttcttttgtaatctggttttgttttgtttactcaaaatctaactcagtaagtggattgagtattgttgttgttaagtcgctcagttgtgtccaattttttacgatcccatgggcttccccgtccttcactatctcctggagtgtccgagctctatttcattcagtttcttttttttttaagtgtagtcatttggctgtactgggtcttagttcttcattgtagcatgcaggatcttttagctgcagcttctgaactcttggctgtggcacgtggaatctagttcctttgactggggatcgaacccaggcccctgcattgggagtgcagagtcttaagccagtggaccagcagggaaatccacatacactttcttcatttatgaaattgacccttctctagatcattgtttagttaaaagtctctatctgttcccagatggaggggcaaaagcttattgcttcatggtgtttgttttccttgaatcttaaacctttgagacccatagggcaaattgtgcataaacattgtttttatttggaaactcgagttgcgttaactgaaatggcccccacatgaggaataagccttgtatacatgtgtagattgtagtaacctttgcagtaaaattttatactcattttatcccatacacatcacacaaaggagaaggcaatggcaccccactccagtactcttgcctggcaaatcccatggatggaggagcctggtagtctgcagtccatggggttgctaagagtcgggcacaactgagcgacttcactttcacttttcactttcatgcattggagaaggaaatggcaacccactccagtgttcttgcctggtgaatcccagcgacaggggagcctggtgggctgccgtctctggggtcacacagagtcggacacgcctgaagcgacttagcagcagcagtagcagtagcagcatcacacaagaagaacccctctgcctatagtcacaagtccaaaatactgggtggggagcgccacctggtggcaggatggttattagctcctttgccatgggctggatcttccagaagtaatttctgaccctggaggggtactgaagtcctttgaggggctgggaggggagtgtggagtgcagacccagaaaagaagaaaccaaaatggttttatacaacactagaagcaccatgttgagagaagtgaagattgggaaaagagaattagaaggcaagaggaaagaagacaagttggggtgaagagagagaagaaagagttctgaggattgtgggaaatccattgatgacctggagtcttcctctctgcacccagcccggggcctcaaacctaagagcatgttcttatgtgttcagtgtgccctcatctgcgtgaattacgtggctgtcctctgagtcttctcacatgttctcagcctctagtactgacacgtgcccctctaccagctgcgctctctcctggtaaccaacctcacctcctatatccatcacagagaaattggaggcccccaacatgcacttcttcaaatctcctcccctcaacatcaaaatatgttttacgtgactgtcctccacaccagctcacaaaaatgagatcctttccccaaagagcccgtcatgtttgcaaggagtggtggcccctctttaggtggcccccccttaggtggcccttcctaagttacagctccacccacctctgctcctggttcactccctttggcctacttgctgtatcagatatgcttcatcatccctcttgtccatcctgctggcacttcccctcacttgaacatagttcttctaaaatgaccttccctccaaaggtgtttattgtggcattgtttgtaatgttaaaaaatcagggcaatacttcattcttttttatggcacagtaatattccattgtgtatatatacacatacgcatatgcatacaatggaacatgtgtgtgtgtgtatacacaccacatcttcttaaaccagtcatctgttgatgggtgcttgggttaccttcttgtcttggctattttaaataaaccacctgaagctaacatagtattataagtcatcaatacttacagctcaggggaaaaaaatgattaggacacccaagagtcaatcaataggagattcattaaataacatgttatgtccatacagtgtatacagcatagccattaagaaacaaaacctggaacttccctggtagtccagtggttaggactccatgctttcactgccaagggcatgggttcactcactggttggggaactaagatcccacaaaccacacagcacagccgaaaaacaaaaaccccaaaacccagcccttgggttcagaagatttaccgtctgatagaggaggtaaaatggacctaccaaaatacaatgcaagacagaacgtgacaagttctacagagctttcggttcattggggcgaattactggagcttcgcaagcatgcttctgtctctaaaagtgcagattttaccaaccaccaaagcacagtgtgaaatgtacatggctagagcctcagtactcttgcctggaaaatcccatggatggaggagggaccccagatggggtcgctgggagtcagaaacgactgattccctttcacttttcacttttctgcattggagaaggaaatggcaacccactccagtgttcttgcctggagaatcccagggatgggggagcctggtgggctgccgtctctggggtcgcacagagtcagacacgactgaagcgacttagcagcagcagcagagccacaggtgggtgcctgggacagaggagattaaatgccaccagaggtagggaggggcctgggaactgaacacagggcacacgtctgctgggtgggcctggccacgtgggtgctggctgagatgagcaggctgctgacagctgtgccccaaacgcctctagctaggaggcacccatcccaggaggtgggggccagcgcagagctcaccatgaagcaggaagaaggctgcagaggcaagcagggtgtggggccttctcaagcagtgaggggcagtcatgtcaacctgaggaggactggggtccaaggctatggagagggaacattgggaaagactggatcaagattgcagagaaatttgcttctcagctcaggggttccacctccaccatgatggggatcatcagtgtgctccttctggaaagttcctctggcgtgctggggttggagctggaaagtgaccatgtttgttgtcatccctcactacttgtttagccctttagaacccagcgtttgattctaccacattggaagctactttgttgagtgtcaccaacaatcttctctttgccaaattagatggcttttatgagtctctgggatcctgtgcgtggttaggatattggatagtgggtaagagcacaattctggagccagaggaacatgactttgtttcctcactgtgctgggtcttcatcgctttgtgtgggctttcccgagttgtgatgagcaggggcttatattgcagtggcttctcttgttggagagtatgggctctagaacaccagctttaataactgtggcacacaggcttagtcactctgtgacatgtggaatcttcctggagcagggatcaaacccgtaaccctatacattggcaggtggattcttatgcactggaccaccagggaagtccagaatacgacacttttcttaacctctttgtgcttcagtttcttcataggaaaactggaaattgtcacagtccctccatcatggggtttctgggcaaggtaaagaaattcatcaaggaaaaattccaggcccatagtaagccctgcctaagtcagctttggctatgaagcacatcctttcttaggaggttaacaaagggagaagttgaaagaacacaggagcagaagtggagccgcaagagttctctccagcactgttgcactcttgacctttggcttcagtttcctcatccatgaaataaggggcccttccagctgtagggtgctatgggtttttctgatgtcttggccaattatatgggggacactgactttaggaaagacaggaagaggtttcaattgatggaaagactgagattgtcaggtaattgggctgcatgcaacctgtgcctgactcaagaagggaatttgccaagaacaacagagaccttcccttgtccccattcttggtaagtcaggtgtgctgttgaggggagatggtcccattcagtatgatgcaatgagttgcatttagctgggcagccatgtaaacttgcataggttgtccactgctcatatggccaagcaagcctggaatccagccctccctcctcttattcagctctgcgccctgatgtcgggtacatcagcccacaggaagcatgctctcagcaaactttggtaactgtggcacacaggctcagttgctccatgacacgtagaatcttcctggaccagggattgaacccataaccactacattggcagatggcttcttgtgctgtcattttctaattggcactgaggagttatctagactagttttaagctttcgtggccagacctggagtggtcctccaggcctacttcaggcagcagtcagaggcgagtgaagtccatgacaatgagagaagtaataatctgctccacttctggagtgcttactctgtgctagggactgtactgagggttctgtaagcatgatttaatcctcacagcccctgttcaaggatactgttattcttctccccatttgacagatgagaacactgaggctcagagaggctcaggtgccctggatgggggtttgtgcatctggaatatgcatgctgcctggtgggttttcctggtgggagcacttgatcgcctccattgccaaataaagatccgagtctgcatgcatggcgaggggaagagcaagggaagagctgcctgaagctctgggctctcccagggtggggagagcggacctgggccctgaaagctggtttctgggcttgggcagagggctgggtccttaaggccactttccacgtgggatcaacttctggctcatggtgactgttttgacctggtgttgattccatttgcatgctcatcttttttggttactggctgtgtggaaaaacctgtgatggctcatttgatttgaataagtagctcatttttctgatctgcctggcgtgggcactggactgttggcaagtataacgcgagtgtgtgccatgctttgtgtgatcccagctgcctggtgggtgatttctgcagcctggggccaggccacatgtgtctggctttttctcaaaaggctgagcatgctccactgctctactactggcagtcacactgacttactttcttaaactatttctaagcaaatgttttaagggaaaccaagagactgggtggggcttccctggtggctcagtggtaaagaacccacctgcaatgcaggagacacgggtttgatccctgagtcaggaagatcccctggaggaggaaatggcaacccacttcagtattcttgcatgggaaaccccatggacagaggaacttggtgggcgtacagtccacgggattgcaaagagccggacacgactgagtgcacacacactcacacacacacgcaagaggactgggtggtggccagatgaggccttcatgttgattataacccagagttttggtgattccatgaagccacgggtcgtgttctctgatatcataggaatatctatgctatctttctcacgccatgtgtcaggccacgaatcacctgagagaccggtggagggcagttcaagagttgctgccaagcctgcgtgttctgaaaggcatgcctttgtaatgaggttgtcatctggaagtcatgcacaagaggcagaccatgggcctgagggaaaataataatggtgaagtgtgtgtgtgtgtgagaaaatgtaaccccctacccactctgccccccaagtggctccctccaattcaaggagagtcagggaaatgaaccacattatatcccttttgagactattcttttggccaggggtcagcagatattttctgtaaagtaaatattttaggctttgcaggacaataggccaattgagggcattatgtaggttcttcagagaaggcagtggcaacccactccagtactgttgcctggaaaatcccatggatggaggagcctggtaggctacagtccatgaggtcgctaagagttggacacagctgagcgacttcactttcacttttcactttcacttttcactttcatgcattggagaaggaaatggcaacccactccagtgttcttgcctggagagggaagcctggaggggagcctggtgggctgctgtctatggggtcgcacagggtcggacacgactaaagcaacttagcagcagcagcagcatgtaggttctttaagaaaagaggaattttataaatattaatcaaatgcagggttttttttataagtctattaataagaatggaactctctggaattcttttgcctcatgggagtttatagatagtgttctttctcaaccaatcaatggcaaaggttcatctgtgaaaattgttctctactccctgcccaggtttggcccacatgctggagtttgctgactcctgctcttggtaaaccttgtacagccttctctctgagaccttaagaagcaatccacttatcagcagatcaggaagtaaaattttcctgcctcatgctcctgctcatcccttcagtcattcaccaaaggtgtattgagttatgctacatgctagggactgtctggggttcaaggacacccaggaggaagatggtggggggacccgtgcccaccacaccaagggctgtcccagagatgggcagaacaaagatgaagccattcaaatgttatttaactgcatccatgaattccatgagtaaaatgagtttgtgcaaccataaatcaagtatacatttgtggtaaagttgggaaaaaacaccctacatttttttaaggaaaaatttccccatttctccctcctttccccctcatcctgctccccagagctcactgctcttaacatttcgctggggatccttgcagcaactgcccaccaacccacctggacaaacgaacccaaactcacaaagatgtgccaggtcgatctggcttgggttatatttagatcattttataaatgccttttttccaccccctccacttcagttttgaaattgtacagcctagtcaataaatgatttggcaacagttaagaaatcacgagttccattcttcccatataagcttctgcaaaccgtaggccaggctctactgctggtaaagcggccagttttaggaagttggttagtttctttgtgcgcatacacccaacagcacagaggttgccactcggcagcaccgcaggttctagaatgagcagttaggacaccaccagaggcgcggtaagccccttctattcttttaacaaagctttccagcaaaatagaggatttctggccatgaaacagggtttggtggcaaataactgtgaccaaagaagtggtgaacgggcagatgagcatgaaaaggcgaagtcttggagcagctctaagtgtaggtggagtggtctggtgtgcaggaagtgtcagaaatccaatgccgtggtcttagcattttcaagaggaaaagaagtgaattccaagttaggaattgatccggtcctgccagaaatagtattctcatctagagccaagaggctggaggggaggtgtttgttctttgctacttctacagaaaaggacaagctgtctttgctcactcaggctgcttgctggtgcctgggcagaacctggacttcatctcctaccaggagcttcaggtgcattcagtatttccaggctttcttgttcagagtgttcaagtgaagatgttactgttgctataattatcttctgtaatgtttagacatagaaggtggtaagtttttgttactgtcaagaactgtcaggttgaaaaacttaacgattttttaaaaatcatgtattttctcgataattgaatgcgatttaagcaccattgcaaagactgatttccagagaatctcatccaatgtttactttagtatatttttttccataatcatttagtgcctattaaagttcttggagttattttttttttaccactatttaacatatgtaagttcttgaagttcatttttttatatccagtgactgcccattatagatcttgaaagtaaattaagtctgctctgaaattctctgatgctttgcagaatgactgcaaaggctcagaaggaattaggccagcagggccaatgatatatagggaattgcatttaaacttatcccatttctacttgatatttgcaaactgttctgatagccaatataattgcattataagaaaatgatactagaacaaaatcaaactcaacttggaaaaatgccctgcctccctgtcattggtggtgggtagaaggcttgctgactcctggtctcaagagtacaggttcttaggcttgactatgtgtcagaaccacagagtgaactttaaaaaatccccctgtccagactgaaccacagactaatagaatcagcatctctggagtgggcctgggcattggtagcttttcaacttcccaagtatctctgcgagcagctagagtcaaggaccaccgccttagagaagcatctgaacatcccatttggtcttttccacaaggagcacagacttttgggagtgggactgacaaagcctccagaaagacctgaagcttattcattagcctctcagcttgattccagaaagcaaaataaaagttccacgtataccttatccaaaaagctgaggctgtggatcagggcagggacacttgatattgtccactcttctttctgcttctcattcttccatgtaataatcataaaaagtgcacaggaattgatgagagagaagtcagtcctgtggaaacccagactgacagagtatcagggtatgtctgacatgggagggtgtcacgacccagggtcagccttggaaactcagttggatattatttgtaagtgcgtctcagcgtcacctctggggtctattgagggggatgtcattccagggaaatatcctttgagttcagctgacctattgaacagtgctgagggatttgttgttttttaaattgttactcccaatttataactcttcagggctatagagacagtacatgtaaatgcataacccatggttctgggtcaacgttacctagcacagactgggttgagttttcagctgattgttaatagccactcattggctaatatgtctagaatcaatggatttccagttgttctatggaattacagagtacctgtaagtgctctgtggcatcagtggaaaggcaaggtgggtaatactcccagccatttaagtccagaaggttccatgtatggctgtggtgtttctgctgataactgaaaccagatcatcgcttggtaccaccttgttacctatgtgggtacctttagactcagtttccgttctgccacctaccatctgattgaccttgggtaaatgagcctctgagtctcaggctccatgtctgtaaaatgagggcttatgacagcacccacctcactgatttttatgaagatcagacaagaaaatacatgatttgtgctcccagtgtttgcctcatgataaagcactcaacaaacgtgtgctaccatatttgctaaatttcaaatagcataatttttcatatttttatgtgtatgaaatcaggctccacctcttcgttgatacgtgcatttaatgtggtagtggtttctttggtcttccaaaggtgttagtgaatccatcaaatgtggaatggatcaagtcaagtagctagtatttactgtaaatttcatctcaggtgacaaattctctctgccacttctttatcaatttactttcataatgtatcagtgctcaacatagtcaactatgaagacatcagaacccatgcaaaattagaaaacgtttccagtttggcaagcatttagttccagtggaatcatgcatcatggccatcagctcagtgattcagttgggcggattcatcatctttaacaacccataggttcctttgacacttttgtgtcagggggcatcagcaggatccattctgagaaagcagtgcagctcaggagatggcatgaggcagatcacatcttagcaaaggaggctttcagagagagaggtgaagtagtcctactctttgtgacttcatggactgtagcctgccaggctcctctatccatgggattctccaggcaagaacactggagtggcttgccatttccttctccaggggatcttcccaacccagaaattgaacccgggtctcctgaactgcaggcagattctttaccgactgagctatgagggaaaaagaggactggattatcatacaccatggacccagccagagaactgatttgctgtggaaatcaggaaaacagttgtgaaaagactgcctcaatagtgagcaattttttgaaatttattcagcatggagcaggatgtgaggcaaatttcttttgttgtaaaatcattacagatcatgactgcagccatgaaattaaaagatgcttactccttggaaggaaacttatgaccaacctagatagcatattgaaaagcagagacattactttgccaacaaaggttcatctagtcaaggctatggtttttcctgtggtcaggtatgaatgtgagagttggactgtgaagaaggctgagcgccgaagaattgatgcttttgaactgtggtgttggagaagactcctgagactcccttggacagcaaggagatccaaccagtccattctgaagaagatcagccctgggatttctttggaaggaatgatactaaagctgaaactccagtactttggccacctcatgcaaagagttgactcattggaaaagactctgatgctgggagggattgtgggcaggaggagaaggggatgacagaggatgagatggctggatgtcatcactgactcgatggatgtgagtctgggtgaactccaggagttggtgatggacagggaggcctggcgtgctgcaattcatggggtcgcaaagacttggacgtgactgagcgactgatctgatctgattcatatgcttctttaaagaaatgggttctcgatttttttttcccattggatattccaaagcaacacgcagggagaccgtgtagactggaccatcgttggcagggtgtgctatgcagagagctcactgccatggccaaagagctgtatctagcagatcgaacttggctcagccatggggtccactggtcagcacttttccccctgctctctgctcttcctctctgtttcctggggctgccaccaactgggtgactcaaaacaacagaaatgtgtcctctaccaattggaggctgtgagggacagtctgggctgtgccttttccccagtgtctggtggtgcctggcagtccttgatgttccttggcctatagacatatcactctggtctctgtctccatctccacatggctctcttcccttgtctgtgtccaaatttctctcttcttatcagaatagcagtcactgggtgagggcccatttgagtctagtatggggcttccctggtggcttggtggtaaggtctgcctaccagtgcaggatacatgatttcgatccctgggtggggaagatcccctggagaaggaaatggtaacccagtccagtatccttgcctgggaaatcccatgggcagaggagcctggcaggctacagtttataagatcgcaaaagagttggatacagcttagcaactgaacaagtctaagtatgacctcaccttagcttgattccaacttcagagaccctatttccaaacatagtcacattcacatggagaagctaaaatgtcagcatatcattttagggtgacacgattcaacctgcaacactgtccttttctggtcctctattctgccacttccactcaaccctaggcctccccttcaacctctcccaacctaatacttaccttacttttaagcaagtatctcctatttattttgactactttcccaggacctggtctgagcccccaggataccatcattgacaaccagcgccagtctctctttggtcagaaaagacacaaggcctgtgacactttaagcaaagactttagttgtttttaaaatctgcagaagagtgaatatattccagtctgggttatatttgtctttatgccaacgtggtcataacatataattttttactattttttttttttttttttatggaggaagaggctggtaaaggccaaagcatctcaggcccacgaagtcaaaactcagtgttgttgacaaccacgttctcttttctgatctgacatatgccaagcacagttcaggaattgctaataggtttccagtgaaggagatttaattattgaatcacaatccattgcctgcaacctataggctagaattgtgtcaaagttcagaatttgcactgtagcgagagaagataattttgttgttcagttgctaagtcatgtctgactctttgcaaccccatggactgcagcactccaaacttccctatccttcaccatctcccagagcttgctcaaactcatgtccattgagttggtgatgccatcatagtacatagaggatttattataaaacactttgtatgggtccacagcagccctgacgataacacatgtggtaatttccactctgaaaccacagagctcccataagtggataaataaaaattttagtagccttagctcagttcagatcagctttttctgtcaaataagttaggaaaaaacccccaaatctggtgctcagatttttaagaatttcagaactgcagagagattattgagcaccggtacaagacaggacctgcattcagaatataaaccatcagccactgtcctggtgaccagaagacctgattctactaattcctttgtctgtgattttcctttttttttttttttgcctatatgcatctctctttgatgctgggtaaatgccaggtgactggtactgactgttacttcgtactcaagcgtggaaccccaaacagctgatgagaagtttcagttgcaaggacaggccttccaaatgatgggtcccagcctaggatgggagcaaacctctgggctgggtgcaccactgtgatcacaggtctcttttgtggtgcagggtcagggctagggtgaggggagtgaggtcctagctttgggtgcagaattgcaggcaggtaaggtccccaacagcttgacagtcaggacacataatattttagtgttctatttttaaaagtcatagtgaggaactctggcccacaggctgggggcctattttggtaaagtaagttttcttggaaccagagctgggatggaggaaggcaggggacttattgcatggcacgtgcagggtccgattctgtcattatttcatagtgatatatggtccatcaggaactttcatttttttgtaaactattttcatcttgatgactaccttttggggcaacctcttaatttttgctactgtggccctgctactgtgcctttccattttcccaatgaggtgctccagtcttctgcttggggggtggagtggagcatttatgaaccaggtggatgttaggggacccagggccagaggagggaagagaacggggatctgcgctccagagacacacagggctttaaccctcctgtttgctttgtgacagttcacccctgccttcacttggtgcctgaattcctgaaccaaggctctggattcaacctttcaaggtagtacgtctcagggagggactcaggcggggtctaacttctcatgcagacttccagcccatccttcgtgcagccccacctggcactccactggtgactcccctgtgactgctgtctgcagctcccgagtcttttgtgggcttgtctgctatgaattgacctcgctctcatcagctttagcccttctccactctgctgtaccacctaccactcatccatctgctttctgtcttcctcaagttcatacacagtccgtgtctgctgtgctttcctcttaatgtcctttctcaccttgtagaatcctaactctttgctctcactttaatgggtaccttcagctattagcctcaaacaacagaagtgtctctctctgtctctccctctctctctcagctttatttgcatagtttggctttctatcctatttgcataattgaccctttcccttctattttcagagttgctatttctctttttttttttttccttcttccacatagttgccatttctctattttatttgcagagttgccatttttattctgccaggctgaatgttttttttttcattatttattattcatttattcactaagggagtaaattctctcgagtacccttcttagttctctgggttctgtttccctttgaggtccttttggagccaagacttttggaagccagaggcatgagtgtgactaagaggaagaacttggctaccgaggtggggcttttgctgcccagggctttgggctctgccatctcacctcagaggccagtggatgttgtgagttagggtttgcgctatggatgatgagcatttgcagtccgttttgcctctctgaggccctgaggtaggtggagaggcttgaactatcgggctttctctttaagatctttgttagatgcctacttcacacctcccactctttccatccatgtacttctctttaaattgaagttatttggaatttgtcaaatcctgtttttacctcaggacactcttttgggaggcaggagttaggaacctcaagctcttcacttagaaatcctctctgcaatattctgcttatttccatggcctagcccctaagttgcctcagattctctgggaggccatggtggtggtgttcagtcactcatttgtgtccgagtctttgtgaccccgtggactccagcaccccaggcttccctgtccatcaccatctttcggagtttgctcaaacttatgtccattgagtctgtgacgccattgaaacatctcttgctctgtcgtgcccttcttctcttgccctcagtctttcccagcatcagggtcttttccaacaagtcggctctttgcatcaggtggccaaaatagtgaagcttcaatattcaggcaagtgtcaaagggctcttcttgtggtctccagcgccacctagaggcttttctcctcaattgctcagtgttgtgtgataattgagtttggaatcagatgttgaggaattaacttgaggctagggaggctgcactggagaaggcaatggcaccccactccggtactcatgcctggaaaatcccatggatggaggagcctgtaggctgcagtccatggggtcgctatgagtcagacatgactgagcgacttcactttcacttttcactttcacgcatttgagaagaaaatggcaacccactccagtgttcttgcctggagaatcccagggacgggggagcttggtgggctgccgtccatggggtcacacagagtcagacactactgaagcgacttaacagcagcagtagcaacagcagggaggctgcacagcaccatcctgtcccggggacacccccaccccaccccacccaagcacccacatagaaatgtctcacctactaacagtactctgggtactgcatgcagggaagagtctgcaaccagcttgccttgccacctgttccccaagagactttttaaaaatatatttttaaattatttattaatctattaacaggaagctgataggcactacctcagccaggtgatcaagctctgcaccaacagtggaagtcatgtatgtgccattgctcagctgtgacttaagtggcctttgacctttgtgattgccctcccccaaacccattaccccagtctaatcctgacaaagacatcagacacgtcccaactgagggacagtctacatgaccatcccctttcccatcattttctccctagtactttccaccatctaacatactgctccaaatggacagatattttcatctgttttgttcattgctatatttcccccaataaagtatatgcttagtaaatatttttgaatgaataaatgaatgtagagttaccttttcctttttaatggctgtatagtattccatttatgactatagcatcctgctttatttaaccagaacacgactcataggcatctagattatttcaaatcttttgctattgctgacggccctgcatggcatatccttgtagacaggcatttgtgtatgtgaagatctctcttgtggtctacttctagaattgaaattgttaagttctaagtatgtctctctaattttgatagatattacaaatttctctccaccttagaggtggccctaatttagacccctccaagtaggaaatggcaacccactccagtattcttgcctggagaatcccatgaatggaggagcctggtgggctacagtccacagggtcgcaaagagtcggacacgactgagcgacttcactttcactttcacttaagtaaaatataggaatacttgattccccacagcctcacctacacagtagaatcagtgaaaaatacctctcctgaaggtctgtgccctcttgccagagactactgtagaataatgagcatggcaccaccctccttgcattgctcctcagatcaagatcaaaatccacactcatggattcttacatccgtgactttctttcagtcccttacagtcacatgctcattcagatgagatttgagaaatgtctgctgatggtagtgacatggaggtgctgaggatctctgtccacattgtctctcacatggccttccagtcctcttggccctacttcttattctgagtcctgctgttgcaagcagttccccacaggtttcagtgggctttgaggatttgaaacctgctgtgctttgtctctggcccacaccattacccctcttgtttgctgtccagggctcctgtggtggcaccatgtgggaaactcaaaggaatgctcgcagggccttttcatatgctgagacattcttttgacatctttctgtcttcttcatgccaaagcaacctgaaaatcccagctcgagtttcacttcctcagggaaacctttcccagcctccatgaagaggtgaaatcctcctgttttaaggctcttctagtctcacttctttgtgtcataactgagttcttgtaatgtggttgtgtttcatctatgtctgtgagttaactaataatctgtgcgtgcgtgccttctaaattgtttcagtcatgtccaactctgtgcaaccccatggactgcagcctgccaggctcctctgtccatgggattctgcaggcaagaatactggagtgggttgccattccttgctccaggagatctttccgacccagggatcaaacccgcgtctcttatgtctcctgcattgggaggtgggttctttaacactagcactgcccccaccccaaggcacctactagctaggaaaggacaaggaagagtcagaaagtgacttccaaatctagcgatgttcacacttgaaagtcacataggctgagatctgcttaggtggcaagcaaacacagtattgcctgggtcacctacccaagctccttgcttctccaaggttcattaccctcctggcacatggtctctcccaacagagacgttgaacttggagacctggggtggagtccacaaaagctgcatattttgaagccatgaagcatcttttcacagatgttttctagatgtattagttttccattgccgcataacaaataacccccatgtttaaacccttcaaacaatgctcatttattagcttacatttctgggcattagagattgggtgggcttaagtggattctctgctctaggtctcgtgagactcaagtcaaggtgttggccagctggactcttatcactaggttctggcttaggagtcatttccaagttcatttgggctgtccctcaatttcagttttttgggttgtcaaatcagggcccccatttccttgctgtcaacagggagccactctcagctttcacaggatgcctacattccttgccagggacccacccctccagcttcaagtctgcaatgatgtgtccatttcttctcctggagaaagaaatggcaacccactccagtattcttgcctgcagaatcccttggacagaggagcctgacaggctacactccatggggtcacaaagaactggacatgactgaggcggctttggcatgcacatatgattagattaggcccatctagataatctttttgccacgtaatgtaattacagtagtaatgccagggggcaaaggtcatagggatcgccttaaaattctgcccaccccatgtaaagccaggctttctttcagtaaaagctaaacattaaacagtggtaccttgatggtgctgtatttgggagtaactacaggtaactgaaaatgcctgcaagttctgttccactttaacttccatctcgtaaggagctggcttttccttggctcttatgaatgggcaataattattgatcctcagatttctcaacatccatatgaaagttttttatattgtagtaagggtacagtagtgactgttgggagaaggcaatggcaccccactccagtactgttgcccggaaaattccatggatggaggagcctggtaggctgcagtccgtgtggtttcgaagagtcggacacgactgagcgacttcactttcactttccactttcatgcattggagaaggaaatggcaacctactccagtgttcttgcctggagaatcccatggatggacaagcctggtaggctgcagtccatggggtcacacagagtcggacacgactgaggcgacttagcagcagcagcagcagtgactgttagttcactaaccctagagcacgtacttggtataactcacactaaggagatgagttggattttagtgaaaaatacagtttagtaaagattggtctccacacccagtgaaatacgattttgagagttttttttttttttttttactataacccttgatcataccaatcacattaatcttttcattgctttgtataattacgaaaataatattttcagccccctgttaaggaagaatcgcagaaagtgagacactctactaaacattatagagtacgtacaaaaacattaaatgtcatgcccacaaaagtttacaaaagcaaaagaatggctattaataccagtgtttgtataagagatatataagtggtagcctccctacaaaaaactttttgagtgttgtctacatttgaggatatattctaacataaagatttcaaaatgagattgtgtagcatgatggtgaaaggctgttgaatcacgcgaatacaccaggattcttggcccccggaggagaagaattcaatccggggccagagacgaggcttgatcactcagagcttttgtgtaataaagttttattaaagtataaaggagatagagaaagcttctgacataggcataagaatggggcagaaagagtacccgcttgctagtgttaacaatgaagttatataatccaaagaatatctggaggttgtaaagacctcatcagacctactcccataatttacattttaagataacatggtcctcaggcaagatacatccttgtaaagatcaggtctactcccataattaacattttaagataacagaaggttgaatccaaagactgtccttaggcagaatacatgattgttatataatcctaaggaatgtggagaaagaaaaaaaatttgtcctttcttcctccttgagaattccagacccctctctccttggggacccctagactccctatcaacctgcctaggaaatggctctctcaatggttagtggttaaaagcaccatgttggaatctgatggactcgtctcacctctgcagtgtattatccttctgaaaattagttaagtctctgagttggagtctcacttacaacctatggataataatggaacctattgcattgtagggagcataaacatgggtgggaaaatgttcatgtttagcataacacgtgacactttgctattattataaaccaaaagtgtccactttcctgtcatgctgggacatgagagaatttgatacataccatcatagaggtatagacagattgtgggaaatatcaataaaatatatggttgtgtagataaggtagagagactctggcttggaaatcttgaaggatggcctagtagaagctggatctggactctccttggggctggccagcgtttccttgagttatgctaatagactgaagcaacagacatataccttgctctgctataggtctggcatgaaccatagccaggatccaacatcaggccccctgtgatggtggggcatcttgctgtgtgcttctgccaaagctttccatgttttcttgtcatcacatctcagataacttcatcttttgcttctttgaacatcaagaaactgaacagggtgaaaaactaaaaccattctgaacttactgcaatttctttaaccgttcagagaactcctacttcagaactcattcaaagtatggtcaaagcactgcgaatttcatcctgaaatgcctagaaaatactgccagtctaacattgtgactctactttgattttccttaggaaaacatgacccttcactttttgcttctgacatcccttttcctgctcatctctgattcctgtgagttcttcactgaagccagttatccccgaagctatccttgcgatgtgaaaaacgaaaatggctcttttattgcagaatgtaatggtcgtcgattacaggaagtaccccaaacagtggacaaagatgtgacggaagtagacctgtctgataatttcatcacacgggtaacgaatgaatcctttcaagggctgcaaaatctgactaaaatcaacctgaaccataatgccaagtcccagagtggaaatcctgctgtaaagaaagctatgactattacagacggggcatttctcaacctcaaacacctaagggagttgctgctggaagacaaccagttacaacaaataccagctggtttgccagaatctttaaaaaaacttagtctaattcaaaacaacataattacgttaacgaaaaagaacacttctggacttgggaacctggaaagtctctatttgggctggaactgttattttgcttgtgataaaaaatttaccatagaaaatggagcattccaaaaccttaccaagttgaaggtgctgtcattatcttttaatccccttcacagcgtgccaccaagtctgccaagctcgctaacagaactctaccttagtaatacccatattggaaacgtcagtgaagaagacttcaaggaactgagcaatttaagagtactagatttaagtggaaactgcccgagatgttttaacgctccatttccctgtgtaccttgccaaggagatgcttcaattcagatacaccctcttgcttttcaaaccctgacccaacttcgctacctaaacctctctagcacttcgctcaggaaggttcctgccagctggtttgacaacatgcacaatctgaaggtattggatcttgaattcaactatttaatggacgaaatagcctcgggggaatttttgacaaaattgccctccttagaaatacttgatttatcttacaactatgaactgaaaaaataccctcagtacattaacatttccaaaaatttctcgaagcttatatctctccagatgttgcatttaagaggttatgtgttccaggaacttagaaaggaagatttcgagcccctgcggaacctctcaaatttaacgactatcaacttgggcgttaactttattaagcagattgattttagtattttccactggttccccaacctgaaaatcatttacttgtcagaaaacagaatatcacccttggtcagtgataccgagcaacatgatgcaaatgggacctctttccaaagtcacatcctgaagcgacgctcagccgatattcaatttgacccacattcgaatttttatcataacacccgtcctttaataaagacagaatgttcacgtcttggcagtgccttagatttaagcttgaacagtattttctttattggggtaagccagtttaaagattttggcaacatttcctgtttaaatctatcttcaaatggcaatggccaggtgttaaatggaacggaattttcatgcttgtctggtatcaagtatttggatttgacaaacaatagactagactttgatgacgatgctgctttcagtgaattgccattgttagaagttctcgatctcagctacaatgcgcactatttccgaatagcaggggtaacgcaccgtctaggatttattgaacatttaactaacctgagagttttaaacttgagcaacaatgacatttttactttaacagaaacacaactgaaaagcgcgtccctgggagaattagttttcagtgggaaccgccttgaccttctgtggaatgctcaagatgtcaggtactggcaaatttttcaaaatctcaccaatctgacccggcttgacttagcccgtaataaccttcggcatatctccagtcaggccttccttaacttgcccaggactctcactgacctatatataaatgataacatgttaaatttctttaactggtcattactggaatacttccctcacctcagattgcttgacttaagtggaaaccagctgttctttttaaccaatagcctatctacatttgcatcttctcttgagacgctactgctgagtcgaaacagaatttcccacctgccgtctgattttctttctggagccagcagcctgatacacctcgatttgaactccaaccagctcaagatgctcaacagatccacatttgaaacgaagaccgccaccaagttaactgttttggaactagggggtaacccttttgactgtacctgtgacttcggagattttctagaatggatggacagaaatctgaacgtcagggttcccagactgaccgatgtcatttgtgccagtcctggggatcaagaaggcaagagcattgtgagtctagacctcagcacttgtgtttcagataccattgcagccatattctgtttcttaaccttttctgtcaccatctcagtgatgctggctgccctggcccaccactggttttactgggatgcttggtttatctaccatgtgtgcttagctaaggtcaaaggctacaggtctctgtccacatcccagactttctatgatgcttacatttcttatgacaccaaagacgcttctgtcacggactgggtgatcaatgaattgcgcttccacctggaagagagtgaggacaagaatgtgctcctgtgtttagaggaaagggattgggacccgggtctagccatcatcgacaacctcatgcagagcatcaaccaaagcaagaaaacaatatttgttttaaccaaaaaatatgccaaaaactggaattttaaaacggcattctacttggccttgcagaggctaatggaggagaatatggacgtgattgtctttattctgctggagccagtgttgcagcattcgcagtatttgagactgcggcagaggatctgcaagagttccatcctccagtggcctgataaccccaaggcggaaggcttgttttggcagagtctgaaaaatgtcgtcctaacagccaacgattcacggtataacaacttgtatgtcaattccattaagcaatactaactgatgttaagccaggggtcacaggcatagtcaaaatgcacagcaatgctccttttgttcttagctgtcacttgctgtataacagagtactccaaacttaatggcttaaaatgatacactttgttgtgtcatcgtctctgtggaccaggagtccgggcttgagttctggctcagggcccctcaaaggcggtaatctctctgtcagccagggccacaggcatctgctacggttagcatccacttcagagcctacatttgtgtggtggttttcaggactgagttctccctgggctgctggcccaaggctaccctcagttcgtggccacatggacggacccttgcccgtggcagctcgcttcagcaaagccagcaagagagacaggttgctgacacaatggaagtcgccatctttgtaatccaatcagggaagtgataacccctcactctggccatattctgattgttagaagtaaatcagcggccctgccagctccatgggagtgatcacctcagtcccgggaaagtagtctatgaccaaggcgggcagtcatggtaagctcgctcagcctgccatctcggttgtaaatggtgaacaccaggagcagggatcactatggccatcctgacagttggcctgctctgccttcttttcagtatctcaggacttttgtgactgtaagagttctgatgttaagttgctgtttagatttatcatatatccatggctatttggttatattatgttatggttgcattagtccttttataattactttttataaacactggctataatattttacttctaagatttagataccatttaaaggctaagatggatggtagttaagtttttttgtttgtttgtttgcttcttaaccattttttaagtgtgcagctaaattagaagcatttggagttatctgtcaatcaccattgctgtaaatcatgaagttaattattaaaatgcttcatttcacatcatgagcatataataaatacacagggaagtaaatctgtaataaaataaattacctgaatgtccattgacagatgaatggataaagaagaggcagtacatatatacaatggactcagctataagaaaaagaatgaaataatgccacttgcagcaacatggacggacctagaaattattgtactgagatatcacttatatgtggaatctgaaatatgacccaaagggatccatctgtggagcagaaccagactcattgacatcgagaacagacttgtgtttgccaaggagggtgggaggagatggactgggagtttcgggttagcagatgcaaactattacatctagaatagatggacaacaaagtcctactgtaaggcacagggaactatattcagtgtcctgggataaaccataatggaaaagaatatgaagaagagtgtatatatgtatataactgtcactttgctgtatacagaaattaaca

SEQ ID NO: 50 - DNA, 231 bp, Bos taurus
gcagccatgaaattaaaaggtacttgcttcttggaagaaaagctatgaccaacctagacagcatattaaaaagcagagacattactctgtcaacaaagttctgtctagtcaaagctatggtttttccagtggtcatgtatggatgtgagagttggactataaagaaagctgagtgccaaagaattgatgcttttgaactgcggtgttggagaagactcttgagagtccctt

SEQ ID NO: 51 - RNA, 20 nt, Artificial Sequence
  Description of Artificial Sequence Synthetic oligonucleotide
ucauguauggaugugagagu

SEQ ID NO: 52 - RNA, 20 nt, Artificial Sequence
  Description of Artificial Sequence Synthetic oligonucleotide
ucacauccauacaugaccac

SEQ ID NO: 53 - DNA, 154 bp, Bos taurus
actcgtctctgctcactggggcatcagctggagtgactcaactgggattggaagacccggtcccagtgtggcatactcagtggccatcttctggctgctccgttggagataccagcgagaccctcggttctcctccctgttgccttgcacaagg

SEQ ID NO: 54 - RNA, 20 nt, Artificial Sequence
  Description of Artificial Sequence Synthetic oligonucleotide
ugaguaugccacacugggac

SEQ ID NO: 55 - DNA, 161 bp, Bos taurus
caaagagtcagacacgactgagcgacttcactttcactttcactattatgaatttatcaatatattatcagtttgtgaaaagcaaagcaggaagtaatattgcgttttctctcttccttgtttatgaagtcattgatcacaggggcacaatttaaattgag

SEQ ID NO: 56 - RNA, 20 nt, Artificial Sequence
  Description of Artificial Sequence Synthetic oligonucleotide
uaugaagucauugaucacag

SEQ ID NO: 57 - DNA, 60 bp, Bos taurus
gtcccagtgtggcatactcagtggccatctactctcacatccatacatgaccactggaaa

SEQ ID NO: 58 - DNA, 60 bp, Bos taurus
actctcacatccatacatgaccactggaaagtcccagtgtggcatactcagtggccatct

SEQ ID NO: 59 - DNA, 60 bp, Bos taurus
ctgtgatcaatgacttcataaacaaggaaggtggtcatgtatggatgtgagagttggact

SEQ ID NO: 60 - DNA, 60 bp, Bos taurus
gtggtcatgtatggatgtgagagttggactctgtgatcaatgacttcataaacaaggaag